(19) United States
(12) Patent Application Publication
(10) Pub. No.: US 2012/0009158 A1
(43) Pub. Date: Jan. 12, 2012

(54) VENTRICULAR INDUCED PLURIPOTENT STEM (ViPS) CELLS FOR GENERATION OF AUTOLOGOUS VENTRICULAR CARDIOMYOCYTES AND USES THEREOF

(75) Inventors: Kenneth R. Chien, Cambridge, MA (US); Huansheng Xu, Malden, MA (US); Byungdoo Alexander Yi, Boston, MA (US)

(73) Assignee: THE GENERAL HOSPITAL CORPORATION, Boston, MA (US)

(21) Appl. No.: 13/163,339

(22) Filed: Jun. 17, 2011

Related U.S. Application Data
(60) Provisional application No. 61/356,447, filed on Jun. 18, 2010.

(51) Int. Cl. A61K 35/12; C12Q 1/02; A61P 9/00; C12N 5/071; C12N 15/85

(52) U.S. Cl. 424/93.7; 435/377; 435/455; 435/325; 435/366; 435/29

(57) ABSTRACT

The present invention generally relates to methods and compositions to generate a secondary iPS (2iPS) cell to produce somatic cells of a rare differentiation cell type fate. In some embodiments, the method relates to an increase in efficiency of differentiation and production of high yields of somatic cells of a rare differentiation cell type fate produced from secondary iPS (2iPS) cells as compared to their differentiation from other pluripotent stem cell sources such as ES cells or primary iPS cells. In some embodiments, the present invention relates to compositions, methods and systems for reprogramming a first somatic cell into a primary iPS cell, where the primary iPS cell is then differentiated along a selected linage to produce a second somatic cell, which is then reprogrammed to a secondary iPS cell (2iPS) cell. The 2iPS cell has a high efficiency of differentiating into a cell of the same cell type as the second somatic cell, e.g., a somatic cells of a rare differentiation cell type fate such as but not limited to a ventricular cardiomyocyte, a pancreatic β-cell or a hepatic cell. In some embodiments, the first somatic cell is a fibroblast, or a cardiac cell, but is not limited to cardiac fibroblast cells. In some embodiments, the present invention relates to compositions, methods and systems to produce ventricular cardiomyocytes from secondary induced pluripotent stem cells (iPSC), where the iPSC are themselves generated from ventricular cardiomyocytes. The secondary iPS (2iPS) cell generated from ventricular cardiomyocytes have a higher cardiomyogenic potential and high cardiomyogenic yield as compared to primary iPSC, and are useful in drug discovery, disease modeling and cell-based therapy.

### CROSS REFERENCE TO RELATED APPLICATIONS

This application claims benefit under 35 U.S.C. 119(e) of U.S. Provisional Patent Application Ser. No. 61/356,447 filed on Jun. 18, 2010, the contents of which is incorporated herein in its entity by reference.

### GOVERNMENT SUPPORT

This invention was made with Government support under Grant Number 5T32HL007208-32 awarded by the National Institutes of Health (NIH). The U.S. Government has certain rights in this invention.

### FIELD OF THE INVENTION

The present invention relates to the field of tissue, organ and cell transplantation. Methods and compositions relate to the use of an inducible secondary iPS (2iPS) cell system to produce ventricular cardiomyocytes from induced pluripotent stem cells (iPSC), where the iPSC are themselves generated from ventricular cardiomyocytes. The secondary iPS (2iPS) cell generated from ventricular cardiomyocytes have a higher cardiomyogenic potential and high cardiomyogenic yield as compared to primary iPSC, and are useful in drug discovery, disease modeling and cell-based therapy.

### BACKGROUND OF THE INVENTION

Cardiovascular disease is the leading cause of death in the U.S., and will be the primary cause of mortality in developing countries by 2010, as estimated by the WHO. Nevertheless, the demand for transplantation exceeds the availability of donor hearts. In this regard, cardiac regeneration has recently become an active area of research. Over the past few years, numerous reports demonstrate cardiac progenitors from diverse fetal and adult tissues outside the cardiovascular system, including adipose tissues, amniotic fluid, bone marrow, placenta, skeletal muscle, and testes. However, their low frequency of cardiac differentiation (Murry et al., 2004) and lack of long-term benefits fail to achieve cardiac cell regeneration (Fazel et al., 2006, 2008). Bone marrow cells, for instance, may improve the function of the infarcted heart mainly by promoting angiogenesis or cell survival without cardiac muscle regeneration (Fazel et al., 2006, 2007). A recently discovered cardiac progenitor population marked by the expression of the LIM homeodomain transcription factor isl1 (Laugwitz et al., 2005; Moretti et al., 2006) is an attractive target to study cardiac regeneration. A multipotent islet 1 (isl1+) cardiovascular progenitor (MICP) is able to give rise to the major three cell types of the heart: cardiomyocytes, smooth muscles and endothelial cells, and has clonogenic and self-renewing ability (Laugwitz et al., 2005; Moretti et al., 2006). In Isl1 knockout mice, histological analysis of mutant hearts between embryonic day (ED) 9.0 and ED9.5 showed a misshapen single heart ventricle as the cause of death (Cai et al., 2003). Lineage tracing studies in mice document that isl1+ progenitors give rise to over two thirds of the cells in the heart, mostly on the right side, including most of the conduction system: the sinoatrial (SA) node, the atrioventricular (AV) node, His-bundle, and Purkinje fiber complex (Cai et al, 2003; Laugwitz et al., 2005; Moretti et al., 2006; Sun et al., 2007). Disruption of development, differentiation or maturation of any of these components can lead to arrhythmias such as sinus arrest, AV block, ventricular tachycardia and sudden death (Bruneau et al., 2001).

ES cells hold great promise for regenerative medicine with their capability to expand indefinitely and to differentiate into all the cell types found in an adult body, including cardiomycytes. However, the efficiency of ES cells to differentiate into cardiomyocytes is usually very low and the purification of the resultant cardiomyocytes remains a challenging task to fulfill the regenerative potential of ES cells for heart diseases (Laflamme, Chen et al, 2007). To address the problem, one approach is to optimize the differentiation protocols to increase the cardiomyogenic efficiency, while the other one is to start with cell lines of a higher cardiomyogenic potential.

Recently, it has been demonstrated that differentiated somatic cells can be reprogrammed to a pluripotent state by forced expression of several transcription factors (Takahashi and Yamanaka 2006; Maherali 2007; Wernig, Meissner et al. 2007). The expression of the factors can cause genome-wide epigenetic remodeling, reactivate the expression of stem cell genes and turn differentiated somatic cells to induced pluripotent stem (iPS) cells. The resultant iPS cells resemble ES cell in many aspects, including the capacity of self-renewal and the pluripotency. iPS cells also provide the unprecedented opportunity to generate autologous somatic cells in large quantity. However, the cardiomyogenic potential of iPS cells may be even lower than that of ES cells (Mauritz, Shwanke et al, 2008; Zhang, Wilson et al, 2009), which limited their application in regenerative cardiology.

The generation and expansion of diverse cardiovascular cell lineages is a critical step during human cardiogenesis with major implications for congenital heart disease. Unraveling the mechanisms for human heart cell lineage diversification has been hampered by the lack of genetic tools to purify early cardiac progenitors and define their developmental potential.

### SUMMARY OF THE INVENTION

The present invention relates generally to compositions and methods of use and production of Ventricular induced pluripotent stem (ViPS) cells. ViPS are iPS cells from ventricular cardiomyocytes. ViPS cells are similar to ES cells and iPS cells from skin fibroblasts in many ways, including the self-renewal capability, but they distinguish from those cells with a much higher cardiomyogenic potential. When differentiated, ViPS cells start to generate beating cardiomyocytes at least two days earlier than ES cells or skin-iPS cells, and the yield of cardiomyocytes from ViPS cells can be 10-fold greater as compared to yield of cardiomyocytes from ES cells or skin-iPS cells. ViPS cells differentiate into cardiomyocytes and most ViPS cell-derived cardiomyoctes are of ventricular type, demonstrating that ViPS cells retain some property of their parental ventricular cardiomyocytes, which makes them more cardiomyogenic. This concept may be extended to other tissues. By employing the same technology, the inventor can generate iPS cells from other tissues and use them to produce large numbers of desired cell types that are usually rare out of differentiation of ES cells, i.e., skeletal myoblasts or insulin-producing pancreatic β-cells.

A central goal of cardiac regenerative medicine is to be able to generate sufficient numbers of ventricular myocytes necessary for effective cardiac cell replacement therapy. Here, the inventors demonstrate that induced pluripotent stem cells derived from murine ventricular cardiomyocytes (ViPS cells) can give rise to markedly higher numbers of relatively pure ventricular myocytes than genetically matched iPS cells derived from fibroblasts or embryonic stem (ES) cells and this higher efficiency of cardiomyogenesis occurs through an Islet1⁺ cardiac progenitor intermediate. The ventriculogenic bias of ViPS cells is due in part to epigenetic signatures retained from the starting ventricular myocyte. Herein, the inventors demonstrate how stem cell memory can be exploited to derive abundant numbers of ventricular myocytes from pluripotent stem cells and addresses one of the central challenges in cardiac regenerative medicine.

It has been reported that the iPS cells generated from different parental cell types can behave differently (Aoi, Yae et al. 2008), which led the inventors to assess whether iPS cells with different origins may retain some properties of their parental cells. Is this the case, the inventors demonstrate herein that iPS cells from cardiomyocytes may have a higher-than-ES-cell cardiomyogenic potential. In contrast, the iPS cells from a non-cardiomyocyte origin may not only have a lower-than-ES-cell cardiomyogenic potential, they may also generate defective cardiomyocytes. In particular, the inventors demonstrate that iPS cells from ventricular cardiomyocytes may be optimal to generate cardiomyocytes of a ventricular identity. Therefore, the inventors started to reprogram cardiomyocytes to iPS cells and focused on ventricular cardiomyocytes.

The inventors have successfully generated iPS cells from mouse ventricular myocytes (2iPS-VM cells, herein also known as ViPS cells) and genetically matched secondary iPS cells from tail tip fibroblasts (herein referred to as 2iPS-TTF or TiPS cells) through an inducible secondary iPS (2iPS) cells system. The inventors evaluated their cardiomyogenic potential and the data suggest that ViPS cells have a higher cardiomyogenic potential than TiPS cells, and the cardiomyocytes from ViPS are mostly ventricular myocytes instead of atrial myocytes. ViPS cells are also better than ES cells in terms of cardiomyogenesis.

Accordingly, the inventors have demonstrated that they can produce a high yield of cardiomyocytes from ViPS cells. ViPS cells are iPS cells which are reprogrammed from cells of cardiac lineage, e.g., cardiomyocytes, and in particular ventricular cardiomyocytes. In some instances, the cells of cardiac linage, (e.g., cardiomyocytes, and in particular ventricular cardiomyocytes) are themselves differentiated from primary iPSCs. In such instances, such reprogramming of the primary iPS cell derived-cells of cardiac linage, (e.g., cardiomyocytes, and in particular ventricular cardiomyocytes) can produce secondary iPS cells, e.g., 2iPS cells.

Stated another way, ViPS cells can be derived from ventricular cardiomyocytes that themselves are differentiated from (primary) iPS cells. The primary iPS cells can be generated from the reprogramming of any somatic cell, including fibroblasts, cardiac fibroblasts, and cardiac somatic cells such as cardiomyocytes. The ViPS cells and methods of use and compositions as disclosed herein are distinct from other secondary iPS cells, for example, as disclosed in US application US2010/0062534 in that in the present application, the secondary iPS cells are generated from the reprogramming of cells of cardiogenic lineage, and in particular from ventricular cardiomyocytes, whereas the US2010/0062534 application only discloses generation of secondary iPS cells from fibroblasts. Additionally, unlike other secondary iPS cell populations, in some embodiments, the secondary iPS cell population as disclosed herein relates to a secondary iPS population generated from the reprogramming of adult or neonatal cardiac cells, rather than a secondary iPS population of cells generated from the reprogramming of embryonic cardiac cells.

Accordingly, as disclosed herein, a colony of 1iPS cells (e.g., primary iPS cells) can be generated with a inducible expression vector expressing reprogramming factors, where the 1iPS cells (e.g., primary iPS cells) are selected and differentiated in vitro to yield a cell of cardiogenic lineage, e.g., such cells can be ventricular cardiomyocytes or other cardiac cells. These cardiogenic cells harbor the inducible viral transgenes required for reprogramming a second time (to produce “secondary” pluripotent cells or “2iPS” cell). Accordingly, the cardiogenic cells maintain the same viral integrations that mediated the initial conversion to 1iPS cells, however the proportion of cells carrying the necessary re-programming factors is increased. These cardiogenic cells can be readily induced to form 1iPS cells without further need for direct viral infection. This “secondary iPS cell” system produces a population of cardiogenic cells that can inducibly and homogeneously express the reprogramming factors with improved efficiency. In addition, the use of secondary pluripotent cells (e.g., 2iPSCs) can speed the production of 1iPS cells, providing for a faster, more efficient system for 1iPS cell induction. Such a system provides a powerful tool for mechanistic analysis, chemical and genetic screening for factors that enhance or block reprogramming, and the optimization of 1iPS cell derivation methods. In addition, the 2iPS cell composition provides the added advantage of being cultured as a cardiogenic cells phenotype until an iPS cell phenotype is desired.

In alternative embodiments, the primary iPS cell population can be generated by methods other than introduction of an inducible viral transgene construct, and can be created using synthetic modified RNA, as disclosed herein. Accordingly, such primary iPSC can be subsequently differentiated along cardiogenic lineages and then reprogrammed a second time using similar synthetic modified RNAs to produce secondary iPSCs (2iPSCs), which have a higher efficiency of differentiation along cardiac lineages, such as for example ventricular cardiomyocytes.

The human counterpart of the above disclosed mouse ViPS cells will enable one to easily obtain a high yield and/or high-quality, in some instances, patient-specific (e.g., autologous) ventricular cardiomyocytes in high-yields and at high efficiency. In some embodiments, autologous ventricular cardiomyocytes and/or cardiac cells will be useful to study the mechanism of various heart diseases and to test the efficacy and cardiotoxicity of drugs. Additionally, cardiac cells differentiated from 2iPS cells, e.g., ventricular cardiomyocytes are also useful in method for the treatment of heart failure, which is one of the leading causes of death and costs billions of dollars each year in America. The concept that the iPS cells of a certain origin may preferentially differentiate back to their parental cell type may be generalized to other tissues, and will help to identify optimal iPS cells for the production of corresponding somatic cells with high yields and quality.

By using a doxycycline inducible system, mouse ventricular induced pluripotent stem cells (hereafter referred to as ViPS cells) with higher cardiomyogenic potential have been generated from ventricular cardiomyocytes. These cells can be used to generate high-quality autologous ventricular cardiomyocytes with high yield.

Another aspect of the present invention relate to the use of human ViPS cells to generate high-quality autologous ventricular cardiomyocytes with high yield to test the efficacy and toxicity of drugs.

One aspect of the present invention relates to a method of generating an induced pluripotent stem (iPS) cell with a high efficiency to differentiate into ventricular cardiomyocytes, comprising (a) reprogramming a first somatic cell, e.g., cardiac fibroblast cell, by inducing the expression of at least one reprogramming factor to produce a first pluripotent stem (iPS) cell (also called a primary iPS cell herein); (b) directing the differentiation of the first induced pluripotent stem (iPS) cell along the lineage of a second cell type to generate a second somatic cell, e.g., differentiating along a cardiomyogenic lineage to produce a second somatic cell which is a ventricular cardiomyocyte; and (c) reprogramming the second somatic cell, e.g., ventricular cardiomyocyte, by contacting it with at least one reprogramming factor to produce a second induced pluripotent stem (2iPS) cell, e.g., a ViPS cell which has a high efficiency of differentiating to ventricular cardiomyocytes.

In some embodiments, a first somatic cell can be any cell type that can be reprogrammed into a primary iPS cell, which is then differentiated along the same or a different lineage of the first somatic cell type to produce a second somatic cell, which is subsequently reprogrammed to a secondary iPS (2iPS) cell, where the 2iPS cell has high efficiency of differentiating along the lineage to produce a cell that is the same cell type as the second somatic cell. In some embodiments, a first somatic cell is from a postnatal or adult subject. In some embodiments, a first somatic cell is not an embryonic cell, e.g. an embryonic fibroblast. In some embodiments, the first somatic cell can be any cell type that can be reprogrammed into a primary iPS cell, which is then differentiated along a different lineage of the first somatic cell type to produce a second somatic cell which is of a different cell type of the first somatic cell, e.g., a first somatic cell can be a fibroblast, including skin or cardiac fibroblast, and a second somatic cell can be a ventricular cardiomyocyte. In an alternative embodiment, the first somatic cell can be any cell type that can be reprogrammed into a primary iPS cell, which is then differentiated along the same lineage of the first somatic cell type to produce a second somatic cell which is of a same cell type of the first somatic cell, e.g., a first somatic cell can be a ventricular cardiomyocyte, and a second somatic cell can be a ventricular cardiomyocyte.

In some embodiments, the first somatic cell is a cardiac fibroblast cell. In some embodiments, a first somatic cell is a cardiomyocyte, e.g., a ventricular cardiomyocyte.

In some embodiments, a first somatic cell can be any cell type, such as a pancreatic cell, a liver cell or a lung cell. In some embodiments, the first somatic cell can be a cell which is a rare differentiated cell type, e.g., a pancreatic β-cell or neuronal cell or the like.

In some embodiments, where the first somatic cell is from a particular tissue or cell type, then the primary iPS cell derived from the reprogramming of the first somatic cell is differentiated to a lineage of the same or different tissue or cell type to generate a second somatic cell. The second somatic cell can be reprogrammed to a second iPS (2iPS) cell and then differentiated to a cell of the same cell type of the second somatic cell. The difference between the first (or primary) iPS cell and second iPS (2iPS) cell is that second iPS (2iPS) cells have a much higher efficiency to differentiate to a cell of the second somatic cell type as compared to cells differentiated from primary iPS cell. In some instances, the second somatic cell type can be very difficult to produce from the differentiation from ES cells or the first iPS cells. Accordingly, the inventors have surprisingly discovered a method to increase the efficiency and yield of a cell of a rare differentiated cell type fate (e.g., such as a ventricular cardiomyocyte or pancreatic islet β-cell) by differentiating the cell from a secondary iPS cell rather than a primary iPS cell.

The advantage of using a secondary iPS (2iPS) cell system as disclosed herein is that it results in an increased efficiency and increased yield of producing somatic cells of a rare differentiated cell type which are typically produced at low efficiency and low yield when differentiated from other pluripotent sources such as ES cells or the primary iPS cells. For example, for certain somatic cell types, e.g., ventricular cardiomyocytes, are very difficult, if not impossible, to be reprogrammed to iPS cells through direct reprogramming, e.g., through increasing the expression of reprogramming factors via transfection with transcripts or viruses expressing the reprogramming factors. By using the secondary iPS cell system as disclosed herein, the first iPS cell can be generated from any cell type that is easily reprogrammed to a primary iPS cell by any method known by ordinary skill in the art and as disclosed herein, e.g., with lentivirus or protein reprogramming factors or small molecules which replace the reprogramming factors and the like, where the primary iPS cell is then differentiated to a second somatic cell of interest. In some embodiments, if the reprogramming of the first somatic cell to a primary iPS uses an inducible-expression construct which encodes the reprogramming factors in first somatic cell where the transcripts encoding the reprogramming factors are integrated into the first somatic cell genome, the second somatic cell, will have incorporated the reprogramming transgenes, i.e., Klf4, Oct4, Sox2 and c-Myc, into its genome, and therefore, there is no need for a second viral infection of the second somatic cell, and the expression of the reprogramming transgenes can be induced by contacting the second somatic cell with the inducible agent, e.g., a doxycycline. For example, where the primary iPS is generated by contacting a first somatic cell with a lentivirus expressing the reprogramming factors under the control of an inducible promoter, reprogramming the second somatic cell will require contacting the second somatic cell with an agent which induces expression from the inducible promoter, e.g., doxycycline to reprogram the second somatic cell to the second iPS (2iPS) cell. Any inducible promoter known to one of ordinary skill in the art can be used control the expression of at least one or more reprogramming factors to reprogram the first somatic cell into a primary somatic cell, and any coordinated agent which turns on the expression of the genes controlled by the inducible promoter can be used to reprogram the second somatic cell to a secondary iPS (2iPS) cell.

For example, in some embodiments, the somatic first cell, e.g., a cardiac cell, e.g., a cardiac fibroblast cell can be reprogrammed into a primary iPS cell. In some embodiments, for reprogramming to a primary iPS cell, a population of cardiac fibroblast cells are infected with lentiviruses and the genes encoding the reprogramming factors (Klf4, Oct4, Sox2 and c-Myc) are under the control of the doxycycline-inducible promoter are introduced into the genome of the cells and remain there thereafter. Doxycycline is added to the medium to induce the expression of the transgenes encoding the reprogramming factors during the reprogramming. After the primary iPS cells are obtained, doxycycline is withdrawn from culture and the transgenes are silenced. The primary iPS cells are then differentiated to various somatic cell types, including the desired second somatic cell. In some embodiments, the second somatic cell is a cardiac cell, e.g., a ventricular cardiomyocyte, which is difficult to be reprogrammed by infecting with a lentivirus expressing the reprogramming factors. In some embodiments, the second somatic cell, e.g., a cardiac cell, e.g., a ventricular cardiomyocyte, which because it was derived from the primary iPS cell still carries the reprogramming transgene in its genome, but does not express them without the inducing drug, doxycycline, is reprogrammed to a secondary iPS (2iPS) cell by simply culturing at the presence of the agent which induces expression from the inducible promoter, e.g., where the agent doxycycline. In some embodiments, such a secondary iPS (2iPS) cell can spontaneously differentiate into ventricular cardiomyocytes at a high efficiency and results in a high yield of ventricular cardiomyocytes.

In alternative embodiments however, where integration of genes encoding reprogramming factors into the genome of a somatic cell is not desirable, e.g., for clinical or therapeutic use of cells of a rare differentiation cell type fate which are differentiated from a 2iPS cell, one can reprogram the first somatic cell and the second somatic cell with non-viral reprogramming methods e.g., proteins or RNA transcripts which replace the reprogramming factors, small molecules or RNAi agents which increase the expression of the reprogramming factors in the first or second somatic cell being reprogrammed. Any means known to one of ordinary skill in the art to reprogram a somatic cell is contemplated for use in any and all aspects of the methods and compositions of the present invention, such as the methods or variations of such methods disclosed herein and in the patents and patent applications disclosed herein. In some embodiments, the expression of the reprogramming factors in the first somatic cell and/or the second somatic cell is by a non-integrating viral vector as disclosed herein. In some embodiments, the expression of the reprogramming factors in the first somatic cell and/or the second somatic cell is by a non-viral polycystronic vector. In some embodiments, a primary or secondary iPS cell has minimal viral remnants if the secondary iPS or differentiated cells derived therefrom is administered to a subject for therapeutic use.

In some embodiments, the first and/or second cardiac cell is a cardiac fibroblast, such as a human cardiac fibroblast. In some embodiments, the first and second cardiac cell is a cardiomyocyte, e.g., a ventricular cardiomyocyte or a human cardiomyocyte. In some embodiments, a first or second cell is a pancreatic cell or a liver cell.

In some embodiments, contacting the first induced pluripotent stem cell with a reprogramming factor comprises contacting the first induced pluripotent stem cell with an agent which induces the expression of at least one reprogramming factor operatively linked to an inducible promoter, fore example, where the reprogramming factor is selected from one or more from the group consisting of: Oct family gene, Klf family gene, Sox family gene, Myc family gene, e.g., one or more selected from the group consisting of: Oct3/4, Klf4, Sox2, c-Myc, Nanog, Lin28.

In some embodiments, differentiating the first induced pluripotent stem cell along cardiogenic lineages comprising contacting the first induced pluripotent stem cell with a cardiomyocyte differentiating agent selected from the group consisting of: cardiotrophic agents, creatine, carnitine, taurine, activin A, activin B, insulin-like growth factors, bone morphogenic proteins, fibroblast growth factors, platelet-derived growth factor natriuretic factors, insulin, leukemia inhibitory factor (LIF), epidermal growth factor (EGF) and TGFα. In some embodiments, a second cardiac cell expresses at least one of any of the following cardiac markers; Mlc2v, cTnT.

In some embodiments, where the second iPS cell is derived from a second somatic cell which a cardiac cell, the second iPS cell expresses at least one of any of the following markers at a significantly higher level than ES cells or a primary iPS cell; Nkx2.5, Gata4, Isl1, myocardin and Tbx20.

Another aspect of the present invention relates to a ventricular cardiomyocyte obtained by reprogramming a secondary induced pluripotent stem (2iPS) cell, where the 2iPS cell was derived from reprogramming a second cardiac somatic cell, where the second somatic cardiac cell has differentiated from a primary iPS cell obtained from reprogramming a first cardiac cell.

In some embodiments, the ventricular cardiomyocyte is a mammalian ventricular cardiomyocyte, e.g., a human ventricular cardiomyocyte.

One aspect of the present invention relates to a method of generating an induced pluripotent stem (iPS) cell with a high efficiency to differentiate into a ventricular cardiomyocyte comprising: (i) reprogramming a first cardiac cell by contacting it with at least one reprogramming factor to produce a first induced pluripotent stem cell; (ii) differentiating the first induced pluripotent stem cell along cardiogenic lineages to generate a second cardiac cell; (iii) reprogramming the first cardiac cell by contacting it with at least one reprogramming factor to produce a second induced pluripotent stem cell.

In another embodiment, a first somatic cell is any cell which can be easily reprogrammed, such as e.g., a skin fibroblast cell or can be a pancreatic cell e.g., a cell type from the endocrine pancreas or exocrine pancreas, and can be reprogrammed into a primary iPS cell which is differentiated along pancreatic lineages to a second somatic cell. Methods to direct the differentiation of iPS cells along pancreatic lineages are well known by persons of ordinary skill in the art. In some embodiments, the second somatic cell is a pancreatic cell, e.g., a Pdx+ pancreatic cell or an exocrine cell or a pancreatic β cell. In some embodiments, the second somatic cell, e.g., a Pdx+ pancreatic cell or an exocrine cell or a pancreatic β cell is reprogrammed to a secondary iPS (2iPS) cell. In some embodiments, such a secondary iPS (2iPS) cell can spontaneously differentiate into Pdx+ pancreatic cell or an exocrine cell or a pancreatic β cell at a higher efficiency and higher yield as compared to from a cell differentiated directly from a primary iPS cell or ES cell.

In another embodiment, a first somatic cell, such as skin fibroblast, can be reprogrammed into a primary iPS cell with the inducible reprogramming transgenes. The primary iPS cells can be differentiated along liver lineages to a second somatic cell. Methods to direct the differentiation of iPS cells along liver and hepatic lineages are well know by persons of ordinary skill in the art. In some embodiments, the second somatic cell is a liver cell, e.g., a hepatic cell. In some embodiments, the second somatic cell, e.g., liver cell, e.g., a hepatic cell is reprogrammed to a secondary iPS (2iPS) cell. In some embodiments, such a secondary iPS (2iPS) cell can spontaneously differentiate into liver cells, e.g., a hepatocyte at a higher efficiency and higher yield as compared to from a cell differentiated directly from a primary iPS cell or ES cell.

Another aspect of the present invention relates to a method for stem cell therapy comprising; (a) collecting at least a first cardiac cell from a subject; (b) reprogramming the first cardiac cell from the subject into a first induced pluripotent (iPS) cell; (c) differentiating the first induced pluripotent (iPS) cell into a second cardiac cell e.g., a ventricular cardiomyocyte; (d) reprogramming the second cardiac cell, e.g., a ventricular cardiomyocyte into a second induced pluripotent (2iPS) cell; and (e) differentiating the second iPS cell into a cell of same cell type of the second cardiac cell, e.g., a ventricular cardiomycoyte and transplanting the differentiated cell from the 2iPS cell, e.g., a ventricular cardiomyocyte into the subject.

Another aspect of the present invention relates to the use of the method for stem cell therapy as disclosed herein to treat a subject with a cardiovascular disease or disorder.

Another aspect of the present invention relates to the use of a population of ventricular cardiomyocytes differentiated from a ViPS cell in an assay to evaluate the toxicity of an agent, wherein a population of ventricular cardiomyocytes are contacted with an agent compound and the contractile activity of a population of ventricular cardiomyocytes is measured. In some embodiments, the contractile activity of a population of ventricular cardiomyocytes in the presence of the agent is compared to the contractile activity of a population of ventricular cardiomyocytes in the absence of the agent. In some embodiments, a change in the contractile activity by a statistically significant amount in the presence of the agent as compared to the contractile activity in the absence of the agent identifies an agent that alters the contractile activity.

In some embodiments, a change in the contractile activity is an increase or decrease in at least one contractile activity parameter, and wherein a contractile activity parameter is selected from the group consisting of: contractile force, contractile frequency, contractile duration and contractile stamina.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1B shows a schematic of the doxycycline inducible system used to generate the secondary iPS cells. FIG. 1A is a schematic of the inducible promoter used to induce the expression of the reprogramming transgenes Oct3/4, Klf4, Sox2 and c-Myc in the presence of Dox (deoxyclycine). FIG. 1B shows that the presence of Dox increases the number of Ds-Red+ cells after infection of a lentivirus carrying an inducible dsRed transgene. Over 90% of the cells express Ds-Red+ in the presence of Dox.

FIG. 2 is a schematic showing one embodiment for the generation of 2iPS-VM (ViPS) (secondary iPS cells from ventricular cardiomyocytes) and 2iPS-TTF (TiPS) (secondary iPS cells from tail tip fibroblasts) through an inducible secondary iPS (2iPS) cell system. The strategy to generate iPS cells from murine ventricular cardiomyocytes and tail tip fibroblasts using an inducible secondary iPS cell system. In this embodiment, cardiac fibroblasts were collected from the compound transgenic (Mlc2v^{Cre/+}; Rosa26^{YFP/rtTA}; TgMef2c-AHF-dsRed) neonatal mouse pups. Mlc2v^{Cre} is a knock-in allele. Together with Cre-dependent Rosa26YFP conditional reporter allele, it can specifically label ventricular cardiomycoytes. Rosa26^{rtTA} is a knock-in allele that constitutively expresses rtTA, the transactivator of the doxycycline-inducible promoter. TgMef2c-AHF-dsRed is a transgene that labels the cardiac progenitors from the anterior heart field (AHF) and their progeny. Cardiac fibroblasts [Mlc2v^{Cre/+}; Rosa26^{floxYFP/rtTA}; TgMef2c-AHF-dsRed] were isolated and transduced with separate lentiviruses containing Dox-inducible reprogramming factors, Oct4, Sox2, Klf4, and Myc, in order to derive iPS cell clones. The infected cells were cultured with doxycycline for two weeks until iPS cell (“primary iPS cells”) colonies emerged. Primary iPS cells were injected into blastocysts to generate chimeras. Cardiomycytes in the ventricules were collected from one neonatal chimera and cultured with doxycycline. Some of the ventricular cardiomyocytes [referred to as “ventricular myocytes (VM)”] derived from the primary iPS cells were labeled by Ml2c^{Cre}-Rosa26^{YFP} and carried the genes expressing the reprogramming factors were reprogrammed to the secondary iPS cells (“ViPS”). In parallel, tail tip fibroblasts [“tail tip fibroblasts (TTF)”] were collected from the same chimera and reprogrammed to the secondary iPS cells (“TiPS”). One advantage of this is that it enables direct comparison of secondary iPS cell lines from different sources, e.g., ViPS or TiPS cells which have the same level of exposure to the reprogramming factors, as each secondary iPS cell line has the same inducible genetic construct for inducible expression of Oct3/4, Klf4, Sox2, and c-Myc, thereby reducing any adverse consequences due to location or copy numbers of the transgenes. Tail tip fibroblasts (TTF) and ventricular myocytes (VM) were isolated from a single postnatal day 0 (P0) chimeric pup. After isolation, the cells were seeded onto an irradiated mouse embryonic fibroblast feeder cell layer and Dox was applied for two weeks. Secondary iPS cells were picked on the basis of ES cell-like colony morphology and Dox-independent growth. Almost all the ViPS cells express YFP demonstrating an origin of ventricular cardiomyocytes, whereas the TiPS are negative for YFP. FIG. 2 thus shows schematic flow diagram outlining the steps for generating secondary ViPS (ventricular iPS) cells. Tail tip and cardiac fibroblasts are contacted with three reprogramming factors, Klf4, Oct3/4 and Sox2, and in some embodiments, c-Myc, to reprogram to produce primary iPS cells. The primary iPS cells are injected into a blastocyst of a non-transgenic mouse to generate a chimeric mouse, then tail tip fibrobasts (TTF) or cardiac fibroblasts (CF) were obtained from the chimeric mouse and cultured in the presence of doxycycline (Dox) to induce the expression of the reprogramming factors Klf4, Oct3/4 and Sox2, and c-myc in the chimeric-TTF or chimeric-CF to generate secondary iPS-TTF and secondary iPS-CF cells, respectively. The secondary iPS-CF have a higher efficiency to differentiate along cardiomyogenic lineages as compared to cells differenated from secondary iPS-TTF.

FIG. 3A-3D shows the characterization of ViPS (also referred to as ViPS) and TiPS cells. FIG. 3A shows whole mount fluorescence microscopy of a P3 heart (left panel, bright field) from Mlc2v^{Cre/+}; Rosa26^{floxYFP/rtTA}; TgMef2c-AHF-dsRed compound transgenic mouse. Both ventricles (RV and LV) are labelled by YFP (middle panel) whereas the right ventricle (RV), pulmonary trunk (PT) and ascending aorta (Ao) are labelled by dsRed (right panel). RA and LA: right atrium and left atrium. Scale bar, 500 μm. FIG. 3B shows whole mount fluorescence microscopy of a P0 heart (left panel, bright field) from chimeric animal that was derived by blastocyst injection of primary iPS cells. After factor-based reprogramming and blastocyst injection, the double reporter system remains active with iPS cells that had contributed to both ventricles (RV and LV) being labelled with YFP (middle panel) and iPS cells that had contributed to the right ventricle (RV), pulmonary trunk (PT) and ascending aorta (Ao) labelled by dsRed (right panel). RA and LA: right atrium and left atrium. Scale bar, 500 μm. FIG. 3C shows bright field image of tail tip fibroblasts (left panel) isolated from the chimeric pup and ventricular myocytes (middle panel) that are also YFP-positive (right panel). Scale bar, 20 μm. FIG. 3D shows ES cell-like colonies that emerged upon seeding TTFs and VMs onto irradiated mouse embryonic fibroblasts (MEF) in ESC medium with doxycycline. Colonies derived from VMs retain expression of YFP. Scale bar, 200 μm. FIG. 3D also compares the morphology and alkaline phosphatase (AP) activity of ViPS and TiPS. Both ViPS and TiPS express AP, and only ViPS express YFP, whereas TiPS do not express YFP.

FIGS. 4A-4H ViPS cells display increased cardiomyogenic potential in vivo and in vitro. The embryos were collected at E13.5. Hearts of the embryos are shown in the insets. To visualize TiPS cells, the conditional reporter allele Rosa26^{YFP} was activated by a transient expression of an exogenous Cre. Images of E13.5 embryos obtained by injecting ES cells, ViPS cells or TiPS cells into 2n blastocysts. The pattern of ES/iPS cell contribution to the chimeras could be classified into three types: FIG. 4A shows type I chimeras demonstrated an overall ≧50% contribution to the entire chimera and a similar contribution to the heart; FIG. 4B shows type 2 chimeras demonstrated a <50% contribution overall but a much higher contribution to the heart; and FIG. 4C shows type 3 embryos that demonstrated a <50% contribution overall with an equal or lesser contribution to the heart. Scale bar, 2 mm. FIG. 4D shows a graph of the patterns of contribution of ViPS cells, TiPS cells, and ES cells to mouse chimeras in a. More than half of ViPS cells displayed a restricted contribution to the chimeric heart—a pattern of contribution that was not observed with TiPS cells. FIG. 4E shows the percent of beating EBs (number of beating EBs/total number of EBs) from ES, ViPS cells, and TiPS cells on EB day 9 to 17. The percent of beating EBs peaks around day 10 and then trend down slowly as the dish becomes confluent. FIG. 4F shows results from quantitative RT-PCR gene expression analysis of cTnT in EBs along the time course of EB differentiation. RNAs from three lines of each cell type were pooled for analysis. The level of cTnT was normalized with that of α-actin and the level in day 2 EBs of ES cell lines was set as one. The error bars represent standard deviations of the PCR triplicates. FIG. 4G shows the frequency of cTnT⁺ cells generated from EBs derived from ES cells, ViPS cells, and TiPS cells, as revealed by intracellular FACS analysis with an antibody specific for cTnT. Upper panel shows a representative FACS analysis of cells dissociated on EB day 14 from ES cells, ViPS cells, and TiPS cells. Bar graph displays cells were assayed on EB day 7 and EB day 14 (lower panel). Asterisk denotes significantly different from ES (n=6) cell lines, P<0.01; pound sign denotes significantly different from TiPS (n=22) cell lines, P<0.01. n=23 for ViPS cell lines. Bars represent standard deviation. FIG. 4H shows an improved differentiation protocol dramatically increased the yield of cardiomyocytes from ViPS cells, with a highest 42% cells positive for cardiac Troponin T (cTnT), while the yield of cardiomyocytes from ES and TiPS cells remained relatively low.

FIG. 5 shows a schematic illustration of differentiation of ES cells or iPS cells into embryoid bodies (EB) formation. ES or iPS cells are dissociated into single cells and cultured in hang-drops from the bottom of flipped petri dishes, with about 500 cells/drop. In two days, the cells will aggregate to form EBs. The Petri dishes are flipped back and the EBs are cultured with more medium. The EBs will grow and many developmental processes, including cardiomyogenesis, will be initiated. Cardiomyogenic potential can be assessed by counting the number of cardiac Troponin T positive (cTnT+) cardiomyocytes.

FIGS. 6A-6B shows functional cardiomyocytes differentiated from ViPS cells and are snapshots of a movie to show functional cardiomyocytes differentiated from ViPS. FIG. 6A is from a movie to show that ViPS spontaneously differentiate along cardiac lineages to form beating embryoid bodies (EB) at day 7 (D7). On day 7, over 90% of the EBs were beating. FIG. 6B is from a movie to show that some cells from the EBs ViPS spontaneously differentiate along cardiac lineages to adhere to the flat surface to organize themselves to form beating sheets. In contrast EBs from TiPS or EBs from ES cells were dormant, and did not beat until at least day 8, demonstrating the that ViPS have an earlier initiation of a cardiomyogenic program and a more robust cardiac differentiation program which is advantageous for manufacture of iPS cell-derived cardiomyocytes as well as for efficient and safe scale up.

FIGS. 7A-7D show a higher cardiomyogenic potential of ViPS cells than that of ES cells and TiPS cells. FIG. 7A shows intracellular FACS analysis of embryoid bodies (EBs) to score the percentage of cardiac Troponin T positive (cTnT+) cells. FIG. 7B shows representative intracellular FACS results. FIG. 7C shows a higher expression of most examined cardiogenic genes in day 7 EBs from the ViPS cells. FIG. 7D shows a preferentially higher expression of Mlc2v (ventricular specific) over cTnT (pan-cardiac) in day 7 EBs from the ViPS cells than the ES cells.

FIG. 8 shows a high yield of cardiomyocytes can be reached from ViPS cells. When day 7 EBs from ViPS cells were dissociated and cultured on gelatin with a medium specialized for cardiomyocytes, almost 33% of the cells were cTnT+ cardiomyocytes, as indicated by the intracellular FACS analysis. Control is the immunostaining control where primary antibody was not added.

FIG. 9 shows one embodiment of a strategy to generate human secondary iPS cells from cardiomyocytes. Human fetal cardiac fibroblasts were used to generate the primary iPS cells. Doxycycline inducible reprogramming transgenes were introduced into the fibroblasts by lentiviral transduction, and the cells were cultured with doxycycline until primary iPS cells colonies emerged. The primary iPS cell colonies were selected and passaged for at least three times to establish stable cell lines. A lentivirus will be used to introduce cTnT-eGFP transgene into the primary iPS cells to label cardiomyocytes, thereby enabling selection of cardiac cells differentiated from the primary iPS cells. The transgenic cells will be differentiated for the production of cardiomyocytes which will express GFP and be eGPF positive. The cardiomyocytes, in parallel with fibroblasts, will be isolated and plated on MEF feeder. Doxycycline will be added to the culture medium to induce the expression of the reprogramming factors. Secondary iPS cell colonies will emerge in about 4 weeks. The 2iPS cell colonies will be picked to establish stable cell lines. The cardiomyogenic potential of the cells will be evaluated.

FIG. 10 shows the generation of mouse iPS cells from the tail tip and cardiac fibroblasts with three reprogramming factors, Klf4, Oct3/4 and Sox2. Either tail tip fibroblasts (TTF) or cardiac fibroblasts were obtained from a transgenic mouse that is Rosa26^{YFP/+,} TgMef2c-AHF-dsRed that express YFP when cells have ventricular cardiomyocyte lineage was used and used as somatic cells for reprogramming to iPS cells. The cardiac fibroblasts were obtained by dissociating a heart and plating on uncoated plates, where cardiac fibroblasts attach first to the surface of the plate. The plate is turned upside down and the endothelial cells and cardiac cells are discarded, and the cardiac fibroblasts adhered to the plates collected. Cardiac fibroblasts can be distinguished from endothelial cells or other cardiac cells by ordinary skill in the art as cardiac fibroblasts exhibit a flat morphology of a pattern typical of a plated fibroblast.

FIG. 11A-11B shows Mef2c-AHF-DsRed as a marker of anterior heart field (AHF) derived cardiac progenitors and cardiomyocytes. FIG. 11A shows DsRed expression in the anterior heart field (AHF) which develops into ventricles, aorta and pulmonary tract, at E12.5 days, and expression continuing at P0 and 2 months. FIG. 11B is a schematic of the Mef2c-AHF enhancer which is operatively linked to a Ds-Red reporter marker gene.

FIG. 12 shows fibroblasts from tail tip and cardiac fibroblasts have the same morphology and iPS cells from Tail tip fibroblasts (iPS-TTF) or from cardiac fibroblasts (iPS-CF) has similar clonal morphology and alkaline phosphatase activity as ES cells.

FIG. 13A-13E shows cardiomyocytes derived from ViPS cells display a ventricular phenotype. FIG. 13A shows cTnT immunostaining of cardiomyocytes derived from ES, ViPS, and TiPS cells. Nuclei were stained with DAPI. Note the striated pattern of cTnT protein typical of cardiomyocytes (insets). Scale bar, 10 μm. FIG. 13B shows double immunostaining for cTnI and Mlc2v from cells derived from ES cells, ViPS cells, and TiPS cells. Scale bar, 10 μm. FIG. 13C shows an increased percent of ventricular myocytes (Mlc2v⁺) among all cardiomyocytes (cTnI) from ViPS cells. Asterisk denotes significantly different from ES cell lines (n=6) P<0.05; pound sign denotes significantly different from TiPS cell lines (n=6) P<0.05. n=6 for ViPS cell lines. Bars represent standard deviation. FIG. 13D shows results from gene expression analysis of the FACS-purified cTnT fraction demonstrates increased expression of Mlc2v relative to cTnT. Asterisk denotes significantly different from ES cell lines (n=6) P<0.05; pound sign denotes significantly different from TiPS cell lines (n=6) P<0.05. n=6 for ViPS cell lines. Bars represent standard deviation. FIG. 13E shows a representative action potential recorded from a spontaneously contracting cardiomyocyte derived from ViPS cells.

FIGS. 14A-14E show the biological and molecular mechanism of the ventriculogenic bias in ViPS cells. FIG. 14A shows flow cytometric plots showing increased numbers of Mef2c-AHF-dsRed+ cells upon differentiation of ViPS cells in day 7 EBs. In order to avoid spectral overlap between the dsRed and the YFP signals, we fixed the cells and performed intracellular staining to label the dsRed protein with APC (FL4). FIG. 14B shows increased numbers of Mef2c-AHF-dsRed⁺ cells from ViPS cells. Bar graph displaying percents of dsRed⁺ cells from ViPS cells. Asterisk denotes significantly different from ES cell lines (n=6) P<0.05; pound sign denotes significantly different from TiPS cell lines (n=6) P<0.05. n=6 for ViPS cell lines. Bars represent standard deviations. FIG. 14C shows quantitative RT-PCR results demonstrating gene expression of Nkx2.5 and Isl1 in EBs derived from ES cells, ViPS cells and TiPS cells. RNAs from three lines of each cell type were pooled for analysis. The level of Nkx2.5 or Isl1 was normalized to that of _-actin and the level in day 2 EBs of ES cell lines was set as one for relative comparison. The error bars represent standard deviations of the PCR triplicates. FIG. 14D shows bisulfite sequencing of the promoter regions of Nkx2.5, My12 and Nanog in ES cells, ViPS cells and TiPS cells at passage number eight. The lines represent the genomic regions and the black squares before the arrows indicate exon 1. The lines over the squares demonstrate the promoter regions that were subjected to bisulfite sequencing. Open circles indicate unmethylated CpG dinucleotides, while closed circles indicate methylated CpGs. The percents besides each panel are of the methylated CpGs. FIG. 14E shows a odel depicting that the increased cardiomyogenic potential of ViPS cells is achieved by increased numbers of cardiac progenitors. The functional differences between ViPS cells and TiPS cells are carried throughout differentiation to further bias the cardiomyocytes to display a ventricular-like phenotype. Red, ventricular myocytes; yellow, atrial myocytes.

FIG. 15 shows characterization of secondary iPS cells. Quantitative RT-PCR of a ViPS cell line and a TiPS cell line demonstrate levels of gene expression of Nanog, Pou5f1, and Sox2 comparable to that observed in mouse ES cells. No Nanog, Pou5f1, and Sox2 gene expression was detected in the parent ventricular myocytes and tail tip fibroblasts that were used to generate the secondary iPS cells. Secondary ViPS and TiPS cell colonies express the pluripotency markers SSEA1, Nanog, and Oct3/4 (data not shown).

FIG. 16A-16B shows that secondary iPS cells generate well-differentiated teratoma-like masses containing all three embryonic germ layers when injected into the kidney capsules of immunodeficient mice. FIG. 16A shows NOD/SCID immunodeficient mouse recipients were grafted with three to five day 7 embryoid bodies derived from mouse ES cells, ViPS cells, or TiPS cells. After four weeks, dissection of animals revealed the presence of renal teratoma-like masses. Photographs show representative gross specimens from ES cells, ViPS cells, and TiPS cells. Scale bar, 10 mm. FIG. 16B shows histological examination revealed the presence of various tissues including keratin-containing epidermal tissues (ectoderm), cartilage (mesoderm), and gut-like epithelium (endoderm). Scale bar, 100 μm.

FIG. 17A-17B shows expression analysis through quantitative RT-PCR of selected cardiac genes in EBs. FIG. 17A shows Day 7 EB expression of selected cardiogenic transcription factors (left panel) and marker genes (dsRed & cTnT, right panel). FIG. 17B show day 14 EB expression of selected cardiogenic transcription factors (left panel) and marker genes (dsRed & cTnT, right panel). The expression level of each individual cell line was examined with whole EB RNA, and five lines were included for each cell type. Displayed are the mean levels of five lines. Expression level was normalized to that of β-actin, and the mean level of ES cell lines was set as one for relative comparison. Asterisk denotes significantly different from ES cell lines (n=5) P<0.05, t-test; pound sign denotes significantly different from TiPS cell lines (n=5) P<0.05, t-test. n=5 for ViPS cell lines. Bars represent standard deviations.

FIG. 18A-18B shows characterization of atrial myocytes and ventricular myocytes derived from postnatal pups. FIG. 18A shows immunostaining with cTnI stains cardiomyocytes from both the atria and the ventricles. Mlc2v stains cardiomyocytes from the ventricles but not the atria. Scale bar, 10 μm. AM: atrial myocyte; VM: ventricular myocyte. FIG. 18B shows a representative action potential recorded from a spontaneously contracting cardiomyocyte isolated from the atria (left panel) and from the ventricles (right panel) of postnatal pups.

FIG. 19A-19B shows in vitro differentiation potentials of ViPS cells, TiPS cells, and ES cells at low to high passage numbers. FIG. 19A shows at passage 24, the enhanced cardiomyogenic potential of ViPS cells compared to ES cells and TiPS remains although the absolute difference is diminished compared to cells at lower passage number. Asterisk denotes significantly different from ES cell lines (n=6) P<0.05, t-test; pound sign denotes significantly different from TiPS cell lines (n=6) P<0.05, t-test. n=6 for ViPS cell lines. FIG. 19B shows a representative FACS plot showing an increased percentage of cTnT⁺ cells in cells differentiated from ViPS compared to ES cells and TiPS cells at passage 24.

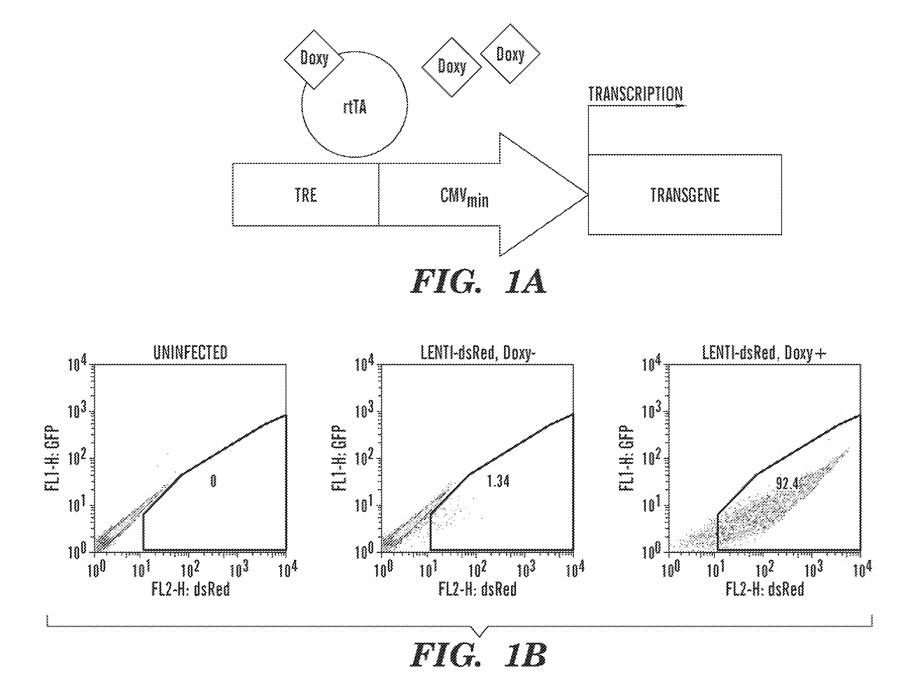

### DETAILED DESCRIPTION OF THE INVENTION

The present invention is related to the generation of ventricular cardiomyocytes, and in particular to generation of ventricular cardiomyocytes from iPS cells. In some embodiments, ventricular cardiomyocytes are differentiated from secondary iPS (2iPS) cell lines, where the secondary iPS (2iPS) cell lines have been generated from reprogramming a cardiac somatic cell which has been differentiated from a primary iPS cell line.

Methods to reprogram a somatic cell to an induced pluripotent stem (iPS) cell by forced expression of a small set of transcription factors results in an iPS cell that resembles an embryonic stem (ES) cells in many aspects. However, despite their great regenerative potential, iPS cells and ES cells have a low efficiency to differentiate to cardiomyocytes. There have been reported significant differences between the iPS cells from different origins, indicating that iPS cells with a cardiac origin might have a higher cardiomyogenic potential than non-cardiac iPS cells or ES cells.

The inventors herein demonstrate and have generated genetically matched iPS cells from mouse ventricular myocytes, termed herein as “ViPS” cells and tail tip fibroblasts, termed herein as “TiPS” cells through an inducible secondary iPS (2iPS) cell system.

The inventors demonstrate that surprisingly, when differentiated through embryoid body (EB) formation, ViPS cells initiated an earlier and more robust cardiomyogenic program, and yielded more cardiomyocytes than TiPS and ES cells. Further more, there is surprisingly a preferentially higher expression of Mlc2v (ventricular specific marker) over cardiac Troponin T (pan-cardiac marker) in the EBs from ViPS cells than ES cells, which suggests that more cardiomyocytes from ViPS are of a ventricular identity than those from ES cells. The inventors have demonstrated a higher ventricular myogenic potential of the ViPS cells, demonstrating that their human counterparts are useful as good source for autologous ventricular myocytes, which can be used extensively in drug discovery, disease modeling and eventually cell-based therapy.

By using a doxycycline inducible system, mouse ventricular induced pluripotent stem cells (hereafter referred to as ViPS cells) with higher cardiomyogenic potential have been generated from ventricular cardiomyocytes. These cells can be used to generate high-quality autologous ventricular cardiomyocytes with high yield.

Another aspect of the present invention relate to the use of human ViPS cells to generate high-quality autologous ventricular cardiomyocytes with high yield to test the efficacy and toxicity of drugs.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “cardiac somatic cell” are used interchangeably herein, refers to a differenated cell located in the heart, and includes without limitation, cardiac cells, cardiovascular cells and other cells of the cardio-vascular system.

The term “cardiovascular stem cell” and “cardiac stem cell” are used interchangeably herein, refers to a stem cell which is capable of proliferation and giving rise to more progenitor cells having the ability to generate a large number of mother cells that can in turn give rise to differentiated, or differentiable daughter cells which can eventually terminally differentiate into cardiac cells, cardiovascular cells and other cells of the cardio-vascular system.

As used herein, the term “stem cells” is used in a broad sense and includes traditional stem cells, progenitor cells, pre-progenitor cells, reserve cells, and the like. The term “stem cell” or “progenitor” are used interchangeably herein, and refer to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation”.

Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including method for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735. Descriptions of stromal cells, including methods for isolating them, may be found in, among other places, Prockop, Science, 276:7174, 1997; Theise et al., Hepatology, 31:235 40, 2000; Current Protocols in Cell Biology, Bonifacino et al., eds., John Wiley & Sons, 2000 (including updates through March, 2002); and U.S. Pat. No. 4,963,489. The skilled artisan will understand that the stem cells and/or stromal cells selected for inclusion in a transplant with mixed SVF cells or SVF-matrix construct (e.g. for encapsulating a tissue or cell transplant according to the constructs and methods as disclosed herein) are typically appropriate for the intended use of that construct.

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

As indicated above, there are different levels or classes of cells falling under the general definition of a “stem cell.” These are “totipotent,” “pluripotent” and “multipotent” stem cells. The term “totipotent” refers to a stem cell that can give rise to any tissue or cell type in the body. “Pluripotent” stem cells can give rise to any type of cell in the body except germ line cells. Stem cells that can give rise to a smaller or limited number of different cell types are generally termed “multipotent.” Thus, totipotent cells differentiate into pluripotent cells that can give rise to most, but not all, of the tissues necessary for fetal development. Pluripotent cells undergo further differentiation into multipotent cells that are committed to give rise to cells that have a particular function. For example, multipotent hematopoietic stem cells give rise to the red blood cells, white blood cells and platelets in the blood.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratomas formation assay. Pluripotency is also evidenced by the expression of embryonic stem (ES) cell markers, although the preferred test for pluripotency is the demonstration of the capacity to differentiate into cells of each of the three germ layers. In some embodiments, a pluripotent cell is an undifferentiated cell.

The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of multipotent cells include adult stem cells, such as for example, hematopoietic stem cells and neural stem cells. Multipotent means a stem cell may form many types of cells in a given lineage, but not cells of other lineages. For example, a multipotent blood stem cell can form the many different types of blood cells (red, white, platelets, etc. . . . ), but it cannot form neurons.

The term “multipotency” refers to a cell with the degree of developmental versatility that is less than totipotent and pluripotent.

The term “totipotency” refers to a cell with the degree of differentiation describing a capacity to make all of the cells in the adult body as well as the extra-embryonic tissues including the placenta. The fertilized egg (zygote) is totipotent as are the early cleaved cells (blastomeres)

The term “embryonic stem cell” or “ES cell” or “ESC” are used interchangeably herein and refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like. In some embodiments, an ES cell can be obtained without destroying the embryo, for example, without destroying a human embryo.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to a process that alters or reverses the differentiation state of a differentiated cell (e.g. a somatic cell). Stated another way, reprogramming refers to a process of driving the differentiation of a cell backwards to a more undifferentiated or more primitive type of cell. The cell to be reprogrammed can be either partially or terminally differentiated prior to reprogramming. In some embodiments, reprogramming encompasses complete reversion of the differentiation state of a differentiated cell (e.g. a somatic cell) to a pluripotent state. In some embodiments, reprogramming also encompasses partial reversion of the differentiation state of a differentiated cell (e.g. a somatic cell) to a multipotent state. In some embodiments, reprogramming encompasses complete or partial reversion of the differentiation state of a differentiated cell (e.g. a somatic cell) to an undifferentiated cell. Reprogramming also encompasses partial reversion of the differentiation state of a somatic cell to a state that renders the cell more susceptible to complete reprogramming to a pluripotent state when subjected to additional manipulations such as those described herein. Such contacting may result in expression of particular genes by the cells, which expression contributes to reprogramming. In certain embodiments of the invention, reprogramming of a differentiated cell (e.g. a somatic cell) causes the differentiated cell to assume an undifferentiated state (e.g. is an undifferentiated cell). In some embodiments, reprogramming of a differentiated cell (e.g. a somatic cell) causes the differentiated cell to assume a pluripotent-like state. The resulting cells are referred to herein as “iPS cells”, or “secondary iPS cells” where the somatic cell which was reprogrammed was differentiated from a primary iPS cell.

Reprogramming involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods of the invention may also be of use for such purposes. Certain of the compositions and methods of the present invention contribute to establishing the pluripotent state. The methods may be practiced on cells that fully differentiated and/or restricted to giving rise only to cells of that particular type, rather than on cells that are already multipotent or pluripotent.

The term “reprogrammed cell” as used herein refers to a cell which has been reprogrammed from a differentiated cell according to the methods as disclosed herein. In some embodiments, a reprogrammed cell is a cell which has undergone epigenetic reprogramming. The term “reprogrammed cell” encompasses an undifferentiated cell. The term “reprogrammed cell” also includes a partially reprogrammed cell except where it specifically indicates it does not include a partially reprogrammed cell.

The term “partially reprogrammed cell” as referred to herein refers to a cell which has been reprogrammed from a differentiated cell, by the methods as disclosed herein, wherein the partially reprogrammed cell has not been completely reprogrammed to pluripotent state but rather to a non-pluripotent stable intermediate state. In some embodiments, a partially reprogrammed cell expresses at least one or at least two or at least three but not all of the following markers; alkaline phosphatase (AP), NANOG, OCT-4, SOX-2, SSEA4, TRA-1-60 or TRA-1-81. In some embodiments, a partially reprogrammed cell expresses markers from one or two germ cell layers or all three embryonic germ layers. Markers of endoderm cells include, Gata4, FoxA2, PDX1, Noda1, Sox7 and Sox17. Markers of mesoderm cells include, Brachyury, GSC, LEF1, Mox1 and Tie1. Markers of ectoderm cells include criptol, EN1, GFAP, Islet 1, LIM1 and Nestin. In some embodiments, a partially reprogrammed cell is an undifferentiated cell. In some embodiments, the methods as disclosed herein can be used to generate a partially reprogrammed cell (or population thereof) by contacting a differentiated cell with any compound selected from compounds of Formulas I-XI which replace one or two of the following reprogramming genes selected from the group of; Sox2, Oct3/4 or Klf4.

The term a “reprogramming gene”, as used herein, refers to a gene whose expression, contributes to the reprogramming of a differentiated cell, e.g. a somatic cell to an undifferentiated cell, e.g. a cell of a pluripotent state or partially pluripotent state. A reprogramming gene can be, for example, genes encoding transcription factors Sox2, Oct3/4, Klf4, Nanog, Lin-38, c-myc and the like.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for reprogramming a differentiated cell can be performed both in vivo and in vitro (where in vivo is practiced when a differentiated cell is present within a subject, and where in vitro is practiced using isolated differentiated cell maintained in culture). In some embodiments, where a differentiated cell or population of differentiated cells are cultured in vitro, the differentiated cell can be cultured in an organotypic slice culture, such as described in, e.g., meneghel-Rozzo et al., (2004), Cell Tissue Res, 316(3); 295-303, which is incorporated herein in its entirety by reference.

As used herein, the terms “iPS cell” and “induced pluripotent stem cell” are used interchangeably and refers to a pluripotent cell artificially derived (e.g., induced by complete or partial reversal) from a differentiated cell (e.g. a non-pluripotent cell) or a somatic cell such as a differentiated somatic cell. iPS cells are capable of self-renewal and differentiation into cell fate-committed stem cells, including neural stem cells, as well as various types of mature cells.

The term “primary iPS cell” refers to a pluripotent cell artificially derived from reprogramming of a differentiated somatic cell where the differentiated somatic cell was not differentiated from an iPS cell.

The term “secondary iPS cell” or “2iPS cell” are used interchangeably and refers to a pluripotent cell artificially derived from reprogramming of a differentiated somatic cell where the differentiated somatic cell was differentiated from an iPS cell.

The term “derived from” used in the context of a cell derived from another cell means that a cell has stemmed (e.g. changed from or produced by) a cell which is a different cell type. In some instances, for e.g. a cell derived from an iPS cell refers to a cell which has differentiated from an iPS cell. Alternatively, a cell can be converted from one cell type to a different cell type by a process referred to as transdifferention or direct reprogramming. Alternatively, in the terms of iPS cells, a cell (e.g. iPS cell) can be derived from a differentiated cell by a process referred to in the art as dedifferentiation or reprogramming. The term “derived from” also refers to cells which have been differentiated from a progenitor cell.

When used in connection with cell cultures and/or cell populations, the term “portion” means any non-zero amount of the cell culture or cell population, which ranges from a single cell to the entirety of the cell culture or cells population. In some embodiments, the term “portion” means at least about 0.5% or at last about 1% or at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94% or at least 95% of the cell culture or cell population.

With respect to cells in cell cultures or in cell populations, the term “substantially free of” means that the specified cell type of which the cell culture or cell population is free, is present in an amount of less than about 10%, less than about 9%, less than about 8%, less than about 7%, less than about 6%, less than about 5%, less than about 4%, less than about 3%, less than about 2% or less than about 1% of the total number of cells present in the cell culture or cell population.

As used herein, “exogenously added,” compounds such as growth factors, differentiation factors, and the like, in the context of cultures or conditioned media, refers to growth factors that are added to the cultures or media to supplement any compounds or growth factors that may already be present in the culture or media. For example, in some embodiments, of the present invention, cells cultures and or cell populations do not include an exogenously-added retinoid.

As used herein, “produced from hESCs,” “derived from hESCs,” “differentiated from hESCs” and equivalent expressions refer to the production of a differentiated cell type from hESCs in vitro rather than in vivo.

As used herein, cardiac cells or ventricular cardiomyocytes (VM) “produced from ViPS cells” or “differentiated from ViPS cells” and equivalent expressions refer to the production of a differentiated cell type from secondary iPS cell, where the secondary iPS cell was derived from reprogramming a cardiac somatic cell, e.g., a ventricular cardiomyocytes or cardiac fibroblast which has been differentiated from an iPS cell. Stated another way, the reprogramming of a somatic cell produces a primary iPSC, which can be differentiated into a cardiac somatic cell e.g., a ventricular cardiomyocytes or cardiac fibroblast. Such cardiac somatic cells (differentiated from the primary iPSC) can be reprogrammed into secondary iPSC, which can then be subsequently differentiated into cardiac somatic cells e.g., a ventricular cardiomyocytes or cardiac fibroblast, referred to as “cardiac cells or ventricular cardiomyocytes (VM) produced from ViPS cells” As used herein, the term “adult cell” refers to a cell found throughout the body after embryonic development.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

The term “differentiation” in the present context means the formation of cells expressing markers known to be associated with cells that are more specialized and closer to becoming terminally differentiated cells incapable of further differentiation. The pathway along which cells progress from a less committed cell, to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated. Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, we note that in the context of this specification, the terms “differentiation” or “differentiated” refer to cells that are more specialized in their fate or function than at a previous point in their development, and includes both cells that are terminally differentiated and cells that, although not terminally differentiated, are more specialized than at a previous point in their development. The development of a cell from an uncommitted cell (for example, a stem cell), to a cell with an increasing degree of commitment to a particular differentiated cell type, and finally to a terminally differentiated cell is known as progressive differentiation or progressive commitment. A cell that is “differentiated” relative to a progenitor cell has one or more phenotypic differences relative to that progenitor cell. Phenotypic differences include, but are not limited to morphologic differences and differences in gene expression and biological activity, including not only the presence or absence of an expressed marker, but also differences in the amount of a marker and differences in the co-expression patterns of a set of markers.

The term “differentiation” as used herein refers to the cellular development of a cell from a primitive stage towards a more mature (i.e. less primitive) cell.

The term “directed differentiation” as used herein refers to forcing differentiation of a cell from an undifferentiated (e.g. more primitive cell) to a more mature cell type (i.e. less primitive cell) via genetic and/or environmental manipulation. In some embodiments, a primary iPS cell can be directed to be differentiated into specific cardiac cell types, such as cardiomyocytes, ventricular cardiomyocytes, vascular lineages and muscle cell types and the like.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “regeneration” means regrowth of a cell population, organ or tissue after disease or trauma.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, and refers to a process of a cell making more copies of itself (e.g. duplication) of the cell. In some embodiments, reprogrammed cells are capable of renewal of themselves by dividing into the same undifferentiated cells (e.g. pluripotent or non-specialized cell type) over long periods, and/or many months to years. In some instances, proliferation refers to the expansion of reprogrammed cells by the repeated division of single cells into two identical daughter cells.

The term “lineages” as used herein refers to a term to describe cells with a common ancestry or cells with a common developmental fate, for example cells that are derived from iPS-VM or ViPS cellshave a developmental fate to develop into ventricular cardiomyocytes.

As used herein, the term “clonal cell line” refers to a cell lineage that can be maintained in culture and has the potential to propagate indefinitely. A clonal cell line can be a stem cell line or be derived from a stem cell, and where the clonal cell line is used in the context of a clonal cell line comprising stem cells, the term refers to stem cells which have been cultured under in vitro conditions that allow proliferation without differentiation for months to years. Such clonal stem cell lines can have the potential to differentiate along several lineages of the cells from the original stem cell.

The term “media” as referred to herein is a medium for maintaining a tissue or cell population, or culturing a cell population (e.g. “culture media”) containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

By “conditioned medium” is meant, a medium that is altered as compared to a base medium. For example, the conditioning of a medium may include molecules, such as nutrients and/or growth factors, e.g., VEGF or TGFβ inhibitors such as ALK5i, to be added to or depleted from the original levels found in the base medium. In some embodiments, a medium is conditioned by allowing cells of certain types to be grown or maintained in the medium under certain conditions for a certain period of time. For example, a medium can be conditioned by allowing ViPS cells to be expanded, differentiated along cardiac lineages or maintained in a medium of defined composition at a defined temperature for a defined number of hours. As will be appreciated by those of skill in the art, numerous combinations of cells, media types, durations and environmental conditions can be used to produce nearly an infinite array of conditioned media.

The term “phenotype” refers to one or a number of total biological characteristics that define the cell or organism under a particular set of environmental conditions and factors, regardless of the actual genotype.

A “marker” as used herein describes the characteristics and/or phenotype of a cell. Markers can be used for selection of cells comprising characteristics of interest. Markers will vary with specific cells. Markers are characteristics, whether morphological, functional or biochemical (enzymatic) characteristics particular to a cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

The terms “mesenchymal cell” or “mesenchyme” are used interchangeably herein and refer in some instances to the fusiform or satellite cells found between the ectoderm and endoderm of young embryos; most mesenchymal cells are derived from established mesodermal layers, but in the cephalic region they also develop from neural crest or neural tube ectoderm. Mesenchymal cells have a pluripotent capacity, particularly embryonic mesenchymal cells in the embryonic body, developing at different locations into any of the types of connective or supporting tissues, to smooth muscle, to vascular endothelium, and to blood cells.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from. In some embodiments, the isolated population is an isolated population of reprogrammed cells which is a substantially pure population of reprogrammed cells as compared to a heterogeneous population of cells comprising reprogrammed cells and cells from which the reprogrammed cells were derived.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of ViPS or ventricular cardiomyocytes (VM), refers to a population of ViPS or ventricular cardiomyocytes (VM) respectively that contain fewer than about 20%, more preferably fewer than about 15%, 10%, 8%, 7%, most preferably fewer than about 5%, 4%, 3%, 2%, 1%, or less than 1%, of cells that are not ViPS or ventricular cardiomyocytes (VM), respectively, or their progeny as defined by the terms herein. In some embodiments, the present invention encompasses methods to expand a population ViPS, where the expanded population of ViPS can be used to differentiate into ventricular cardiomyocytes, which is a substantially pure population of ViPS derived ventricular cardiomyocytes.

The term “functional assay” as used herein is a test which assesses the properties of a cell, such as a cell's gene expression or developmental state by evaluating its growth or ability to live under certain circumstances. In some embodiments, a ViPS cell can be used in a functional assay to determine the development and function into cardiac lineages, e.g., ventricular cardiomyocytes in the presence of agents. In some embodiments, ventricular cardiomyocytes differentiated from ViPS cells can be used in a functional assay to determine the effect of toxins or agents or other compounds on the function, e.g., contractile force of ventricular cardiomyocytes.

The term “disease model” as used herein refers to the use of laboratory cell culture or animal research to obtain new information about human disease or illness. In some embodiments, a population of ventricular cardiomyocytes differentiated from ViPS cells produced by the methods as disclosed herein can be used in disease modeling experiments.

The term “drug screening” as used herein refers to the use of cells, e.g., ventricular cardiomyocytes differentiated from ViPS (e.g., 2iPS-VM) cells and tissues derived therefrom in the laboratory to identify drugs with a specific function. In some embodiments, the present invention provides drug screening methods of differentiated cells to identify compounds or drugs which increase or decrease the contractile force of e.g., ventricular cardiomyocytes differentiated from ViPS cells. In some embodiments, the present invention provides drug screening methods of ViPS cells to identify compounds or drugs which increase the differentiation into ventricular cardiomyocytes. In alternative embodiments, the present invention provides drug screening on ventricular cardiomyocytes differentiated from ViPS, where the ViPS are of human origin to identify compounds or drugs useful as therapies for diseases or illnesses (e.g. human cardiovascular diseases, disorders or illnesses), for example where the human ViPS carry a specific genetic mutation or polymorphism resulting or contributing to a cardiovascular disease or disorder.

The terms “subject” and “individual” are used interchangeably herein, and refer to an animal, for example a human, to whom cardiomyocytes differentiated from ViPS as disclosed herein can be implanted into, for e.g. treatment, which in some embodiments encompasses prophylactic treatment or for a disease model, with methods and compositions described herein, is or are provided. For treatment of disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like are also encompassed in the term subject.

The term “tissue” refers to a group or layer of similarly specialized cells which together perform certain special functions. The term “tissue-specific” refers to a source or defining characteristic of cells from a specific tissue.

As used herein, the term “donor” refers to a subject to which a organ, tissue or cell to be transplanted is harvested from.

As used herein, the term “recipient” refers to a subject which will receive a transplanted organ, tissue or cell.

The term “graft” as used herein refers to the process whereby a free (unattached) cell, tissue, or organ integrates into a tissue following transplantation into a subject.

The term “allograft” refers to a transplanted cell, tissue, or organ derived from a different animal of the same species.

The term “xenograft” or “xenotransplant” as used herein refers to a transplanted cell, tissue, or organ derived from an animal of a different species. In some embodiments, a xenograft is a surgical graft of tissue from one species to an unlike species, genus or family. By way of an example, a graft from a baboon to a human is a xenograft.

The term “xenotransplantation” refers to the process of transplantation of living cells, tissues or organs from one species to another, such as from pigs to humans.

The term “three-dimensional matrix” or “scaffold” or “matrices” as used herein refers in the broad sense to a composition comprising a biocompatible matrix, scaffold, or the like. The three-dimensional matrix may be liquid, gel, semi-solid, or solid at 25° C. The three-dimensional matrix may be biodegradable or non-biodegradable. In some embodiments, the three-dimensional matrix is biocompatible, or bioresorbable or bioreplacable. Exemplary three-dimensional matrices include polymers and hydrogels comprising collagen, fibrin, chitosan, MATRIGEL™, polyethylene glycol, dextrans including chemically crosslinkable or photocrosslinkable dextrans, processed tissue matrix such as submucosal tissue and the like. In certain embodiments, the three-dimensional matrix comprises allogeneic components, autologous components, or both allogeneic components and autologous components. In certain embodiments, the three-dimensional matrix comprises synthetic or semi-synthetic materials. In certain embodiments, the three-dimensional matrix comprises a framework or support, such as a fibrin-derived scaffold.

The term “biodegradable” as used herein denotes a composition that is not biologically harmful and can be chemically degraded or decomposed by natural effectors (e.g., weather, soil bacteria, plants, animals).

The term “bioresorbable” refers to the ability of a material to be reabsorbed over time in the body (e.g. in vivo) so that its original presence is no longer detected once it has been reabsorbed.

The term “bioreplaceable” as used herein, and when used in the context of an implant, refers to a process where de novo growth of the endogenous tissue replaces the implant material. A bioreplacable material as disclosed herein does not provoke an immune or inflammatory response from the subject and does not induce fibrosis. A bioreplaceable material is distinguished from bioresorbable material in that bioresorbable material is not replaced by de novo growth by endogenous tissue.

The term “contacting” or “contact” as used herein as in connection with contacting a primary iPS cell with Dox or another agent to induce expression of the reprogramming factors, can comprise administering an agent, optionally in a composition, to a culture media which is used to culture a population of primary iPS cells.

As used herein, the terms “administering,” “introducing” and “transplanting” are used interchangeably and refer to the placement of the cardiomyocytes differentiated from ViPS as described herein into a subject by a method or route which results in at least partial localization of the human cardiomyocytes differentiated from ViPS at a desired site. The human cardiomyocytes differentiated from ViPS can be administered by any appropriate route which results in delivery to a desired location in the subject where at least a portion of the human cardiomyocytes differentiated from ViPS remain viable. The period of viability of the cells after administration to a subject can be as short as a few hours, e.g. twenty-four hours, to a few days, to as long as several years.

The term “modulate” is used consistently with its use in the art, e.g., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value. The term “substantially” as used herein means a proportion of at least about 60%, or preferably at least about 70% or at least about 80%, or at least about 90%, at least about 95%, at least about 97% or at least about 99% or more, or any integer between 70% and 100%.

The terms “permeabilize”, “vascularizing”, or “vascularization” as used herein refer to providing a functional or substantially functional vascular network to an organ or tissue, particularly an engineered tissue. A functional or substantially functional vascular network is one that perfuses or is capable of perfusing the tissue or organ to meet some or all of the tissue's or organ's nutritional needs, oxygen demand, and waste product elimination needs. A vascular tissue is a natural tissue that is rich in vascular elements, such as microvessels, for example, but without limitation, adipose tissue.

The terms “revascularize”, “revascularizing”, “neovascularization”, or “revascularization” as used herein refer to revising an existing vascular network or establishing a new functional or substantially functional vascular network in a tissue or organ that has an avascular or hypovascular zone, typically due to disease, congenital defect, or injury. Revascularizing such a tissue or organ may result in restored or augmented function.

The terms “enhance vascularization” as used herein refers to an increase or acceleration in the rate of formation of a vascularized network. In some embodiments, an enhanced vascularization refers to the formation of a more dense capillary or vascularized network as compared to in the absence of the method. Stated another way, an enhancement in vascularization refers to a statistically significant increase in the rate of formation of a vascularized network, or alternatively a statistically significant increase in the amount of capillary which form the vascularized network.

The term “genetically modified” cell, e.g. a genetically modified iPSC or cardiomyocytes differentiated from ViPS cells as used herein refers to a iPSC or cardiomyocytes differentiated from ViPS into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (e.g., sequences naturally found in the cells) but in a non-naturally occurring arrangement (e.g., a coding region linked to a promoter from a different gene), or altered versions of native sequences, etc. The process of transferring the nucleic into the cell is referred to as “transducing a cell” and can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, e.g., “selective conditions”. To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The term “transduction” as used herein refers to the use of viral particles to introduce new genetic material into a cell

The term “transfection” as used herein refers the use of chemical methods, most often lipid containing vesicles, to introduce new genetic material into a cell

The term “transformation” as used herein refers to when a cell becomes functionally abnormal in the process of malignancy, often obtaining a new capacity to multiply indefinitely or under new circumstances.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” or “recombinant gene” refers to a nucleic acid comprising an open reading frame encoding a polypeptide, including both exon and (optionally) intron sequences.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

A “reporter gene” as used herein encompasses any gene that is genetically introduced into a cell that adds to the phenotype of the stem cell. Reporter genes as disclosed in this invention are intended to encompass fluorescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny.

The term “recombinant,” as used herein, means that a protein is derived from a prokaryotic or eukaryotic expression system.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g., EPV and EBV vectors.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome. It goes without saying that an iPS cell generated by a non-integrating viral vector will not be administered to a subject unless it and its progeny are free from viral remnants.

While it is known that some non-integrating vectors integrate into the host genome at extremely low frequencies (i.e., 10⁻⁴ to 10⁻⁵), a non-integrating vector, as the term is used herein, refers to vectors having a frequency of integration of less than 0.1% of the total number of infected cells; preferably the frequency of integration is less than 0.01%, less than 0.001%, less than 0.0001%, or less than 0.000001% (or lower) of the total number of infected cells. In one embodiment, the vector does not integrate at all. In another embodiment, the viral integration remnants of the virus are below the detection threshold as assayed by PCR (for nucleic acid detection) or immunoassay (for protein detection). In general, 2iPS cells produced by the methods described herein should be assayed for an integration event by the viral vector using, for example, PCR-mediated detection of the viral genome prior to administering a population of 2iPS cells to a subject. Any 2iPS cell, or progeny derived therefrom with detectable integration products should not be administered to a subject.

In some embodiments, the vector is a non-viral polycystronic vector as disclosed in Gonzalez et al., Proc. Natl. Acad. Sci. USA 2009 106:8918-8922; Carey et al., PNAS, 2009; 106; 157-162, WO/2009/065618 and WO/2000/071096 and Okita et al., Science 7, 2008: 322; 949-953, which are all incorporated herein in their entirety by reference.

As used herein, the term “viral remnants” refers to any viral protein or nucleic acid sequence introduced using a viral vector. Generally, integrating viral vectors will incorporate their sequence into the genome; such sequences are referred to herein as a “viral integration remnant”. However, the temporary nature of a non-integrating virus means that the expression, and presence of, the virus is temporary and is not passed to daughter cells. Thus, upon passaging of a re-programmed cell the viral remnants of the non-integrating virus are essentially removed.

As used herein, the term “free of viral integration remnants” and “substantially free of viral integration remnants” refers to iPS cells that do not have detectable levels of an integrated adenoviral genome or an adenoviral specific protein product (i.e., a product other than the gene of interest), as assayed by PCR or immunoassay. Thus, the iPS cells that are free (or substantially free) of viral remnants have been cultured for a sufficient period of time that transient expression of the adenoviral vector leaves the cells substantially free of viral remnants.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “regulatory sequence” and “promoter” are used interchangeably herein, refers to a generic term used throughout the specification to refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “disease” or “disorder” is used interchangeably herein, and refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition or affection.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “pathology” as used herein, refers to symptoms, for example, structural and functional changes in a cell, tissue, or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

As used herein, the term “treating” or “treatment” are used interchangeably herein and refers to reducing or decreasing or alleviating or halting at least one adverse effect or symptom of a cardiovascular condition, disease or disorder, i.e., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. In some embodiments, the term “treatment” as used herein refers to prophylactic treatment or preventative treatment to prevent the development of a symptom of a cardiovascular condition in a subject.

Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health. In some embodiments, the term to treat also encompasses preventative measures and/or prophylactic treatment, which includes administering a pharmaceutical composition as disclosed herein to prevent the onset of a disease or disorder.

The term “effective amount” as used herein refers to the amount of therapeutic agent of pharmaceutical composition, e.g., an amount of the synthetic modified RNA to express sufficient amount of the protein to reduce at least one or more symptom(s) of the disease or disorder, and relates to a sufficient amount of pharmacological composition to provide the desired effect. The phrase “therapeutically effective amount” as used herein, e.g., of a synthetic modified RNA as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylatically significant reduction in a symptom or clinical marker associated with a cardiac dysfunction or disorder when administered to a typical subject who has a cardiovascular condition, disease or disorder.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

With reference to the treatment of a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick J P, Am J Physiol Heart circ Physiol 285; H1797; 2003) and animal models acute myocardial infarction. (Yang Z, Am J Physiol Heart Circ. Physiol 282:H949:2002; Guo Y, J Mol Cell Cardiol 33; 825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals. By “earlier” is meant that a decrease, for example in the size of the tumor occurs at least 5% earlier, but preferably more, e.g., one day earlier, two days earlier, 3 days earlier, or more.

As used herein, the term “treating” when used in reference to a treatment of a cardiovascular disease or disorder is used to refer to the reduction of a symptom and/or a biochemical marker of a cardiovascular disease or disorder, for example a reduction in at least one biochemical marker of a cardiovascular disease by at least about 10% would be considered an effective treatment. Examples of such biochemical markers of cardiovascular disease include a reduction of, for example, creatine phosphokinase (CPK), aspartate aminotransferase (AST), lactate dehydrogenase (LDH) in the blood, and/or a decrease in a symptom of cardiovascular disease and/or an improvement in blood flow and cardiac function as determined by someone of ordinary skill in the art as measured by electrocardiogram (ECG or EKG), or echocardiogram (heart ultrasound), Doppler ultrasound and nuclear medicine imaging. A reduction in a symptom of a cardiovascular disease by at least about 10% would also be considered effective treatment by the methods as disclosed herein. As alternative examples, a reduction in a symptom of cardiovascular disease, for example a reduction of at least one of the following; dyspnea, chest pain, palpitations, dizziness, syncope, edema, cyanosis etc. by at least about 10% or a cessation of such systems, or a reduction in the size one such symptom of a cardiovascular disease by at least about 10% would also be considered as affective treatments by the methods as disclosed herein. In some embodiments, it is preferred, but not required that the therapeutic agent actually eliminate the cardiovascular disease or disorder, rather just reduce a symptom to a manageable extent.

Subjects amenable to treatment by the methods as disclosed herein can be identified by any method to diagnose myocardial infarction (commonly referred to as a heart attack) commonly known by persons of ordinary skill in the art are amenable to treatment using the methods as disclosed herein, and such diagnostic methods include, for example but are not limited to; (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. In some embodiments, pharmaceutical compositions can be specifically formulated for direct delivery to a target tissue or organ, for example, by direct injection or via catheter injection to a target tissue. In other embodiments, compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

As used herein, the terms “administering,” “introducing” and “transplanting” are used interchangeably and refer to the placement of a pharmaceutical composition comprising ViPS or cardiac cells differentiated from ViPS, or a composition comprising a population of ViPS and their differentiated progeny as described herein, into a subject by a method or route which results in at least partial localization of the pharmaceutical composition, at a desired site or tissue location. In some embodiments, the pharmaceutical composition comprising ViPS or cardiac cells differentiated from ViPS can be administered by any appropriate route which results in effective treatment in the subject, i.e. administration results in delivery to a desired location or tissue in the subject where at least a portion of the ViPS or cardiac cells differentiated from ViPS is located at a desired target tissue or target cell location.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cardiovascular stem cells and/or their progeny and/or compound and/or other material other than directly into the cardiac tissue, such that it enters the animal's system and, thus, is subject to metabolism and other like processes, for example, subcutaneous or intravenous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The term “drug” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “transplantation” as used herein refers to introduction of new cells (e.g. reprogrammed cells), tissues (such as differentiated cells produced from reprogrammed cells), or organs into a host (i.e. transplant recipient or transplant subject)

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### General

One aspect of the present invention relates to a method of generating an induced pluripotent stem (iPS) cell with a high efficiency to differentiate into a ventricular cardiomyocyte comprising: (i) reprogramming a first cardiac cell by contacting it with at least one reprogramming factor to produce a first induced pluripotent stem cell; (ii) differentiating the first induced pluripotent stem cell along cardiogenic lineages to generate a second cardiac cell; (iii) reprogramming the first cardiac cell by contacting it with at least one reprogramming factor to produce a second induced pluripotent stem cell.

In some embodiments, the first cell is a cardiac cell, e.g., a cardiac fibroblast or a cardiomyocyte, e.g., a ventricular cardiomyocyte. In some embodiments, a first cell can be any cell type, such as a pancreatic cell or a liver cell. In some embodiments, where the first cell is from a particular tissue or cell type, then the primary iPS cell derived from the reprogramming is the first cell is differentiated to lineages of the particular tissue or cell type to generate a second cell of the same tissue type or cell type as the first cell. The second cell type can be reprogrammed to a second iPS cell and then differentiated again to a cell of the same cell type of founding first cell.

For example, in some embodiments, the somatic first cell, e.g., fibroblast, such as skin fibroblast, or a cardiac fibroblast can be reprogrammed into a primary iPS cell, which is differentiated along cardiac lineages to a second somatic cell. Methods to direct the differentiation of iPS cells along cardiac lineages is well known by persons of ordinary skill in the art, and are disclosed in WO2010/00703, WO2009/145761, US2009/0202498, WO2009/097411, WO2009/118928, US2009/0202498 and US2008/0187494, which are incorporated herein in their entirety by reference. For example, a primary iPS cell can be cultured in a culture conditions to enhance differentiation to a specific lineage, e.g, cardiac linages. For example, myocardial lineage differentiation may be promoted by including cardiotrophic agents in the culture, e.g. agents capable of forming high energy phosphate bonds (such as creatine) and acyl group carrier molecules (such as carnitine); and a cardiomyocyte calcium channel modulator (such as taurine). Optionally, cardiotropic factors, including, but not limited to those described in U.S. Patent Application Serial No. 2003/0022367 which is incorporate herein in its entirety by reference, may be added to the culture. Such factors may include, for example but not limited to nucleotide analogs that affect DNA methylation and alter expression of cardiomyocyte-related genes; TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the cripto gene; antibodies, peptidomimetics with agonist activity for the same receptors, pseudo ligands, for example peptides and antibodies, cells secreting such factors, and other methods for directed differentiation of stem cells along specific cell lineages in particular cardiomyocyte lineages.

In some embodiments, the second somatic cell is a cardiac cell, e.g., a ventricular cardiomyocyte. In some embodiments, the second somatic cell, e.g., a cardiac cell, e.g., a ventricular cardiomyocyte is reprogrammed to a secondary iPS (2iPS) cell. In some embodiments, such a secondary iPS (2iPS) cell can spontaneously differentiate into ventricular cardiomyocytes.

In another embodiment, a somatic first cell is a fibroblast or any cell from the pancreas, e.g., a pancreatic cell, e.g., a endocrine cell, or a pancreatic β-cell can be reprogrammed into a primary iPS cell, which is differentiated along pancreatic lineages to a second somatic cell, e.g., a endocrine cell, or a pancreatic β-cell. Methods to direct the differentiation of iPS cells along pancreatic lineages are well known by persons of ordinary skill in the art. Methods to differentiate an iPS along pancreatic lineages are disclosed in WO2005/097977, WO2009/143241, WO2010/051213, WO2010/051223, WO2006/094286, WO2010/051213, WO2007/130474 which are incorporated herein in their entirety by reference. A primary iPS cell can be differentiated into a pancreatic cell linage. The pancreas is composed of exocrine and endocrine compartments. The endocrine compartment consists of islets of Langerhans, clusters of four cell types that synthesize peptide hormones: insulin (beta cells), glucagon (alpha cells), somatostatin (gamma cells), and pancreatic polypeptide (PP cells). Although the adult pancreas and central nervous system (CNS) have distinct origins and functions, similar mechanisms control the development of both organs. Strategies for differentiation along pancreatic lineages include plating primary iPS EBs into a serum-free medium, expansion in the presence of basic fibroblast growth factor (bFGF), followed by mitogen withdrawal to promote cessation of cell division and differentiation. A B27 supplement and nicotinamide may improve the yield of pancreatic endocrine cells. The secondary somatic cells in the pancreatic lineages express GATA-4 and HNF3, as well as markers of pancreatic beta cell fate, including the insulin I, insulin II, islet amyloid polypeptide (IAPP), and the glucose transporter-2 (GLUT 2). Glucagon, a marker for the pancreatic alpha cell, may also induced in differentiated cells. The pancreatic transcription factor PDX-1 is expressed. These ES cell-derived differentiating cells have been shown to self-assemble into structures resembling pancreatic islets both topologically and functionally (Lumelsky et al. (2001) Science 292(5520):1389-94.

In some embodiments, the second somatic cell is a pancreatic cell, e.g., a Pdx+ pancreatic cell or an exocrine cell or a pancreatic β cell, or an insulin producing cell. In some embodiments, the second somatic cell, e.g., a Pdx+ pancreatic cell or an exocrine cell or a pancreatic β cell is reprogrammed to a secondary iPS (2iPS) cell. In some embodiments, such a secondary iPS (2iPS) cell can spontaneously differentiate into Pdx+pancreatic cell, or an exocrine cell or a pancreatic β cell or an insulin producing cell.

As used herein, the term “β-cell” refers to an insulin producing cell of the pancreas or a cell of a pancreatic β-cell (beta cell) phenotype. The phenotype of a pancreatic β-cell is well known by persons of ordinary skill in the art, and include, for example, secretion of insulin in response to an increase in glucose level, expression of markers such as c-peptide, PDX-1 polypeptide and Glut 2, as well as distinct morphological characteristics such as organized in islets in pancreas in vivo, and typically have small spindle like cells of about 9-15 μm diameter.

As used herein, the term “insulin producing cell” includes β-cells as that term is described herein that synthesize (i.e., transcribe the insulin gene, translate the proinsulin mRNA, and modify the proinsulin mRNA into the insulin protein), express (i.e., manifest the phenotypic trait carried by the insulin gene), or secrete (release insulin into the extracellular space) insulin in a constitutive or inducible manner. A population of pancreatic β-cells or insulin producing cells made by the present invention may contain β-cells or β-like cells (e.g., cells that have at least two characteristics of an endogenous β-cell). It is also contemplated that the population of pancreatic β-cells may also contain non-insulin producing cells (i.e. cells of β-cell like phenotype with the exception they do not produce or secrete insulin).

The term “exocrine cell” as used herein refers to a cell of an exocrine gland, i.e. a gland that discharges its secretion via a duct. In particular embodiments, an exocrine cells refers to a pancreatic exocrine cell, which is a pancreatic cell that produces enzymes that are secreted into the small intestine. These enzymes help digest food as it passes through the gastrointestinal tract. Pancratic exocrine cells are also known as islets of Langerhans, that secrete two hormones, insulin and glucagon. A pancreatic exocrine cell can be one of several cell types: alpha-2 cells (which produce the hormone glucagon); or β-cells (which manufacture the hormone insulin); and alpha-1 cells (which produce the regulatory agent somatostatin). Non-insulin producing exocrine cells as used herein refers to alpha-2 cells or alpha-1 cells. Note, the term pancreatic exocrine cells encompasses “pancreatic endocrine cells” which refer to a pancreatic cell that produces hormones (e.g., insulin (produced from β-cells) and glucagon (produced by alpha-2 cells) that are secreted into the bloodstream.

The term “pancreas” refers to a glandular organ that secretes digestive enzymes and hormones. In humans, the pancreas is a yellowish organ about 7 in. (17.8 cm) long and 1.5 in. (3.8 cm) wide. It lies beneath the stomach and is connected to the small intestine intestine, muscular hoselike portion of the gastrointestinal tract extending from the lower end of the stomach (pylorus) to the anal opening. Most of the pancreatic tissue consists of grapelike clusters of cells that produce a clear fluid (pancreatic juice) that flows into the duodenum through a common duct along with bile from the liver. Pancreatic juice contains three digestive enzymes: tryptase, amylase, and lipase, that, along with intestinal enzymes, complete the digestion of proteins, carbohydrates, and fats, respectively. Scattered among the enzyme-producing cells of the pancreas are small groups of endocrine cells, called the islets of Langerhans, that secrete two hormones, insulin and glucagon. The pancreatic islets contain several types of cells: alpha-2 cells, which produce the hormone glucagon; beta cells, which manufacture the hormone insulin; and alpha-1 cells, which produce the regulatory agent somatostatin. These hormones are secreted directly into the bloodstream, and together, they regulate the level of glucose in the blood. Insulin lowers the blood sugar level and increases the amount of glycogen (stored carbohydrate) in the liver; glucagon has the opposite action. Failure of the insulin-secreting cells to function properly results in diabetes or diabetes mellitus.

In some embodiments, the primary iPS cell is differentiated into a cell of endoderm origin (e.g. exocrine cells, pancreatic exocrine cells, acinar pancreatic cells, liver cells and gall bladder cells), which can be used to be reprogrammed to generate a secondary iPS (2iPS) cell. In general, a cell of endoderm origin be cultured under standard conditions of temperature, pH, and other environmental conditions, e.g., as adherent cells in tissue culture plates at 37° C. in an atmosphere containing 5-10% CO₂. The cells and/or the culture medium are appropriately modified to achieve reprogramming to a secondary iPS (2iPS) cell as described herein. In certain embodiments, cells of endoderm origin (e.g. exocrine cells, pancreatic exocrine cells, acinar pancreatic cells, liver cells and gall bladder cells) can be cultured on or in the presence of a material that mimics one or more features of the extracellular matrix or comprises one or more extracellular matrix or basement membrane components. In some embodiments Matrigel™ is used. Other materials include proteins or mixtures thereof such as gelatin, collagen, fibronectin, etc. In certain embodiments of the invention, cells of endoderm origin (e.g. exocrine cells, pancreatic exocrine cells, acinar pancreatic cells, liver cells and gall bladder cells) can be cultured in the presence of a feeder layer of cells. Such cells may, for example, be of murine or human origin. They can also be irradiated, chemically inactivated by treatment with a chemical inactivator such as mitomycin c, or otherwise treated to inhibit their proliferation if desired. In other embodiments cells of endoderm origin (e.g. exocrine cells, pancreatic exocrine cells, acinar pancreatic cells, liver cells and gall bladder cells) are cultured without feeder cells.

The term “endoderm cell” as used herein refers to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of respiratory and digestive tracts and the liver and pancreas.

A marker of a pancreatic β-cell is well known in the art, and include the expression of markers, but are not limited to, insulin (INS), NKX6 transcription factor related, locus 1 (NKX6.1), Nkx2.2, NeuroD, Glut2, glucokinase (GCK), prohormone convertase (PC1/3) and/or connecting peptide (C-peptide). A pancreatic β-cell may include the lack of expression of, but are not limited to, Amylase (Amy), Glucagon, somatostatin/pancreatic polypeptide (SomPP), Ptf1a, the duct marker Ck19 (also known as Krt19), and mesenchymal markers Nestin and Vimentin.

In another embodiment, a somatic first cell is a liver cell, e.g., a hepatic cell which can be reprogrammed into a primary iPS cell, which is differentiated along liver lineages to a second somatic cell. Methods to direct the differentiation of iPS cells along liver and hepatic lineages are well know by persons of ordinary skill in the art. In some embodiments, the second somatic cell is a liver cell, e.g., a hepatic cell. In some embodiments, the second somatic cell, e.g., liver cell, e.g., a hepatic cell is reprogrammed to a secondary iPS (2iPS) cell. In some embodiments, such a secondary iPS (2iPS) cell can spontaneously differentiate into liver cells, e.g., a hepatic cell.

Thus any cell type of a first somatic cell can be used to be reprogrammed to a primary iPS cell, which is then differentiated along a specific lineage to produce a second somatic cell, which is subsequently reprogrammed to a secondary iPSC (2iPSC), where the 2iPS cell has high efficiency of differentiating along a lineage to produce a somatic cell of the same cell type as the second somatic cell.

Thus, in some embodiments, a 2iPS cell has a high efficiency to differentiated into a cell of the same cell type as the second somatic cell, e.g., a somatic cells of a rare differentiation cell type fate such as but not limited to a ventricular cardiomyocyte, a pancreatic β-cell or a hepatic cell. In some embodiments, a 2iPS cell has a statistically significant increase in the efficiency to differentiate into a cell of the same cell type as the second somatic cell, e.g., a somatic cell of a rare differentiation cell type fate. In some embodiments, a 2iPS cell has an increased efficiency to differentiate into a somatic cells of a rare differentiation cell type fate by at least about 20%, or at least about 30%, or at least about 40%, or at least about 50%, or at least about 60%, or at least about 70%, or at least about 80%, or at least about 90%, or at least about 100%, or at least about 2-fold, or at least about 3-fold, or at least about 4-fold, or at least about 5-fold, or at least about 6-fold, or at least about 7-fold, or at least about 8-fold, or at least about 10-fold, as compared to the efficiency of other pluripotent stem cells, e.g., ES cells or primary iPS cells to differentiate into the cell type of the same rare differentiation cell type fate.

Thus, in some embodiments, the present invention relates to a method to produce a high yield of a somatic cells of a rare differentiation cell type fate, where the somatic cells of a rare differentiation cell type fate is differentiated from a 2iPS derived from a second somatic cell type which has a rare differentiation cell type fate, e.g., but is not limited to, a ventricular cardiomyocyte, a pancreatic β-cell or a hepatic cell. In some embodiments, the high yield is a statistically significant increase in the yield of somatic cells of a rare differentiation cell type fate differentiated from 2iPS cells as compared to such cells differentiated from other pluripotent stem cells, e.g., ES cells or primary iPS cells. In some embodiments, the increase in yield is at least about 20%, or at least about 30%, or at least about 40%, or at least about 50%, or at least about 60%, or at least about 70%, or at least about 80%, or at least about 90%, or at least about 100%, or at least about 2-fold, or at least about 3-fold, or at least about 4-fold, or at least about 5-fold, or at least about 6-fold, or at least about 7-fold, or at least about 8-fold, or at least about 10-fold increase in yield of somatic cells of a rare differentiation cell type fate differentiated from 2iPS cells as compared to such cells differentiated from other pluripotent stem cells, e.g., ES cells or primary iPS cells.

Sources of Human Somatic Cells for Reprogramming into Primary iPS Cells

One aspect of the present invention relates to the generation of ViPS cells, which are secondary iPS cells generated from reprogramming a cardiac cell which has been differentiated from a primary iPS cell. Stated another way, a somatic cardiac fibroblast cell is reprogrammed to a primary iPS cell, which is then differentiated into a second somatic cardiac cell, where the second somatic cell can be reprogrammed into a ventricular myocyte secondary iPS cell or ViPS cell.

In some embodiments, the cardiac fibroblast cells, e.g., mammalian cardiac cells, or mammalian cardiac fibroblasts can be obtained from the heart of any mammalian subject, e.g., a human subject, for example, embryonic tissue such as fetal or pre-fetal tissue, or adult cardiac tissue).

iPS cells (secondary or primary iPSCs) can be maintained in culture in a pluripotent state by routine passage until it is desired that they be differentiated into cardiac lineages.

In some embodiments, the ViPS cells can reprogrammed from cardiac somatic cells differentiated from primary iPS cells.

In some embodiments, a somatic cell can be reprogrammed into a iPS cell or 2iPS cell by any method known in the art can be used, for example virally-induced or chemically induced generation of iPS cells are described in Mauritz et al., Circulation. 2008; 118:507-517, and disclosed in International Application WO2008/088882, EP1970446, US2009/0047263, US2009/0068742, and 2009/0227032, which are incorporated herein in their entirety by reference.

iPS cells can also be generated using other methods commonly known in the art, such as, including but not limited to uses of non-viral methods, polycistronic vectors, mRNA species, miRNA, and proteins, including International Patent Applications WO2010/019569, WO2009/149233, WO2009/093022, WO2010/022194, WO2009/101084, WO2008/038148, WO2010/059806, WO2010/057614, WO2010/056831, WO2010/050626, WO2010/033906, WO2009/126250, WO2009/143421, WO2009/140655, WO2009/133971, WO2009/101407, WO2009/091659, WO2009/086425, WO2009/079007, WO2009/058413, WO2009/032456, WO2009/032194, WO2008/103462, JP4411362, EP2128245, and U.S. Patent Applications US2004/0072343, US2009/0253203, US2010/0112693, US2010/07542, US2009/0246875, US2009/0203141, US2010/00625343, US2009/0269763, US2010/059806 which are incorporated herein in their entirety by reference.

In some embodiments, iPS cells can be generated from synthetic modified RNAs (referred to as MOD-RNA), for example, as described in U.S. Provisional Application 61/387,220, filed Sep. 28, 2010, and U.S. Provisional Application 61/325,003, filed: Apr. 16, 2010, both of which are incorporated herein in their entirety by reference.

In some embodiments, the quality of a population of iPS cells for generating ventricular cardiomyocytes and then ViPS cell can be assessed, for example, using methods as disclosed in WO 2010/033906, which is incorporated herein in its entirety. For example, a population of primary iPS cells can be selected for high efficiency to differentiate along cardiogenic lineages, and for example into ventricular cardiomyocytes according to the methods as disclosed in WO 2010/033906, and such a suitable iPS cell line can be selected to differentiate into cardiac cells which can be subsequently reprogrammed to generate secondary iPS cells or ViPS according to the methods as disclosed herein.

In another embodiment, the iPS cell or 2iPS cell can be reprogrammed from cardiac cells, e.g., cardiac fibroblasts or from ventricular cardiomyocytes obtained from a subject, e.g., a mammalian subject including a human subject. In some embodiments, iPS cells are derived from reprogramming somatic cells of cardiac tissue, e.g., cardiac muscle tissue (e.g. cardiomyocytes) or cardiac vascular tissue. A mixture of cells from a suitable source of cardiac tissue is harvested from a mammalian donor by methods known in the art. The heart tissue is dissociated and cells plated in culture. Cardiac fibroblasts will adhere to the surface of the culture dish, and the supernatant comprising non-cardiac fibroblasts can be discarded to allow collecting the cardiac fibroblasts to reprogram to primary iPS cells or 2iPSCs.

The inventors demonstrate that the ViPS can be also be induced to differentiate and/or mature along different lineages (e.g. cardiomyocyte lineages, endothelial lineages and smooth muscle lineages) spontaneously and form beating cardiomyocytes after about 6 days in vitro. In some embodiments, culturing ViPS in conditioned media comprising addition of factors to induce differentiation is contemplated, by such methods that are commonly known in the art. Such conditions may also be referred to as cardiac differentiation conditions. For instance, any growth factors or differentiation-inducing factors can be added to the medium, as well as a supporting structure (such as a substrate on a solid surface) to induce differentiation. Differentiation may be initiated by allowing the stem cells to form aggregates, or similar structures, for example, aggregates can result from overgrowth of a stem cell culture, or by culturing the stem cells in culture vessels having a substrate with low adhesion properties.

In one embodiment of the invention, ViPS form embryoid bodies and can grow in suspension culture. About 90% or more of the ViPS embryoid bodies (EB) can spontaneously differentiate into cardiomyocytes after around about 5 days to around about 4 weeks. Viable differentiating cells from in vitro cultures of ViPS are selected that generate cardiac beating sheets or similar structures and are selected for phenotypic features using methods that substantially maintain the cell to cell contacts in the cell aggregate of beating EB or beating sheet.

### Three-Dimensional Human Vascularized Cardiac Tissue.

One aspect of the present invention relates to the use of ViPS cells to generate functional three-dimensional ventricular cardiomyocyte cardiac tissue. In one embodiment, ViPS cells can be differentiate into ventricular cardiomyocytes and each layer of cells stacked on another layer of cells to form a functional ventricular cardiomyocyte tissue. In some embodiments, the ViPS cells are implanted into a subject, where they spontaneously differentiate into functional ventricular cardiomyocytes and ventricular cardiac muscle, as well as other cell types, including smooth muscle progenitors, epithelial progenitors and cardiomyocyte progenitors, which further differentiate into smooth muscle tissue, vascular tissue and cardiac muscle respectively.

In some embodiments, the ViPS cells can be contacted with agents to increased differentiation into different cardiac tissue types. For example, cardiotrophic factors or growth factor to promote survival and/or growth of cardiac cells. Cardiotrophic factors are well known in the art and include but are not limited to cardiotrophic agents, creatine, carnitine, taurine, cardiotropic factors as disclosed in U.S. Patent Application Serial No. 2003/0022367 which is incorporated herein by reference, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway. Other cardiac enhancing peptides include cellular differentiation agents, such as cytokines and growth factors, as disclosed herein. Examples of various cell differentiation agents are disclosed in U.S. Patent Application Serial No. 2003/0022367 which is incorporated herein by reference, or Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001.

In some embodiments, the ViPS cells can be contacted with agents or proteins in order to enhance their ability to be involved in tissue regeneration, or to deliver a therapeutic protein to a site of administration. For example, one can contact the ViPS cells with one or more growth factors of various types, cardiotropic factors such as atrial natriuretic factor, cripto, and cardiac transcription regulation factors, such as GATA-4, Nkx2.5, and Mef2-C. Other examples of cytokines and growth factors include, but are not limited to, cardiotrophic agents, creatine, carnitine, taurine, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway.

In some embodiments, the ViPS cells are contacted with VEGF to increase vascularization or cardiovascular cells. In some embodiments, ViPS cells can be contacted with one or more synthetic modified RNAs to express a particular protein in the ViPS cells, for example VEGF, other cardiotrophic factors and agents as disclosed in U.S. provisional patent application 61/471,166 filed Apr. 3, 2011, which is incorporated herein in its entirety by reference.

In some embodiments, a population of ViPS cells, e.g., human ViPS can be implanted into a subject, and proportion of ViPS within the population will differentiate along cardiomyocyte lineages, e.g., ventricular cardiomyocytes, a proportion of ViPS cells, e.g., human ViPS cells will differentiate along cardiac endothelial lineages and a proportion of ViPS cells, e.g., human ViPS cells will differentiate along cardiac smooth muscle lineages. Accordingly, one aspect of the present invention relates to the use of ViPS cells, e.g., human ViPS to spontaneously differentiate into ventricular cardiomyocytes to generate a functional three-dimensional ventricular cardiac muscle, with out the need to direct the differentiation of the ViPS cells along specific cardiac lineages using other manipulations (e.g. addition of growth factors or angiogenic agents or other agents).

In some embodiments, a population of ViPS cells, e.g., human ViPS implanted into a subject can undergo coordinated differentiation into cardiomyocytes, endothelial cells and smooth muscle cells, such that ViPS derived cardiac cells form a functional cardiac tissue.

In some embodiments, when a population of ViPS cells, e.g., human ViPS cells is implanted into a subject, some ViPS cells will self-replicate (e.g. proliferate or renew), and some will spontaneously differentiate along cardiomyocyte lineages, e.g., into ventricular cardiomyocytes. In some embodiments, ViPS cells, e.g., human ViPS cells can be directed to differentiate along other cardiac lineages, e.g., endothelial lineages to produce an endothelial progenitor, or differentiate along a smooth muscle lineage to produce a smooth muscle progenitor. In such instances, these human cardiomyocyte progenitors, human endothelial progenitors and human smooth muscle progenitors can self-replicate (e.g. proliferate) before terminally differentiating into cardiomyocytes, endothelial cells and smooth muscle cells respectively.

In some embodiments, the ViPS cells, e.g., human ViPS cells, or the differentiated progeny, e.g., ventricular cardiomyocytes are implanted into a subject, for example a human, for example for therapeutic purposes, or an animal subject. In some embodiment, ViPS, e.g., human ViPS cellscan be implanted into any suitable location in a subject, for example but not limited to heart, pericardium, epicardium, on the surface of the heart, in a pericardial space, and the like.

In some embodiments, a population of ViPS, e.g., human ViPS cellscan be encapsulated in a bioreactor bag, which can be implanted into a subject at a suitable location, for example on the surface of the heart, subcutaneously or the like. Examples of a bioreactor include, for example, a sleeve comprising ViPS cells, e.g., human ViPS cells, or ViPS-derived ventricular cardiomyocytes can be used as a patch to place on the surface of the heart, wherein the sleeve comprises a biocompatible structure encircling a length of the tissue, or circumferentially surrounding or enclosing the tissue. As used herein, “a length of the tissue” refers to at least 50% of the total length of the tissue, or at least 80%, 90% or even greater than the length of the tissue. Where multiple organized tissues are contained within a sleeve, the sleeve will encompass “a length” of at least one such organized tissue, and possibly also of two, three, four or more plural organized tissues. The sleeved organized tissue according to the invention also includes a sleeved tissue wherein the tissue is substantially encapsulated or surrounded (i.e., encircled along a length, where the length of encirclement is at least 50% of the length of the tissue, or 80%, 90%, or fully encapsulated) by the sleeve. Examples of bioreactors are disclosed in U.S. Patent Applications 2005/0260178; 2004/0043010, 2003/0235561 and 2003/0180264 which are incorporated herein in their entirety by reference.

The ViPS, e.g., human ViPS cells as disclosed herein can spontaneously differentiate at a high efficiency into ventricular cardiomyocytes. In some embodiments, ViPS cells can spontaneously differentiate at a lower efficiency than ventricular cardiomyocytes into other cardiac cell types, e.g., any cell type of the heart, including but not limited to, epicardial cells, endothelial cells, and smooth muscle cells, including pace maker cells, Purkinje cells, and cells which make up the vascular coronary tree, the population of ViPS cells can be implanted into a subject can form functional human vascularized cardiac tissue.

In some embodiments, the ViPS cells can be used to differentiate into cardiac cells to produce functional 3D cardiac tissue has substantially the same properties of normal functional endogenous human myocardium, including but not limited to, substantially the same contracting force, contraction frequency, contraction stamina, and vascularization as endogenous human myocardium.

In some embodiments, the ViPS cells, e.g., human ViPS cells, or ViPS-derived ventricular cardiomyocytes can be implanted into a subject in the absence of a matrix or scaffold. In some embodiments, ViPS cells, e.g., human ViPS cells, or ViPS-derived ventricular cardiomyocytes can be implanted into a subject in the presence of a three-dimensional matrix or scaffold, as defined herein. In some embodiments, the scaffold can be a patterned polymer scaffold as disclosed in International Patent Application PCT/US2009/060224 or WO2008/045506, which are incorporated herein in their entirety by reference. Alternatively, one of ordinary skill in the art can use any scaffold, for example biocompatible or bioresorbable scaffolds, including those disclosed in US Patent Applications: 2008/019229 and International Application WO/2003/050266, which are incorporated herein in their entirety by reference.

In some embodiments, ViPS cells, e.g., human ViPS cells, or ViPS-derived ventricular cardiomyocytes can be implanted into a subject in the absence of growth factors. Alternatively, in some embodiments, ViPS cells, e.g., human ViPS cells, or ViPS-derived ventricular cardiomyocytes can be implanted into a subject in the presence of at least one or a combination of growth factors, such as cardiotrophic factors as disclosed herein, or at least one or a combination of angiogenic factors.

### Use of ViPS Cells or Ventricular Cardiomyocytes for Assays In Vivo

In some embodiments where ViPS cells, e.g., human ViPS cells, or ViPS-derived ventricular cardiomyocytes are implanted into an animal subject, the animal can be used as an in vivo humanized model of vascular disease. For example, an animal model which comprises ViPS cells, e.g., human ViPS cells, or ViPS-derived ventricular cardiomyocytes, can be used to screen for agents which affect any one, or a combination of viability, functionality, contractibility, differentiation of the human cardiac tissue.

Accordingly, one embodiment relates to the use of ViPS cells, e.g., human ViPS cells, or ViPS-derived ventricular cardiomyocytes as an assay, for example to assess drug toxicity (e.g. cardiotoxicity) on human heart tissue in vivo (e.g. to identify agents which increase apoptosis, decrease viability, modulate (e.g. increase or decrease by a statistically significantly amount) contractibility and/or conductivity of heart tissue). In some embodiments, the drugs and/or compounds can be existing drugs or compounds, and in other embodiments, the drugs or compounds can be new or modified drugs and compounds.

In another embodiment, ViPS cells, e.g., human ViPS cells, or ViPS-derived ventricular cardiomyocytes can be used as an assay for example to identify agents which increase and decrease coronary blood flow to human vascularized heart tissue in vivo. For example, ViPS cells, e.g., human ViPS cells, or ViPS-derived ventricular cardiomyocytes could be given atherosclerosis, for example by implanting the ViPS cells, e.g., human ViPS cells, or ViPS-derived ventricular cardiomyocytes into a LDR −/− mouse and feeding the mouse a high fat diet.

Another aspect of the invention relates use of ViPS cells, e.g., human ViPS cells, or ViPS-derived ventricular cardiomyocytes as disclosed herein to screen for agents, for example molecules and genes involved in biological events. In such an embodiment, the biological event is an event that affects the spontaneous differentiation of a ViPS, e.g., human ViPS cells into ventricular cardiomyocytes. Also, the ViPS, e.g., human ViPS cells can be used in assays to identify any agent which promotes the differentiation, proliferation, survival, regeneration, maintenance of the ViPS-derived ventricular cardiomyocytes, and/or inhibition or down-regulation of differentiation of ViPS cells into ViPS-derived ventricular cardiomyocytes.

In another embodiment, ViPS cells, e.g., human ViPS cells, or ViPS-derived ventricular cardiomyocytes can be used to assess the effect of genetic variation (e.g. ethnicity, human mutations or gene variants or polymorphism) on cardiac function. For example, the effect of different environmental factors, such as, for example, obesity, high fat diet, lack of exercise, can be assessed ventricular cardiomyocytes differentiated from ViPS cells from different ethnicities and genetic backgrounds. In an alternative embodiment, the effect (e.g. efficacy and/or safety profile) of different therapeutic agents and cardiac drugs can be assessed in human ventricular cardiomyocytes differentiated from ViPS cells from different ethnicities and genetic backgrounds. Accordingly, in some embodiments, ViPS cells can be used to study a cardiovascular disease as well as identify which therapeutic agents are effective in the treatment of cardiovascular diseases and disorders from different ethnicities and genetic backgrounds, particularly where the ViPS cells are generated from reprogramming of secondary cardiac cells obtained from primary iPS cells, where the first cardiac cell was obtained from a subject to a genetic variant and/or a genetically modified primary iPS cell.

In another embodiment, ViPS cells, e.g., human ViPS cells, or ViPS-derived ventricular cardiomyocytes as disclosed herein can be used as an assay for example to identify other cells which can be implanted in combination with the ViPS cells, e.g., human ViPS cells, or ViPS-derived ventricular cardiomyocytes, for example, addition of committed ventricular progenitors (CVP) as disclosed in International Patent Application PCT/US2009/060224, which is incorporated herein in its entirety by reference.

In another embodiment, ViPS cells, e.g., human ViPS cells, or ViPS-derived ventricular cardiomyocytes can be used in an assay for studying the efficiency of differentiation of ViPS or iPS cells into ventricular cardiomyocytes as compared to the efficiency of differentiating into multiple other cardiac lineages, for example but not limited to, human cardiac, human smooth muscle and human endothelial cell lineages. In some embodiments as disclosed herein, ViPS cells, e.g., human ViPS cells can be genetically engineered to comprise markers operatively linked to promoters that are expressed in one or more of the lineages being studied, e.g., expressed in ventricular cardiomyocytes.

In some embodiments, ViPS cells, e.g., human ViPS cells, can be used in an assay for studying the differentiation pathway of ViPS cells into subpopulations of human cardiomyocytes. In some embodiments, the ViPS cells, e.g., human ViPS cells can be genetically engineered to comprise markers operatively linked to promoters that drive gene transcription in specific cardiomyocyte subpopulations, for example but not limited to atrial, ventricular, outflow tract and conduction systems. In other embodiments, ViPS cells, e.g., human ViPS cells can be used in an assay for studying the role of cardiac mesenchyme on cardiovascular stem cells.

In alternative embodiments, ViPS cells, e.g., human ViPS cells, or ViPS-derived ventricular cardiomyocytes can comprise a mutation and/or polymorphism that relates to the disease phenotype, and in other embodiments, the ViPS, e.g., human ViPS cells has been genetically engineered to carry a mutation and/or polymorphism.

Any suitable animal can be used for implanting a population of ViPS cells, e.g., human ViPS cells, or ViPS-derived ventricular cardiomyocytes, for example, rodents (such as mice, rats), monkeys, pigs and the like. In some embodiments, the subject animal is a transgenic or knockout animal, such as transgenic mice or knockout mice. In some embodiments, the subject animal is a humanized mouse, such as the SCID mouse.

In some embodiments, ViPS cells, e.g., human ViPS cells, or ViPS-derived ventricular cardiomyocytes as disclosed herein can be used to identify an agent for the effect of the agent has on the ventricular cardiomyocyte as assessed by monitoring output parameters, such as expression of markers, cell viability, differentiation characteristics, multipotential capacity of ViPS cells and the like.

In some embodiments, ViPS cells, e.g., human ViPS cells, or ViPS-derived ventricular cardiomyocytes can be implanted into an immunodeficient animal (such as nude mice, such as SCID mice, or animals rendered immunodeficient chemically or by irradiation) for an in vivo model of the differentiation of ViPS cells along ventricular cardiomyocyte lineage.

The effect of an agent administered to an animal comprising ViPS cells, e.g., human ViPS cells, or ViPS-derived ventricular cardiomyocytes can be assessed by the degree of cardiac recuperation that ensues from inflicting injury to the human vascular tissue. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N2 on the anterior wall of the left ventricle for approximately 20 min (Chiu et al., Ann. Thorac. Surg. 60:12, 1995). Infarction can be induced by ligating the left main coronary artery (Li et al., J. Clin. Invest. 100:1991, 1997). Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, and rate of pressure decay.

In other embodiments, ViPS cells, e.g., human ViPS cells, or ViPS-derived ventricular cardiomyocytes as disclosed herein can be used as models for studying the development of a normal heart or, alternatively, the ViPS, e.g., human ViPS cells are generated from a subject with a congenital heart abnormality, the ViPS cells, e.g., human ViPS cells, or ViPS-derived ventricular cardiomyocytes can be used to study the differentiation of ventricular cardiomyocytes and the formation of a heart with a congenital heart abnormality or dysfunction. In some embodiments the ViPS, e.g., human ViPS cells carry a mutation and/or polymorphism, and in other embodiments, ViPS, e.g., human ViPS cells have been genetically engineered to carry a mutation and/or polymorphism.

In some embodiments, ViPS cells, e.g., human ViPS cells, or ViPS-derived ventricular cardiomyocytes can be used in assays to screen agents, selected from a group of a chemical, small molecule, chemical entity, nucleic acid sequences, an action; nucleic acid analogues or protein or polypeptide or analogue of fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof.

In some embodiment, at least one agent is administered to a subject comprising ViPS cells, e.g., human ViPS cells, or ViPS-derived ventricular cardiomyocytes or added to a culture media culturing ViPS cells, e.g., human ViPS cells, or ViPS-derived ventricular cardiomyocytes as disclosed herein by any suitable means known to one of ordinary skill in the art. In some embodiments, if the ViPS cells, e.g., human ViPS cells, or ViPS-derived ventricular cardiomyocytes are present in a subject, e.g., an animal model, administration occurs more than once, for example at multiple different time points. In some embodiments, the administration of an agent to a subject, e.g., an animal model comprising ViPS cells, e.g., human ViPS cells, or ViPS-derived ventricular cardiomyocytes is continuous, for example via means of an infusion pump or catheter or the like, or via a slow-release formulation of the agent. In some embodiments, the agent is administered to a subject locally to the site of the ViPS cells, e.g., human ViPS cells, or ViPS-derived ventricular cardiomyocytes in the subject.

In some embodiments, an agent is administered to a subject comprising ViPS cells, e.g., human ViPS cells, or ViPS-derived ventricular cardiomyocytes via any or a combination of the following administration methods; systemic administration, intravenous, transdermal, intrasynovial, intramuscular, oral administration, parenteral administration, intraarterial administration, intrathecal administration, intraventricular administration, intraparenchymal, intracranial, intracisternal, intrastriatal, and intranigral administration, and intracoronary administration.

In some embodiments, the agents are conveniently administered to a subject comprising ViPS cells, e.g., human ViPS cells, or ViPS-derived ventricular cardiomyocytes in a pharmacological applicable carrier, such as solution, or readily soluble form. The agents may be added in a pump (e.g. flow-through system), as a stream, intermittent or continuous, or alternatively, adding a bolus of the compound, singly or incrementally, to an otherwise static solution. In some embodiments, agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

In some embodiments, an agent may be applied to the media comprising ViPS cells, e.g., human ViPS cells, or ViPS-derived ventricular cardiomyocytes prior to the implantation into the subject, where the agent contacts the ViPS cells, e.g., human ViPS cells, or ViPS-derived ventricular cardiomyocytes and induces its effects. Alternatively, the agent may be intracellular within the ViPS, e.g., human ViPS, as a result of introduction of the nucleic acid sequence into the cell and its transcription resulting in the production of the nucleic acid and/or protein agent within the cell.

The term “agent” refers to any chemical, entity or moiety, including without limitation synthetic and naturally-occurring non-proteinaceous entities. In certain embodiments the compound of interest is a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

In some embodiments, the agent is an agent of interest including known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidate agents also include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as agents are pharmacologically active drugs, genetically active molecules, etc. Compounds of interest include, for example, chemotherapeutic agents, hormones or hormone antagonists, growth factors or recombinant growth factors and fragments and variants thereof. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

Agents are screened for effect on ViPS cells, e.g., human ViPS cells, or ViPS-derived ventricular cardiomyocytes by administering at least one agent to a subject comprising ViPS cells, e.g., human ViPS cells, or ViPS-derived ventricular cardiomyocytes. The change in parameters in response to the agent is measured, and the result evaluated by comparison to reference agents or absence of agent or other controls one of ordinary skill in the art would use.

Parameters of ViPS cells, e.g., human ViPS cells, or ViPS-derived ventricular cardiomyocytes can be quantifiable components of humanized vascular heart tissue, particularly parameters that can be accurately measured, desirably in real time and/or in vivo when the ViPS cells, e.g., human ViPS cells, or ViPS-derived ventricular cardiomyocytes are present in live animal model.

In some embodiments, ViPS cells, e.g., human ViPS cells, or ViPS-derived ventricular cardiomyocytes can be genetically modified to express markers, e.g. bioluminescence markers, such as luciferase and the like and other bioluminescent markers commonly known in the art for real-time imaging of the function, and/or growth or differentiation of the ViPS cells, e.g., human ViPS cells, or ViPS-derived ventricular cardiomyocytes in real time. The is advantageous as it allows the continuous and/or time-point analysis of the effect of an agent on ViPS cells, e.g., human ViPS cells, or ViPS-derived ventricular cardiomyocytes over a period of time, as well as allows one to compare the effect of multiple different agents (administered to the subject at different timepoints).

In some embodiments, quantifiable parameters of ViPS-derived ventricular cardiomyocytes can include contractile force, contractibility, cardiomyocyte atrophy, altered contraction, frequency of contraction, contraction duration, contraction stamina, vascularization of ventricular cardiac muscle tissue produced from a plurality of ViPS-derived ventricular cardiomyocytes. In some embodiments, quantifiable parameters differentiation, survival and regeneration of the ViPS-derived ventricular cardiomyocytes.

While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

A plurality of assays comprising ViPS cells, e.g., human ViPS cells, or ViPS-derived ventricular cardiomyocytes may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Optionally, the ViPS cells, e.g., human ViPS cells, or ViPS-derived ventricular cardiomyocytes used in the screen can be manipulated to express desired gene products. Gene therapy can be used to either modify a cell to replace a gene product or add or knockdown a gene product. In some embodiments the genetic engineering is done to facilitate regeneration of tissue, to treat disease, or to improve survival of the cells following implantation into a subject. In some embodiments, the ViPS cells, e.g., human ViPS cells, or ViPS-derived ventricular cardiomyocytes is genetically modified to express a stress survival factor so the ViPS-derived ventricular cardiomyocytes can survive contraction, e.g. such as expression of truncated Creb312 as disclosed in U.S. provisional application 61/145,208, filed on Jan. 16, 2009, which is incorporated herein by reference. In some embodiments, a population of ViPS cells, e.g., human ViPS cells, or ViPS-derived ventricular cardiomyocytes is genetically modified to express at least one gene which prevent rejection. Techniques for transfecting cells are known in the art.

A skilled artisan could envision a multitude of genes which would convey beneficial properties to the transfected ViPS cells, e.g., human ViPS cells, or ViPS-derived ventricular cardiomyocytes to be used in implantation (discussed in more detail below). The added gene may ultimately remain in the recipient ViPS cells, e.g., human ViPS cells, or ViPS-derived ventricular cardiomyocytes and all its progeny, or may only remain transiently, depending on the embodiment. For example, genes encoding angiogenic factors could be transfected into ViPS cells, e.g., human ViPS cells, or ViPS-derived ventricular cardiomyocytes then go to form ventricular cardiac muscle as disclosed herein. Such genes would be useful for inducing collateral blood vessel formation as the ventricular cardiac muscle tissue is regenerated. It some situations, it may be desirable to transfect the ViPS cells, e.g., human ViPS cells, or ViPS-derived ventricular cardiomyocytes with more than one gene.

In some instances, it is desirable to have the gene product secreted from the ViPS cells, e.g., human ViPS cells, or ViPS-derived ventricular cardiomyocytes. In such cases, the gene product preferably contains a secretory signal sequence that facilitates secretion of the protein. For example, if the desired gene product is an angiogenic protein, a skilled artisan could either select an angiogenic protein with a native signal sequence, e.g. VEGF, or can modify the gene product to contain such a sequence using routine genetic manipulation (See Nabel et al., 1993).

The desired gene can be transfected into ViPS cells, e.g., human ViPS cells, or ViPS-derived ventricular cardiomyocytes using a variety of techniques. Preferably, the gene is transfected into the ViPS cells, e.g., human ViPS cells, or ViPS-derived ventricular cardiomyocytes using an expression vector. Suitable expression vectors include plasmid vectors (such as those available from Stratagene, Madison Wis.), viral vectors (such as replication defective retroviral vectors, herpes virus, adenovirus, adeno-virus associated virus, and lentivirus), and non-viral vectors (such as liposomes or receptor ligands).

The desired gene is usually operably linked to its own promoter or to a foreign promoter which, in either case, mediates transcription of the gene product. Promoters are chosen based on their ability to drive expression in restricted or in general tissue types, for example in mesenchymal cells, or on the level of expression they promote, or how they respond to added chemicals, drugs or hormones. Other genetic regulatory sequences that alter expression of a gene may be co-transfected. In some embodiments, the host cell DNA may provide the promoter and/or additional regulatory sequences. Other elements that can enhance expression can also be included such as an enhancer or a system that results in high levels of expression.

Methods of targeting genes in mammalian cells are well known to those of skill in the art (U.S. Pat. Nos. 5,830,698; 5,789,215; 5,721,367 and 5,612,205). By “targeting genes” it is meant that the entire or a portion of a gene residing in the chromosome of a cell is replaced by a heterologous nucleotide fragment. The fragment may contain primarily the targeted gene sequence with specific mutations to the gene or may contain a second gene. The second gene may be operably linked to a promoter or may be dependent for transcription on a promoter contained within the genome of the cell. In a preferred embodiment, the second gene confers resistance to a compound that is toxic to cells lacking the gene. Such genes are typically referred to as antibiotic-resistance genes. Cells containing the gene may then be selected for by culturing the cells in the presence of the toxic compound.

Methods of gene targeting in mammals are commonly used to generate “knockout” or modified ES cells, which can be applied to human or marine ES cells (U.S. Pat. Nos. 5,616,491; 5,614,396, which are incorporated herein in their entirety by reference). These techniques take advantage of the ability of embryonic stem cells to promote homologous recombination, an event that is rare in differentiated mammalian cells. Recent advances in human embryonic stem cell culture may provide a needed component to applying the technology to human systems (Thomson; 1998). Furthermore, the methods of the present invention can be used to isolate and enrich for stem cells or progenitor cells that are capable of homologous recombination and, therefore, subject to gene targeting technology. Indeed, the ability to isolate and grow somatic stem cells and progenitor cells has been viewed as impeding progress in human gene targeting (Yanez & Porter, 1998).

### Therapeutic Uses of ViPS Cells, e.g., Human ViPS Cells, or ViPS-Derived Ventricular Cardiomyocytes

Another embodiment relates to the therapeutic use of ViPS cells, e.g., human ViPS cells, or ViPS-derived ventricular cardiomyocytes, for example, in one embodiment the invention provides methods for the treatment cardiovascular disorders and/or congenital heart disease in a subject comprising transplanting into subjects ViPS cells, e.g., human ViPS cells, or ViPS-derived ventricular cardiomyocytes.

In one embodiment, ViPS cells, e.g., human ViPS cells, or ViPS-derived ventricular cardiomyocytes can be used for the production of a pharmaceutical composition, for the use in transplantation into subjects in need of cardiac tissue transplantation, for example but not limited to subjects with congenital and/or acquired heart disease and/or subjects with vascular diseases. In one embodiment, ViPS cells, e.g., human ViPS cells, or ViPS-derived ventricular cardiomyocytes can be genetically modified. In another aspect, the subject can have or be at risk of heart disease and/or vascular disease. In some embodiments, ViPS cells, e.g., human ViPS cells, or ViPS-derived ventricular cardiomyocytes can be autologous and/or allogenic. In some embodiments, ViPS cells, e.g., human ViPS cells, or ViPS-derived ventricular cardiomyocytes for transplanted are immunogenetically matched to the transplant receipt (e.g. blood type and HLA matched). In some embodiments, for autologous transplantation, a cardiac somatic cell, e.g., a cardiac fibroblast or ventricular cardiomyocyte is obtained from a subject (who will receive the ViPS) and the first cardiac cell is reprogrammed to a primary iPS cell. The primary iPS cell can be directed to differentiate along cardiac lineages to differentiate into second cardiac cell, e.g., a cardiac fibroblast or ventricular cardiomyocyte, which can subsequently be reprogrammed to a secondary iPS-VM cell or ViPS cell. In some embodiments, a population of ViPS cells (or iPS cells derived from ventricular cardiomyocytes), which initially originated from a first cardiac cell which was reprogrammed to a primary iPS cell, are transplanted into the same subject from which the first cardiac somatic cell was obtained.

In some embodiments, a subject in which ViPS cells, e.g., human ViPS cells, or ViPS-derived ventricular cardiomyocytes are implanted for therapeutic purposes is a mammal, such as a human.

In one embodiment of the invention relates to a method of treating a circulatory disorder comprising administering an effective amount of a composition comprising ViPS cells, e.g., human ViPS cells, or ViPS-derived ventricular cardiomyocytes as disclosed herein to a subject with a circulatory disorder. In a further embodiment, the invention provides a method for treating myocardial infarction, comprising administering a composition comprising human vascularized cardiac tissue to a subject having a myocardial infarction in an effective amount sufficient to produce human vascularized cardiac tissue in the heart of the individual.

The invention further provides for a method of treating an injured tissue in an individual comprising: (a) determining a site of tissue injury in the individual; and (b) administering ViPS cells, e.g., human ViPS cells, or ViPS-derived ventricular cardiomyocytes of the invention in a composition into and around the site of tissue injury, e.g., cardiac infarction. In one embodiment, the ViPS cells, e.g., human ViPS cells, or ViPS-derived ventricular cardiomyocytes are derived from an autologous source. In a further embodiment, the tissue injury is a myocardial infarction, cardiomyopathy or congenital heart disease

In one embodiment of the above methods, the subject is a human and the composition is human ViPS cells, or human ViPS-derived ventricular cardiomyocytes. In alternative embodiments, ViPS cells, e.g., human ViPS cells, or ViPS-derived ventricular cardiomyocytes can be used to treat circulatory disorder is selected from the group consisting of cardiomyopathy, myocardial infarction, and congenital heart disease. In some embodiments, the circulatory disorder is a myocardial infarction. The invention provides that ViPS cells, e.g., human ViPS cells, or ViPS-derived ventricular cardiomyocytes can be used to treat myocardial infarction by reducing the size of the myocardial infarct or replacing the infarct scar. It is also contemplated that the ViPS cells, e.g., human ViPS cells, or ViPS-derived ventricular cardiomyocytes can be used to treat myocardial infarction by reducing the size of the scar resulting from the myocardial infarct. The invention contemplates administration of ViPS cells, e.g., human ViPS cells, or ViPS-derived ventricular cardiomyocytes directly to heart tissue of a subject, or is administered systemically by any means known by one of ordinary skill in the art.

In some embodiments, ViPS cells, e.g., human ViPS cells, or ViPS-derived ventricular cardiomyocytes can, with or without a three-dimensional matrix, be used as a “patch” or band aid at site of cardiac injury or cardiac dysfunction. In some embodiments, the ViPS cells, e.g., human ViPS cells, or ViPS-derived ventricular cardiomyocyte serves as a “patch” or band aid at site of myocardial infarction. In some embodiments, ViPS cells, e.g., human ViPS cells, or ViPS-derived ventricular cardiomyocytes can be applied as a patch to the heart. In some embodiment, the patch can be attached by any means, such as by a fixative, glue, sutures and staples and the like. In some embodiments, ViPS cells, e.g., human ViPS cells, or ViPS-derived ventricular cardiomyocytes are administered to a site of myocardial infarction in a gel composition.

In certain embodiments, ViPS cells, e.g., human ViPS cells, or ViPS-derived ventricular cardiomyocytes can be attached to at least one tissue e.g., heart or ventricle at the time of implantation, using techniques known in the art. Exemplary attachment means include suturing, stapling, for example, with surgical staples, glue or adhesive, such as surgical glue, biochemical interactions such as with the extracellular matrix, photo-activated glue, fibrin glue, acrylate-based adhesives, and the like.

The present invention is also directed to a method of treating circulatory damage in the heart or peripheral vasculature which occurs as a consequence of genetic defect, physical injury, environmental insult or damage from a stroke, heart attack or cardiovascular disease (most often due to ischemia) in a subject, the method comprising administering (including implanting or transplanting) ViPS cells, e.g., human ViPS cells, or ViPS-derived ventricular cardiomyocytes to a subject. Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

In some embodiments, ViPS cells, e.g., human ViPS cells, or ViPS-derived ventricular cardiomyocytes is useful for the treatment of congenital cardiovascular abnormalities. In some embodiments, the recipient subject is an adult, and in some embodiments, the recipient subject is a neonate or child such as a human baby or human child. In some embodiments, ViPS cells, e.g., human ViPS cells, or ViPS-derived ventricular cardiomyocytes can be implanted into a neonate, which has the advantage that it will grow into appropriate cardiovascular structures, e.g. valves, myocardium etc with the normal development of the neonate thorough childhood and into adulthood. This is advantage as current methodologies using cardiac tissue implants or grafts for treatment of neonates and young children with congenital cardiovascular defects do not grow and develop as the child develops, and thus such children or subjects with severe congenital defects need multiple surgeries to repair the heart, and often do not survive beyond 2 years of age.

In some embodiments, the effects of administration of ViPS cells, e.g., human ViPS cells, or ViPS-derived ventricular cardiomyocytes would be demonstrated by, but not limited to, one of the following clinical measures: increased heart ejection fraction, decreased rate of heart failure, decreased infarct size, decreased associated morbidity (pulmonary edema, renal failure, arrhythmias) improved exercise tolerance or other quality of life measures, and decreased mortality. The effects of the administration of human vascularized cardiac tissue can be evident over the course of days to weeks after the procedure. However, beneficial effects may be observed as early as several hours after the procedure, and may persist for several years.

In some embodiments, the ViPS cells, e.g., human ViPS cells, or ViPS-derived ventricular cardiomyocytes can be implanted as a patch for the diaphragm. This can be useful for treatment of diseases or disorders where diaphragm function is impaired, as well as diseases such as Duchene Muscular dystrophy. In some embodiments, the ViPS cells, e.g., human ViPS cells, or ViPS-derived ventricular cardiomyocytes can be genetically modified to increase survival post implantation, for example express the mechanical stretch survival factor, such as Creb312 as disclosed herein, and/or express the normal dystrophin gene.

In some embodiments, the human vascularized cardiac tissue are administered to a subject in need thereof in a manner that permits the ViPS cells, e.g., human ViPS cells, or ViPS-derived ventricular cardiomyocytes to graft and/or migrate to the intended tissue site and reconstitute or regenerate the functionally deficient area. Special devices are available that are adapted for administering the ViPS cells, e.g., human ViPS cells, or ViPS-derived ventricular cardiomyocytes capable of reconstituting cardiac function directly to the chambers of the heart, the pericardium, or the interior of the cardiac muscle at the desired location. ViPS cells, e.g., human ViPS cells, or ViPS-derived ventricular cardiomyocytes may be administered to a recipient heart by intracoronary injection, e.g. into the coronary circulation. The human vascularized cardiac tissue may also be administered by intramuscular injection into the wall of the heart.

In some embodiments, ViPS cells, e.g., human ViPS cells, or ViPS-derived ventricular cardiomyocytes for treatment can comprise at least one type of additional cells, such as CVP cells as disclosed herein.

In some embodiments, ViPS cells, e.g., human ViPS cells, or ViPS-derived ventricular cardiomyocytes can be implanted into a subject in need thereof in the presence of growth factors, and/or angiogenic factors. In some embodiments, ViPS cells, e.g., human ViPS cells, or ViPS-derived ventricular cardiomyocytes are genetically modified to express the growth factors and/or angiogenic factors as disclosed herein, under the control of constitutive, or tissue-specific or inducible promoters. Exemplary cytokines include angiogenin, vascular endothelial growth factor (VEGF, including, but not limited to VEGF-165), interleukins, fibroblast growth factors, for example, but not limited to, FGF-1 and FGF-2, hepatocyte growth factor, (HGF), transforming growth factor beta (TGF-.beta.), endothelins (such as ET-1, ET-2, and ET-3), insulin-like growth factor (IGF-1), angiopoietins (such as Ang-1, Ang-2, Ang-3/4), angiopoietin-like proteins (such as ANGPTL1, ANGPTL-2, ANGPTL-3, and ANGPTL-4), platelet-derived growth factor (PDGF), including, but not limited to PDGF-AA, PDGF-BB and PDGF-AB, epidermal growth factor (EGF), endothelial cell growth factor (ECGF), including ECGS, platelet-derived endothelial cell growth factor (PD-ECGF), placenta growth factor (PLGF), and the like. The skilled artisan will understand that the choice of chemokines and cytokine fragments to be expressed ViPS cells, e.g., human ViPS cells, or ViPS-derived ventricular cardiomyocytes, or administered with the ViPS cells, e.g., human ViPS cells, or ViPS-derived ventricular cardiomyocytes will, in part, on the target tissue or organ to be implanted with the human vascularized cardiac tissue.

The compositions comprising ViPS cells, e.g., human ViPS cells, or ViPS-derived ventricular cardiomyocytes can have a variety of uses in clinical therapy, research, development, and commercial purposes. For therapeutic purposes, for example, ViPS cells, e.g., human ViPS cells, or ViPS-derived ventricular cardiomyocytes may be administered to enhance tissue maintenance or repair of cardiac muscle for any perceived need, such as an inborn error in metabolic function, the effect of a disease condition, or the result of significant trauma. The human vascularized cardiac tissue that are administered to the subject not only help restore function to damaged or otherwise unhealthy tissues, but also facilitate remodeling of the damaged tissues.

To determine the suitability of ViPS cells, e.g., human ViPS cells, or ViPS-derived ventricular cardiomyocytes for therapeutic administration, the human vascularized cardiac tissue can first be tested in a suitable animal model commonly known by persons of ordinary skill in the art. At one level, ViPS cells, e.g., human ViPS cells, or ViPS-derived ventricular cardiomyocytes is assessed for its ability to survive and maintain function in vivo, or the spontaneous differentiation of ViPS cells into ventricular cardiomyocytes in vivo.

ViPS cells, e.g., human ViPS cells, or ViPS-derived ventricular cardiomyocytes can be administered in any physiologically acceptable excipient. ViPS cells, e.g., human ViPS cells, or ViPS-derived ventricular cardiomyocytes may be introduced by injection, catheter, or the like. In some embodiments, t ViPS cells, e.g., human ViPS cells, or ViPS-derived ventricular cardiomyocytes can be cryopreserved (e.g. frozen) at liquid nitrogen temperatures and stored for long periods of time, being capable of use on thawing. If frozen, the cells will usually be stored in a 10% DMSO, 50% FCS, 40% RPMI 1640 medium. Once thawed, the human vascularized cardiac tissue can be cultured ex vivo in the presence of growth factors and/or feeder layers for an appropriate period of time prior to implanting into a subject in need thereof.

ViPS cells, e.g., human ViPS cells, or ViPS-derived ventricular cardiomyocytes as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition comprising ViPS cells, e.g., human ViPS cells, or ViPS-derived ventricular cardiomyocytes may also comprise or be accompanied with one or more other ingredients that facilitate the engraftment or functional mobilization of the cells. Suitable ingredients include matrix proteins that support or promote adhesion of the cells, or complementary cell types, especially endothelial cells. In another embodiment, the composition may comprise resorbable or biodegradable matrix scaffolds.

As discussed herein, in some embodiments, ViPS cells, e.g., human ViPS cells, or ViPS-derived ventricular cardiomyocytes can be generated from primary iPS cells which are genetically altered in order to introduce genes useful in the ViPS cells, e.g., human ViPS cells, or ViPS-derived ventricular cardiomyocytes, e.g. repair of a genetic defect in an individual, selectable marker, etc., or genes useful in selection against cells which are ViPS cells, e.g., human ViPS cells, or ViPS-derived ventricular cardiomyocytes. As discussed, ViPS cells, e.g., human ViPS cells, or ViPS-derived ventricular cardiomyocytes can be generated from iPS cells or ViPS cells which are genetically modified to enhance survival, control proliferation, and the like. ViPS cells, e.g., human ViPS cells, or ViPS-derived ventricular cardiomyocytes may be genetically altering by transfection or transduction with a suitable vector, homologous recombination, or other appropriate technique, so that they express a gene of interest. In one embodiment, the ViPS cells, e.g., human ViPS cells, or ViPS-derived ventricular cardiomyocytes can be transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. In some embodiments, the ViPS cells, e.g., human ViPS cells, or ViPS-derived ventricular cardiomyocytes can be genetically altered using vector containing supernatants over a 8-16 h period, and then exchanged into growth medium for 1-2 days. Genetically altered ViPS cells, e.g., human ViPS cells, or ViPS-derived ventricular cardiomyocytes can be selected using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (i.e. prevent rejection).

In an alternative embodiment, ViPS cells, e.g., human ViPS cells, or ViPS-derived ventricular cardiomyocytes can be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are ViPS cells, e.g., human ViPS cells, or ViPS-derived ventricular cardiomyocytes that are genetically altered to express one or more growth factors of various types, cardiotropic factors such as atrial natriuretic factor, cripto, and angiogenic factors such as VEGF.

Many vectors useful for transferring exogenous genes into target ViPS cells, e.g., human ViPS cells, or ViPS-derived ventricular cardiomyocytes s are available. The vectors may be episomal, e.g. plasmids, virus derived vectors such as cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PAl2 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g. AKR env, are capable of infecting most mammalian cell types, except murine cells. In some embodiments, the vectors may include genes that must later be removed, e.g. using a recombinase system such as Cre/Lox, or the cells that express them destroyed, e.g. by including genes that allow selective toxicity such as herpesvirus TK, Bcl-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

In one aspect of the present invention, the ViPS cells, e.g., human ViPS cells, or ViPS-derived ventricular cardiomyocytes can be administered systemically or to a target anatomical site. In some embodiments, ViPS cells, e.g., human ViPS cells, or ViPS-derived ventricular cardiomyocytes can be grafted into or nearby a subject's heart, for example, or may be administered systemically, such as, but not limited to, intra-arterial or intravenous administration.

In alternative embodiments, ViPS cells, e.g., human ViPS cells, or ViPS-derived ventricular cardiomyocytes as disclosed herein can be administered in various ways as would be appropriate to implant in the cardiovascular system, including but not limited to parenteral, including intravenous and intraarterial administration, intrathecal administration, intraventricular administration, intraparenchymal, intracranial, intracisternal, intrastriatal, and intranigral administration. Optionally, ViPS cells, e.g., human ViPS cells, or ViPS-derived ventricular cardiomyocytes are administered in conjunction with an immunosuppressive agent.

In some embodiments, ViPS cells, e.g., human ViPS cells, or ViPS-derived ventricular cardiomyocytes as disclosed herein can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. Implantation of ViPS cells, e.g., human ViPS cells, or ViPS-derived ventricular cardiomyocytes into a recipient subject can take place but is not limited to the following locations: clinic, clinical office, emergency department, hospital ward, intensive care unit, operating room, catheterization suites, and radiologic suites.

In other embodiments, at least a portion of the ViPS cells, e.g., human ViPS cells, or ViPS-derived ventricular cardiomyocytes originating from a subject can be stored for later implantation/infusion. In some embodiments, ViPS cells, e.g., human ViPS cells, or ViPS-derived ventricular cardiomyocytes can be divided into more than one aliquot or unit such that a population ViPS cells, e.g., human ViPS, or ViPS-derived ventricular cardiomyocytes is retained for later application while part is applied immediately to the subject. Moderate to long-term storage of all or a proportion of the population of ViPS cells, e.g., human ViPS cells, or ViPS-derived ventricular cardiomyocytes can be stored in a cell bank, and is also within the scope of this invention, as disclosed in U.S. Patent Application Serial No. 20030054331 and Patent Application No. WO03024215, and is incorporated by reference in their entireties. At the end of processing, the human vascularized cardiac tissue as disclosed herein may be loaded into a delivery device, such as a syringe, for placement into the recipient subject by any means known to one of ordinary skill in the art.

Pharmaceutical Composition:

In some embodiments, the present invention relates to a composition comprising ViPS, e.g., human ViPS, and/or ViPS-derived ventricular cardiomyocytes as disclosed herein. In some embodiments, all compositions can further comprise a differentiation agent. Differentiation agents for use in the present invention are well known to those of ordinary skill in the art. Examples of such agents include, but are not limited to, cardiotrophic agents, creatine, carnitine, taurine, cardiotropic factors as disclosed in U.S. Patent Application Serial No. 2003/0022367 which is incorporated herein by reference, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway. The pharmaceutical compositions may further comprise a pharmaceutically acceptable carrier.

ViPS cells, e.g., human ViPS cells, or ViPS-derived ventricular cardiomyocytes present in a composition can be administered to a subject alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. In some embodiments, a ViPS cells, e.g., human ViPS cells, or ViPS-derived ventricular cardiomyocyte composition can also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, compositions comprising ViPS cells, e.g., human ViPS cells, or ViPS-derived ventricular cardiomyocytes can be combined with a gene encoding pro-angiogenic and/or cardiomyogenic growth factor(s) which allow the ViPS cells, e.g., human ViPS cells, or ViPS-derived ventricular cardiomyocytes to act as their own source of growth factor during cardiac repair or regeneration. Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid’ adeno-associated virus.

In some embodiments, a composition comprising ViPS cells, e.g., human ViPS cells, or ViPS-derived ventricular cardiomyocytes can be implanted into a subject along with a carrier material bearing gene delivery vehicle capable of releasing and/or presenting genes to the cells over time such that transduction can continue or be initiated.

In some embodiments, particularly when a composition comprising ViPS cells, e.g., human ViPS cells, or ViPS-derived ventricular cardiomyocytes are administered to a subject other than the subject from whom the first cardiac cell were obtained, one or more immunosuppressive agents may be administered to the subject receiving the composition comprising ViPS cells, e.g., human ViPS cells, or ViPS-derived ventricular cardiomyocytes to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 20020182211. In one embodiment, a immunosuppressive agent is cyclosporine A. Other examples include mycophenolate mofetil, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiovascular stem cells of the invention.

In certain embodiments of the invention, a composition comprising ViPS cells, e.g., human ViPS cells, or ViPS-derived ventricular cardiomyocytes can be administered to a patient with one or more cellular differentiation agents, such as cytokines and growth factors, as disclosed herein. Examples of various cell differentiation agents are disclosed in U.S. Patent Application Serial No. 2003/0022367 which is incorporated herein by reference, or Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001. Other examples of cytokines and growth factors include, but are not limited to, cardiotrophic agents, creatine, carnitine, taurine, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, and products of the BMP or cripto pathway.

In some embodiments, a composition comprising ViPS cells, e.g., human ViPS cells, or ViPS-derived ventricular cardiomyocytes can comprise an effective number of cells, optionally, in combination with a pharmaceutically acceptable carrier, additive or excipient. In certain aspects of the present invention, a composition comprising ViPS cells, e.g., human ViPS cells, or ViPS-derived ventricular cardiomyocytes can be are administered to the subject in need of a transplant in sterile saline. In other aspects of the present invention, a composition comprising ViPS cells, e.g., human ViPS cells, or ViPS-derived ventricular cardiomyocytes can be administered in Hanks Balanced Salt Solution (HBSS) or Isolyte S, pH 7.4. Other approaches may also be used, including the use of serum free cellular media. In one embodiment, a composition comprising ViPS cells, e.g., human ViPS cells, or ViPS-derived ventricular cardiomyocytes can be administered in plasma or fetal bovine serum, and DMSO. Systemic administration of a composition comprising ViPS cells, e.g., human ViPS cells, or ViPS-derived ventricular cardiomyocytes to the subject may be preferred in certain indications, whereas direct administration at the site of or in proximity to the diseased and/or damaged tissue may be preferred in other indications.

In some embodiments, a composition comprising ViPS cells, e.g., human ViPS cells, or ViPS-derived ventricular cardiomyocytes can optionally be packaged in a suitable container, in the presence of an suitable media. In some embodiments, the package further comprises written instructions for a desired purpose, such as methods of implantation into a subject (and optionally methods of storage and/or methods of thawing if cryopreserved) of the composition comprising ViPS cells, e.g., human ViPS cells, or ViPS-derived ventricular cardiomyocytes for the improvement of an abnormality of cardiac function or the treatment of cardiovascular disease. In some embodiments, treatment can refer to a reduction of a cardiovascular disorder. In alternative embodiments, treatment is prophylactic treatment to prevent the development of a cardiac disorder where the subject is at risk of developing a cardiovascular disorder as disclosed herein.

In one embodiment, a composition comprising ViPS cells, e.g., human ViPS cells, or ViPS-derived ventricular cardiomyocytes can be administered with a differentiation agent. In one embodiment, a composition comprising ViPS cells, e.g., human ViPS cells, or ViPS-derived ventricular cardiomyocytes can be combined with the differentiation agent and administered (e.g. implanted) concurrently to the subject. In another embodiment, a composition comprising ViPS cells, e.g., human ViPS cells, or ViPS-derived ventricular cardiomyocytes are administered separately to the subject from the differentiation agent. Optionally, if a composition comprising ViPS cells, e.g., human ViPS cells, or ViPS-derived ventricular cardiomyocytes are administered separately from the differentiation agent, there is a temporal separation in the administration of the composition comprising ViPS cells, e.g., human ViPS cells, or ViPS-derived ventricular cardiomyocytes and the differentiation agent. The temporal separation may range from about less than a minute in time, to about hours or days in time. The determination of the optimal timing and order of administration is readily and routinely determined by one of ordinary skill in the art.

In some embodiments of the present invention may be defined in any of the following numbered paragraphs:

1. A method of generating an induced pluripotent stem (iPS) cell comprising: (i) reprogramming a first somatic cell by inducing the expression of at least one reprogramming factor to produce a first induced pluripotent stem cell; (ii) differentiating the first induced pluripotent stem cell along a specific cell linage to generate a second somatic cell of a target cell type fate; (iii) reprogramming the second somatic cell by inducing the expression of at least one reprogramming factor to produce a second induced pluripotent stem cell, wherein the second induced pluripotent stem cell has a high efficiency to differentiate into a somatic cell of a target cell type fate.
2. A method of generating an induced pluripotent stem (iPS) cell which can differentiate into a ventricular cardiomyocyte comprising: (i) reprogramming a first somatic cell by inducing the expression of at least one reprogramming factor to produce a first induced pluripotent stem cell; (ii) differentiating the first induced pluripotent stem cell along a cardiogenic lineage to generate a second cardiac cell; (iii) reprogramming the second cardiac cell by inducing the expression of at least one reprogramming factor to produce a second induced pluripotent stem cell, wherein the second induced pluripotent stem cell can differentiate into a ventricular cardiomyocyte.
3. The method of paragraph 1 or 2, wherein the first somatic cell is a fibroblast.
4. The method of paragraph 1 or 2, wherein the first somatic cell is a skin fibroblast.

5. The method of paragraph 1 or 2, wherein the first somatic cell is a cardiac fibroblast.

6. The method of paragraphs 1 or 2, wherein the first somatic cell is a human somatic cell.
7. The method of paragraphs 1, 2 or 5, wherein the first somatic cell is a human cardiac fibroblast.
8. The method of paragraph 1 or 2, wherein the first somatic cell is a cardiomyocyte.
9. The method of paragraph 2, wherein the second cardiac cell is a cardiomyocyte.
10. The method of paragraph 8 or 9, wherein the cardiomyocyte is a ventricular cardiomyocyte.
11. The method of paragraph 8 or 9, wherein the cardiomyocyte is a human cardiomyocyte.
12. The method of paragraphs 1 or 2, wherein inducing the expression of reprogramming factor in the first cardiac cell comprises introducing genes encoding at least one reprogramming factor operatively linked to an inducible promoter.
13. The method of paragraph 1, wherein inducing the expression of reprogramming factor in the second somatic cell comprises contacting the second cardiac cell with an agent to induce expression of the genes encoding at least one reprogramming factor operatively linked to an inducible promoter.
14. The method of paragraph 2, wherein inducing the expression of reprogramming factor in the second cardiac cell comprises contacting the second cardiac cell with an agent to induce expression of the genes encoding at least one reprogramming factor operatively linked to an inducible promoter.
15. The method of any of paragraphs 1 or 2, wherein the reprogramming factor comprises at least one gene product.
16. The method of any of paragraphs 1 or 2, wherein the reprogramming factor comprises at least one protein.
17. The method of any of paragraphs 1 or 2, wherein the reprogramming factor is encoded by a gene contained in a recombinant vector introduced into the first somatic cell.
18. The method of paragraph 16, wherein the gene encoding the reprogramming factor is operatively linked to an inducible promoter.
19. The method of any of paragraphs 1 to 18, wherein the reprogramming factor comprises the gene product of an Oct family gene.
20. The method of any of paragraphs 1 to 18, wherein the reprogramming factor comprises the gene product of a Klf family gene.
21. The method of any of paragraphs 1 to 18, wherein the reprogramming factor comprises the gene product of a Myc family gene.
22. The method of any of paragraphs 1 to 18, wherein the reprogramming factor comprises the gene product of a Sox family gene.
23. The method of any of paragraphs 1 to 18, wherein the reprogramming factor comprises one or more gene products selected from the group consisting of: Oct family gene, Klf family gene, Sox family gene, Myc family gene
24. The method of any of paragraphs 1 to 18, wherein the reprogramming factor comprises one or more gene products selected from the group consisting of: Oct3/4, Klf4, Sox2, c-Myc.
25. The method of any of paragraphs 1 to 24, wherein the reprogramming factor further comprises one or more gene products of a Sal4 gene.
26. The method of any of paragraphs 1 to 25, wherein the reprogramming factor comprises one or more gene products selected from the group consisting of: Oct3/4, Klf4, Sox2, c-Myc, Nanog, Lin28.
27. The method of any of paragraphs 1 to 26, wherein differentiating the first induced pluripotent stem cell along cardiogenic lineages comprising contacting the first induced pluripotent stem cell with a cardiomyocyte differentiating agent selected from the group consisting of: cardiotrophic agents, creatine, carnitine, taurine, activin A, activin B, insulin-like growth factors, bone morphogenic proteins, fibroblast growth factors, platelet-derived growth factor natriuretic factors, insulin, leukemia inhibitory factor (LIF), epidermal growth factor (EGF) and TGFα.
28. The method of any of paragraphs 1 to 27, wherein the second cardiac cell expresses at least one of any of the following cardiac markers; Mlc2v, cTnT.
29. The method of any of paragraphs 1 to 28, wherein the second iPS cell expresses at least one of any of the following markers at a significantly higher level than ES cells or a primary iPS cell; Nkx2.5, Gata4, Isl1, myocardin and Tbx20.
30. The method of any of paragraphs 1 to 29, wherein the first somatic cell which is reprogrammed to a first iPS cell is a postnatal somatic cell, or an adult somatic cell.
31. The method of any of paragraphs 1 to 30, wherein the second somatic cell which is reprogrammed is a postnatal somatic cell, or an adult somatic cell.
32. The method of any of paragraphs 1 to 31, wherein the second somatic cell which is reprogrammed is not an embryonic somatic cell.
33. The method of paragraph 1, wherein the second somatic cell which is reprogrammed to a second iPS cell not an embryonic somatic cell.
34. The method of paragraph 1, wherein the second somatic cell of a target cell type fate is a cell of a rare differentiation cell type fate.
35. The method of paragraph 34, wherein the rare differentiation cell type fate is selected from the group consisting of: a pancreatic β-cell, a hepatic cell, a ventricular cardiomyocyte cell, lung cell, and other somatic cells of a rare differentiation cell type fate.
36. The method of paragraph 31, wherein the first induced pluripotent stem cell is differentiated along a specific cell linage to generate a second somatic cell selected from the group of: a pancreatic β-cell, a hepatic cell, a ventricular cardiomyocyte cell, lung cell, and other somatic cells of a rare differentiation cell type fate.
37. A ventricular cardiomyocyte obtained by reprogramming a secondary induced pluripotent stem (iPS) cell by the method of paragraph 2.
38. The ventricular cardiomyocyte of paragraph 37, wherein the ventricular cardiomyocyte is a mammalian ventricular cardiomyocyte.
39. The ventricular cardiomyocyte of paragraph 37, wherein the ventricular cardiomyocyte is a human ventricular cardiomyocyte.
40. A method for stem cell therapy comprising; (i) collecting at least a first somatic cell from a subject; (ii) reprogramming the first somatic cell from the subject into a first induced pluripotent (iPS) cell by inducing the expression of at least one reprogramming factor; (iii) differentiating the first induced pluripotent (iPS) cell into a second somatic cell; (iv) reprogramming the second somatic cell into a second induced pluripotent (iPS) cell by inducing the expression of at least one reprogramming factor; and (iv) transplanting the second induced pluripotent (iPS) cell, or a differentiated progeny somatic cell derived from the second induced pluripotent (iPS) cell into the subject.
41. The method of paragraph 40, wherein the second induced pluripotent stem cell has a high efficiency of differentiating into the same cell type of the second somatic cell as compared to a cell differentiated from the first induced pluripotent stem cell.
42. The method of any of paragraphs 40 to 41, wherein the first somatic cell is a human cell.
43. The method of any of paragraphs 40 to 41, wherein the first somatic cell is a fibroblast somatic cell.
44. The method of any of paragraphs 40 to 43, wherein the first somatic cell is a cardiac fibroblast somatic cell.
45. The method of any of paragraphs 40 to 44, wherein the first somatic cell is a human cardiac fibroblast somatic cell.
46. The method of any of paragraphs 40 to 45, wherein the second somatic cell is selected from the group consisting of: a pancreatic β-cell, a ventricular cardiomyocyte, a lung cell, a hepatic cell.
47. The method of any of paragraphs 40 to 46, wherein the second somatic cell is a ventricular cardiomyocyte.
48. The method of any of paragraphs 40 to 47, wherein the second somatic cell is an adult somatic cell or a postnatal somatic cell.
49. The method of any of paragraphs 40 to 48, wherein the second somatic cell is not an embryonic somatic cell.
50. The method of any of paragraphs 40 to 49, wherein the subject is a human subject.
51. The method of any of paragraphs 40 to 50, wherein the differentiated progeny of a second induced pluripotent stem cell is a ventricular cardiomyocyte.
52. A method for stem cell therapy comprising; (i) reprogramming the first somatic cell obtained from the subject into a first induced pluripotent (iPS) cell by inducing the expression of at least one reprogramming factor; (ii) differentiating the first induced pluripotent (iPS) cell into a second cardiac cell; (iii) reprogramming the second cardiac cell into a second induced pluripotent (iPS) cell by inducing the expression of at least one reprogramming factor; and (iii) transplanting the second induced pluripotent (iPS) cell, or their differentiated progeny into the subject.
53. The method of paragraph 52, wherein the second pluripotent stem cell has a high efficiency of differentiating into the same cell type of the second somatic cell as compared to a cell differentiated from the first induced pluripotent stem cell.
54. The method of paragraph 52, wherein the first somatic cell is a human cell.
55. The method of paragraph 52, wherein the first somatic cell is selected from a fibroblast somatic cell, or a cardiac fibroblast somatic cell.
56. The method of paragraph 52, wherein the second cardiac cell is a ventricular cardiomyocyte.
57. The method of paragraph 52, wherein the second cardiac cell is an adult somatic cell or a postnatal somatic cell.
58. The method of paragraph 52, wherein the second cardiac cell is not an embryonic somatic cell.
59. The method of paragraph 52, wherein the subject is a human subject.
60. The method of paragraph 52, wherein the differentiated progeny of a second induced pluripotent stem cell is a ventricular cardiomyocyte.
61. Use of the method of paragraph 52 for treating a subject with a cardiovascular disease or disorder.
62. Use of a population of ventricular cardiomyocytes of paragraph 37 in an assay to evaluate the toxicity of an agent, wherein a population of ventricular cardiomyocytes are contacted with an agent compound and the contractile activity of a population of ventricular cardiomyocytes is measured.
63. The use of paragraph 61, wherein the contractile activity of a population of ventricular cardiomyocytes in the presence of the agent is compared to the contractile activity of a population of ventricular cardiomyocytes in the absence of the agent.
64. The use of paragraph 61, wherein a change in the contractile activity by a statistically significant amount in the presence of the agent as compared to the contractile activity in the absence of the agent identifies an agent that alters the contractile activity.
65. The use of paragraph 62, wherein a change in the contractile activity is an increase or decrease in at lease one contractile activity, and wherein a contractile activity is selected from the group consisting of: contractile force, contractile frequency, contractile duration and contractile stamina.

The following written description provides exemplary methodology and guidance for carrying out many of the varying aspects of the present invention.

Molecular Biology Techniques: Standard molecular biology techniques known in the art and not specifically described are generally followed as in Sambrook et al., Molecular Cloning: A Laboratory Manual, Cold Springs Harbor Laboratory, N.Y. (1989, 1992), and in Ausubel et al., Current Protocols in Molecular Biology, John Wiley and Sons, Baltimore, Md. (1989). Polymerase chain reaction (PCR) is carried out generally as in PCR Protocols: A Guide to Methods and Applications, Academic Press, San Diego, Calif. (1990). Reactions and manipulations involving other nucleic acid techniques, unless stated otherwise, are performed as generally described in Sambrook et al., Molecular Cloning: A Laboratory Manual, Cold Springs Harbor Laboratory Press, and methodology as set forth in U.S. Pat. Nos. 4,666,828; 4,683,202; 4,801,531; 5,192,659; and 5,272,057 and incorporated herein by reference. In situ PCR in combination with Flow Cytometry can be used for detection of cells containing specific DNA and mRNA sequences (see, for example, Testoni et al., Blood, 1996, 87:3822).

Immunoassays: Standard methods in immunology known in the art and not specifically described are generally followed as in Stites et al. (Eds.), Basic And Clinical Immunology, 8^{th} Ed., Appleton & Lange, Norwalk, Conn. (1994); and Mishell and Shigi (Eds.), Selected Methods in Cellular Immunology, W. H. Freeman and Co., New York (1980).

In general, immunoassays are employed to assess a specimen such as for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and RIA, can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al., Molecular Cloning: A Laboratory Manual, Cold Springs Harbor, N.Y., 1989. Numerous other references also may be relied on for these teachings.

Further elaboration of various methods that can be utilized for quantifying the presence of the desired marker include measuring the amount of a molecule that is present. A convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein (GFP) chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

Antibody Production: Antibodies may be monoclonal, polyclonal, or recombinant. Conveniently, the antibodies may be prepared against the immunogen or immunogenic portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Springs Harbor, N.Y. (1988) and Borrebaeck, Antibody Engineering—A Practical Guide by W. H. Freeman and Co. (1992). Antibody fragments may also be prepared from the antibodies and include Fab and F(ab′)2 by methods known to those skilled in the art. For producing polyclonal antibodies a host, such as a rabbit or goat, is immunized with the immunogen or immunogenic fragment, generally with an adjuvant and, if necessary, coupled to a carrier; antibodies to the immunogen are collected from the serum. Further, the polyclonal antibody can be absorbed such that it is monospecific. That is, the serum can be exposed to related immunogens so that cross-reactive antibodies are removed from the serum rendering it monospecific.

For producing monoclonal antibodies, an appropriate donor is hyperimmunized with the immunogen, generally a mouse, and splenic antibody-producing cells are isolated. These cells are fused to immortal cells, such as myeloma cells, to provide a fused cell hybrid that is immortal and secretes the required antibody. The cells are then cultured, and the monoclonal antibodies harvested from the culture media.

For producing recombinant antibodies, messenger RNA from antibody-producing B-lymphocytes of animals or hybridoma is reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker The antibody, or antibody fragment, is expressed using a suitable expression system. Antibody cDNA can also be obtained by screening pertinent expression libraries. The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982). The binding of antibodies to a solid support substrate is also well known in the art. (see for a general discussion Harlow & Lane, Antibodies: A Laboratory Manual, Cold Spring Harbor Laboratory Publications, New York, 1988 and Borrebaeck, Antibody Engineering—A Practical Guide, W. H. Freeman and Co., 1992). The detectable moieties contemplated with the present invention can include, but are not limited to, fluorescent, metallic, enzymatic and radioactive markers. Examples include biotin, gold, ferritin, alkaline phosphates, galactosidase, peroxidase, urease, fluorescein, rhodamine, tritium, 14C, iodination and green fluorescent protein.

Gene therapy and genetic engineering of cardiovascular stem cells and/or mesenchymal cells: Gene therapy as used herein refers to the transfer of genetic material (e.g., DNA or RNA) of interest into a host to treat or prevent a genetic or acquired disease or condition. The genetic material of interest encodes a product (e.g., a protein, polypeptide, and peptide, functional RNA, antisense, RNA, microRNA, siRNA, shRNA, PNA, pcPNA) whose in vivo production is desired. For example, the genetic material of interest encodes a hormone, receptor, enzyme polypeptide or peptide of therapeutic value. Alternatively, the genetic material of interest encodes a suicide gene. For a review see “Gene Therapy” in Advances in Pharmacology, Academic Press, San Diego, Calif., 1997.

With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & Isenberg eds, CRC Press 1989), Heart Development (Harvey & Rosenthal, Academic Press 1998).

The present invention is further illustrated by the following examples which in no way should be construed as being further limiting, The contents of all cited references, including literature references, issued patents, published patent applications, and co-pending patent applications, cited throughout this application are hereby expressly incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Materials and Methods

Mouse Lines and Constructs

The Mlc2v^{Cre/+}; Rosa26^{YFP/rtTA}; TgMef2c-AHF-dsRed compound transgenic animals were obtained by crossing Mlc2v^{Cre/+}; Rosa26^{rtTA/rtTA} males with Rosa26^{YFP/YFP}; TgMef2c-AHF-dsRed females. Mlc2v^{Cre/+} knock-in mouse line was generated in the lab as described (Chen, Kubalak et al. 1998). Rosa26^{YFP/YFP} conditional reporter mouse line (Srinivas, Watanabe et al. 2001) was purchased from the Jackson Laboratory. Rosa26^{rtTA/rtTA} mouse line was kindly provided by Dr. Konrad Hochedlinger (Hochedlinger, Yamada et al. 2005). TgMef2c-AHF-dsRed transgenic mouse line was generated in the lab as described (Domian, Chiravuri et al. 2009). The constructs used to produce lentivirus for iPS cell derivation were kindly provided by Dr. Konrad Hochedlinger (Stadtfeld, Maherali et al. 2008).

Production of Lentivirus

To produce infectious lentiviral particles, 293FT cells (purchased from Invitrogen) cultured on 10-cm dishes were transfected with the expression vectors together with the packaging plasmids pMD2G and pCMV-dR8.74 using Fugene HD (Roche). The viral particles for each reprogramming factor were produced individually. Virus-containing medium was harvested on 2 consecutive days starting 48 hours after transfection and used for infection without concentration.

Generation of the Primary iPS Cells from Cardiac Fibroblasts

To collect cardiac fibroblasts from neonatal pups, the hearts were digested with Trypsin (USB) at 4° C. overnight, followed by Collagenase type 2 (Worthington) at 37° C. for 30 minutes, and dissociated by triturating 10 times. The cardiac fibroblasts were enriched by differential centrifugation and plating. 20,000 cardiac fibroblasts were plated per well on a 6-well plate the day before infection. Virus containing medium was applied on the cells with polybrene (Sigma). Two infections were performed on the cells. Forty-eight hours after infection, the medium was changed to ES cell medium with 1 ug/ml Doxycycline (Sigma), which was withdrawn in 12 days. The putative iPS cells colonies were picked two days later after the withdrawal of Doxycycline.

Blastocyst Injection

Individual iPS/ES cells were microinjected into E3.5 blastocysts from C57B1/6 females and implanted into the uteri of pseudopregnant CD-1 foster mothers. To trace the TiPS cells used in the blastocyst injections to test their pluripotency, the conditional reporter allele Rosa26^{YFP} in the cells was activated by transient expression of an exogenous Cre in the cells before injection.

Generation of the Secondary iPS Cells from Ventricular Cardiomyocytes and Tail Tip Fibroblasts

The ventricular cardiomyocytes and tail tip fibroblasts from one neonatal chimeric pup were used for the generation of the secondary iPS cells. To collect ermeabilized, the heart was digested Trypsin (USB) at 4° C. overnight, followed by Collagenase type 2 (Worthington) at 37° C. for 30 minutes, and dissociated by triturating 10 times. The cardiomyocyte fraction was enriched by differential centrifugation and plating, after which the cardiomyocytes were plated directly on MEF feeder for the derivation of the secondary iPS cells. To collect tail tip fibroblasts, the tail tip was minced in Trypsin and cultured with DMEM with 10% FBS for 4 days. The attached tail tip fibroblasts were detached with Trypsin and plated on MEF feeder for the derivation of the secondary iPS cells. After the cardiomyocytes and tail tip fibroblasts were plated on MEF feeder, Doxycycine was added into the medium to re-induce the expression of the reprogramming transgenes. In two weeks, putative iPS cell colonies emerged. For the secondary iPS cells from ventricular myocytes, YFP+ colonies were picked for further study. For the secondary iPS cells from tail tip fibroblasts, colonies were picked randomly.

Generation of Mlc2v^{Cre/+}; Rosa26^{YFP/rtTA}; TgMef2c-AHF-dsRed compound transgenic ES cell lines

Timed matings were performed between Mlc2v^{Cre/+}; Rosa26^{rtTA/rtTA} males with Rosa26^{YFP/YFP}; TgMef2c-AHF-dsRed females. On day 3.5 PC, the females were sacrificed and the blastocysts flushed from the uterine horns with M2 medium (Sigma-Aldrich). The zona pellucida was removed with acidic Tyrode's Solution (Sigma-Aldrich) and the blastocysts were further washed three times with M2 media. The blastocysts were then plated onto mouse embryonic feeder cells (MEF) with ES cell derivation medium (DMEM with 15% KOSR, pen/strep, sodium pyruvate, nonessential amino acids, and leukemia inhibitory factor [LIF] [Chemicon, CA]).

In Vitro Differentiation of iPS/ES Cells Through Embryoid Body (EB) Formation

ES cells were cultured for at least two passages and then adapted for EB formation in adaptation medium (IMDM with 15% FBS, pen/strep, sodium pyruvate, nonessential amino acids and LIF) for two days. The adapted cells were digested to individual cells and re-suspended in differentiation medium (IMDM [Invitrogen] with 15% FBS, pen/strep, nonessential amino acids and ascorbic acid) at a concentration of 45,000 cells/ml. Hanging drops each containing about 540,000 cells were made with the cell suspension and cultured for two days, during which the cells in each hanging drop aggregated to form one EB. The EBs were cultured in differentiation medium until the day of analysis.

Real Time PCR

RNA from ES/iPS cells or EBs was extracted with Rneasy mini kit (Qiagen) and cDNA was synthesized with iScript kit (Bio-Rad). Real time PCR was performed using the I-cycler system with SYBR Green substrate (BioRad) for 40 cycles. Primer sequences are available on request.

Immunofluorescence Studies

Antibodies used in this study include antibody against the following proteins: SSEA1 (Hybridoma Bank at Iowa), Nanog (Cosmo Bio), Oct3/4 (Santa Cruz) and cardiac Troponin T. The cells were fixed with 4% PFA and blocked with PBS containing 10% FBS. Then the cells were incubated with blocking solution with the primary antibody. Subsequently, samples were incubated with the appropriate secondary antibodies conjugated with Alexa-Fluoro 594.

Intracellular FACS Analysis

EBs less than 7 days old were dissociated with Trypsin, and those more than 7 days with Trypsin followed by Collagenase A and B. The resultant individual cells were fixed with 2% PFA (Sigma), and blocked and permeabilized with PBS containing 10% FBS and 0.5% Tween 20. The cell were then incubated with the primary antibody overnight at 4° C., washed with the blocking solution, and incubated with the secondary antibody for 1 hour at room temperature. They were washed with PBS and analyzed with FACSCalibur (BD Biosciences).

### Example 1

Each of the chambers of the mammalian heart is composed of a specific population of cardiomyocytes that arise largely from the first and second heart fields (1). After a myocardial infarction, it is estimated that approximately a billion cardiomyocytes are lost (2); thus, one of the major hurdles for cardiac regenerative medicine will be generating sufficient numbers of cardiomyocytes necessary for effective cell replacement therapy (3). Ventricular myocytes, in particular, have distinct functional and electrophysiological properties that are optimized for efficient contractile function and electrical stability (4); therefore, it would be desirable to derive a homogenous population of ventricular myocytes since cardiomyocytes with varying functional and electrical properties in the wrong cardiac environment may be ineffective, counterproductive, or potentiate adverse cardiac arrhythmias (3, 5). A critical discovery has been the identification and purification of committed ventricular muscle cell progenitors (CVPs) of the second heart field lineage from embryos and mouse embryonic stem cells (3). CVPs have been used to engineer sheets of muscular thin films illustrating that a renewable source of ventricular myocyte precursors may be a potentially valuable resource for engineering cardiac patches that may be useful clinically (6).

Induced pluripotent stem (iPS) cells have been derived from a number of somatic cell types and are a potential source of autologous cardiomyocytes for cardiac repair (7, 8); however, the yield of cardiomyocytes from iPS cells or ES cells is generally low and can vary considerably between cell line to cell line (9, 10). Furthermore, the cardiomyocytes derived from iPS cells and ES cells display heterogeneous action potential properties with typically about 35-75% of cardiomyocytes demonstrating a ventricular phenotype (11, 12). Induced pluripotent stem cells retain a “memory” of their starting cell type that influences their molecular and differentiation properties (13, 14).

Here, the inventors have successfully derived iPS cells from heart progenitors and ventricular myocytes. Further, the vast majority (up to 99.5%) of cardiomyocytes derived from ViPS cells display a ventricular phenotype. DNA methylation analysis of ViPS cells and genetically-matched iPS cells from fibroblasts demonstrate distinct methylation patterns at cardiogenic loci suggesting that the mechanism of the ventriculogenic bias is due in part to residual methylation signatures of the starting ventricular myocyte. The ability to derive abundant numbers of pure ventricular myocytes addresses a critical requirement in the advancement of pluripotent stem cell models and therapeutics for cardiac disease (3, 15).

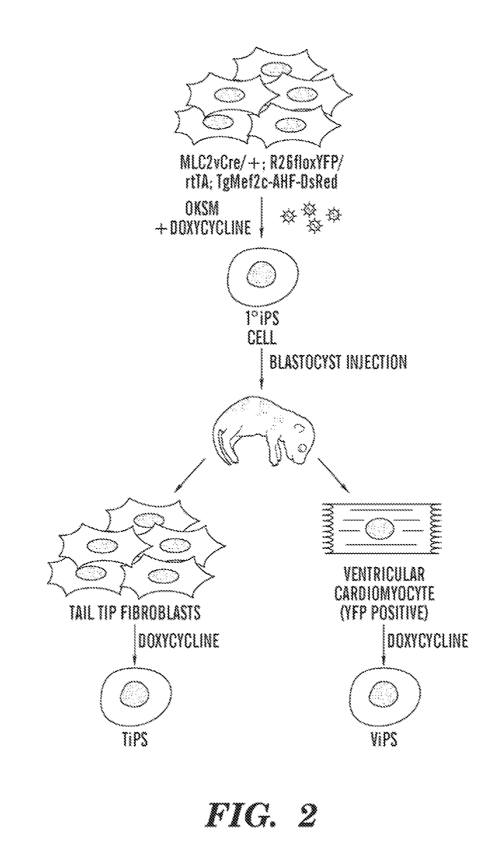

Initial attempts to generate iPS cells from ventricular myocytes by directly infecting the cells with retroviruses or lentiviruses to overexpress the reprogramming factors—Klf4, Oct4, Sox2 and Myc—were not successful, most likely due to low infection efficiency (data not shown). The inventors herein then employed an inducible secondary iPS cell system (FIG. 2). This approach also allowed the direct examination of genetically matched iPS cells from ventricular myocytes (VM) and tail tip fibroblasts (TTF) eliminating the potentially confounding effects of varying integration sites or copy numbers of the reprogramming transgenes on the differentiation potential of the iPS cells.

Generation of iPS Cells from Ventricular Cardiomyocytes and Tail Tip Fibroblasts, Respectively.

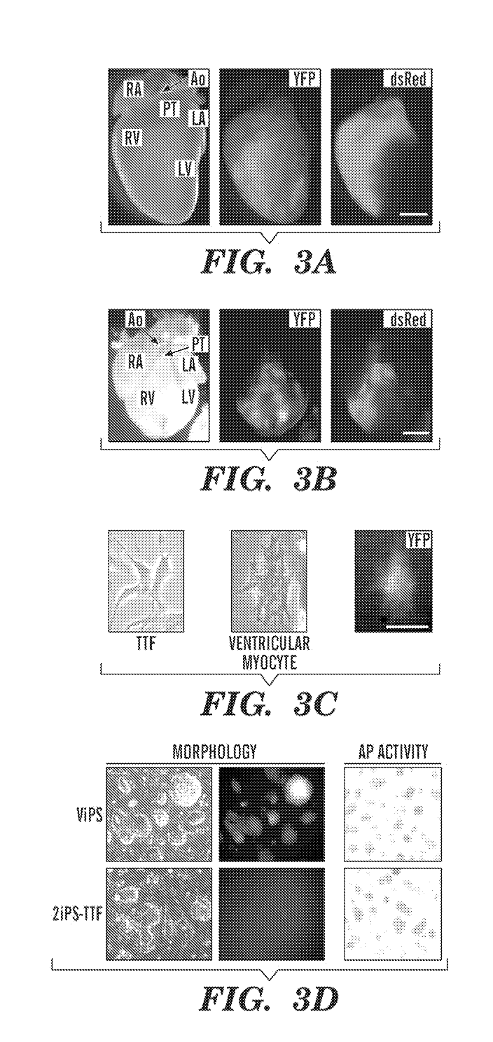

First, the inventors collected cardiac fibroblasts from neonatal pups with a compound genotype Mlc2v; Rosa26^{floxYFP/rtTA}; TgMef2c-AHF-dsRed. The Mlc2v^{Cre} knock-in allele (16) and a Cre-dependent conditional Rosa26^{floxYFP} reporter allele (with a loxP-flanked stop signal before the YFP cDNA) (17) were used to permanently label ventricular myocytes (FIG. 3A). The Rosa26^{rtTA} knock-in allele constitutively expresses rtTA, which encodes the transactivator for doxycycline-inducible promoter (18). A Mef2c-AHF-dsRed transgene in the cells labels Islet-1 cardiovascular progenitors of the anterior heart field (AHF) and their descendents (FIG. 3A) (3, 19). The inventors then generated primary iPS cell lines from cardiac fibroblasts by transducing them with lentiviruses that express the reprogramming factors Klf4, Oct3/4, Sox2, and Myc under the control of a doxycycline-inducible promoter. When injected into wild-type blastocysts, the primary iPS cells rendered chimeric postnatal pups. Analysis of the chimeras revealed that the genetic marks in the iPS cell derivatives within the heart manifested the same expression pattern as that in the original transgenic mouse model (Mlc2v^{Cre}-Rosa26^{floxYFP} in the ventricles and Mef2c-AHF-dsRed in the AHF-derived structures), indicating that the double reporter system remained active and faithful (FIG. 3B). YFP-positive ventricular myocytes as well as tail tip fibroblasts (YFP-negative) (FIG. 3C) were isolated from a single chimeric postnatal pup and plated on a mouse embryonic fibroblast (MEF) feeder cell layer under ES cell conditions with doxycycline to generate genetically matched secondary iPS cells from VM and TTF. Within two weeks, cells with an ES cell-like morphology emerged (FIG. 3D).

The inventors started with cardiac fibroblasts from neonatal pups with a genotype of Mlc2v^{Cre/+}; Rosa26^{YFP/rtTA}; TgMef2c-AHF-DsRed. As mentioned above, the Mlc2v^{Cre} knock-in allele and the conditional Rosa26^{YFP} reporter allele were used to label ventricular myocytes (FIG. 2). The Rosa26^{rrTA} knock-in allele constitutively expresses rtTA, which encodes the transactivator for doxycycline-inducible promoter (Hochedlinger, Yamada et al. 2005). A Mef2c-AHF-dsRed transgene in the cells labels the cardiac progenitor cells from the anterior heart field (AHF) and their descendents (Domian, Chiravuri et al. 2009). The inventors obtained primary iPS cells from cardiac fibroblasts by lentiviral transduction of doxycycline inducible Klf4, Oct4, Sox2 and c-Myc transgenes. Chimeras were generated from the primary iPS cells through blastocyst injection. Ventricular myocytes and tail tip fibroblasts were collected from neonatal chimeric pups and cultured in the presence of doxycycline to get the secondary iPS (2iPS) cells. There has been the integration of the reprogramming transgenes in those cells and the integration sites and copy numbers of the transgenes as well as genetic background are the same in different cell types from one chimera.

At least 30 putative secondary iPS cell lines were established from ventricular myocytes (YFP⁺) and tail tip fibroblasts (YFP⁻) of one neonatal chimera, hereafter referred to as ViPS and TiPS cells respectively. For comparison, six lines of ES cells with the same genotype were established. The iPS cells demonstrate typical ES cell morphology and display alkaline phosphatase activity (FIG. 3D).

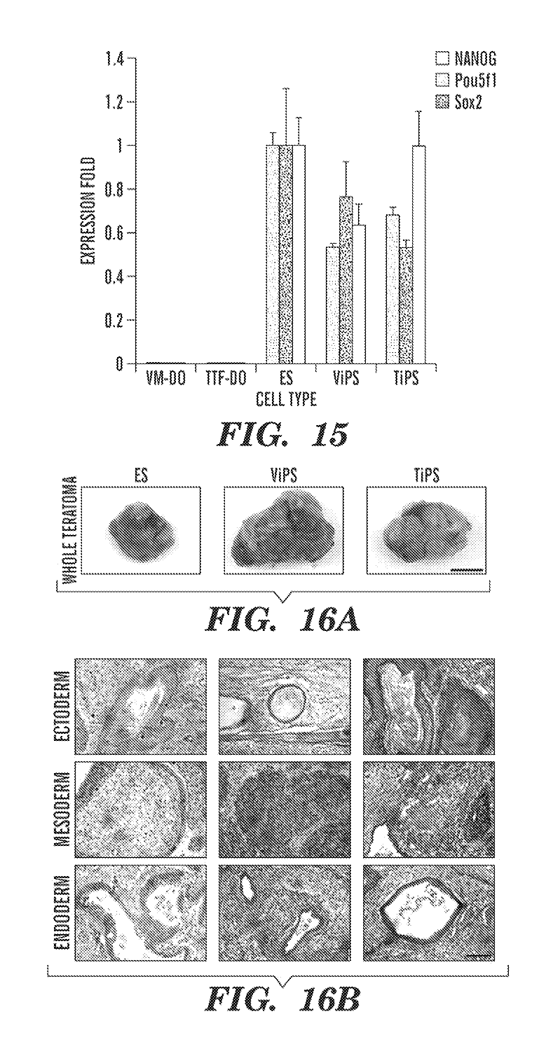

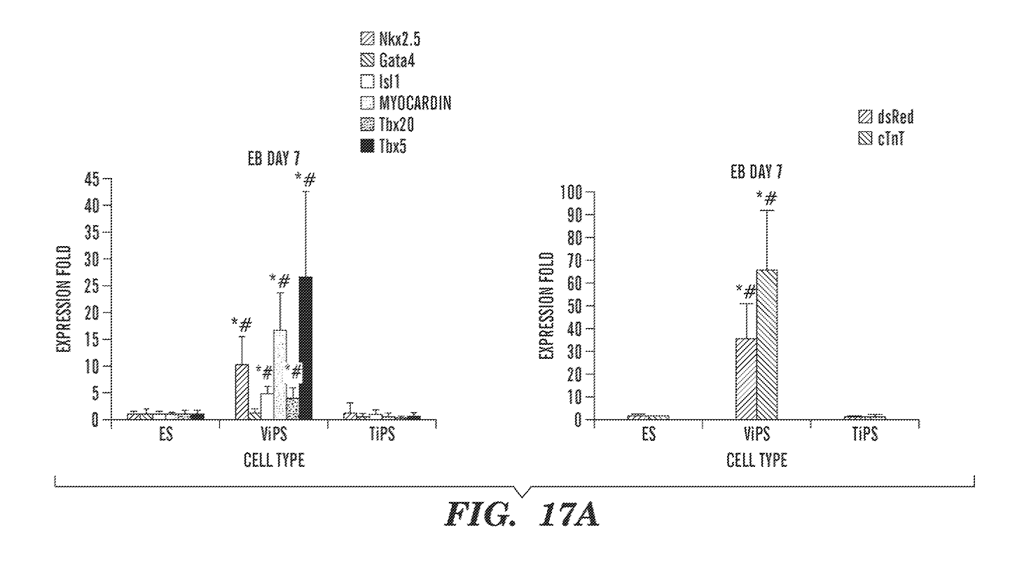

Colonies from VMs and TTFs that maintained an embryonic stem (ES) cell colony-like morphology in the absence of doxycycline were picked and expanded. Gene expression analysis demonstrated that ES cell-like colonies from both VM and TTF expressed levels of Nanog, Oct3/4 (Pou5f1) and Sox2 that were comparable to mouse ES cells (FIG. 15). These colonies displayed alkaline phosphatase activity and also demonstrated expression of SSEA1, Nanog, and Oct4 by immunofluorescence (FIG. 3D, FIG. 15). When grafted under the kidney capsules of the NOD/SCID immunodeficient mice, ES cell-like colonies from both VM and TTF differentiated into teratomas containing all three germ layers (FIGS. 16A and 16B). Therefore, by several criteria, we conclude that we had generated secondary iPS cells from ventricular myocytes, or ventricular myocyte-derived iPS cells (ViPS), and tail tip fibroblasts, or tail tip fibroblast-derived iPS cells (TiPS) (20). More than 20 lines of each were generated for analysis. For comparison, six ES cell lines were established from the starting transgenic mouse with the Mlc2v^{Cre/+}; Rosa26^{floxYFP/rtTA}; TgMef2c-AHF-dsRed genotype.

Real time polymerase chain reaction (PCR) was performed to examine the endogenous expression of the ES cell specific genes Nanog, Oct3/4 and Sox2, and the result indicated that the genes were expressed in the iPS and ES cells at comparable levels (FIG. 15 and data not shown). The presence of the ES cell surface marker protein SSEA1 and transcription factor proteins Nanog and Oct3/4 was confirmed by immunofluorescence. SSEA1 expression was detected in 2iPS-VM (ViPS) and TiPS cells (date not shown), as well as Nanog and Oct4 expression was detected in 2iPS-VM (ViPS) and TiPS cells (data not shown).

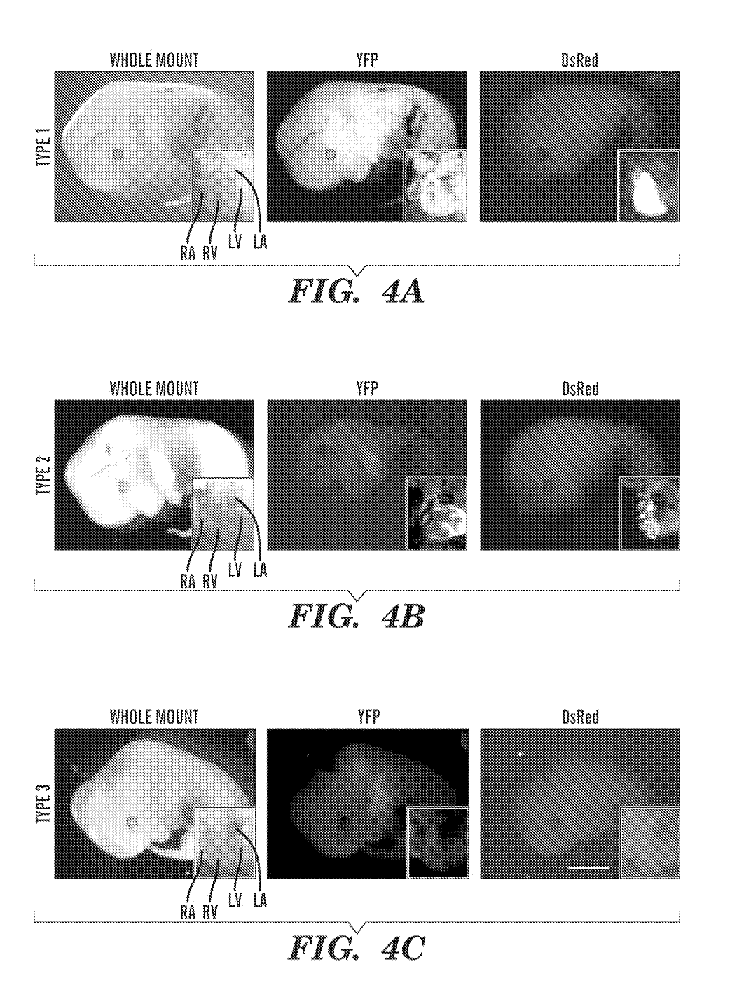

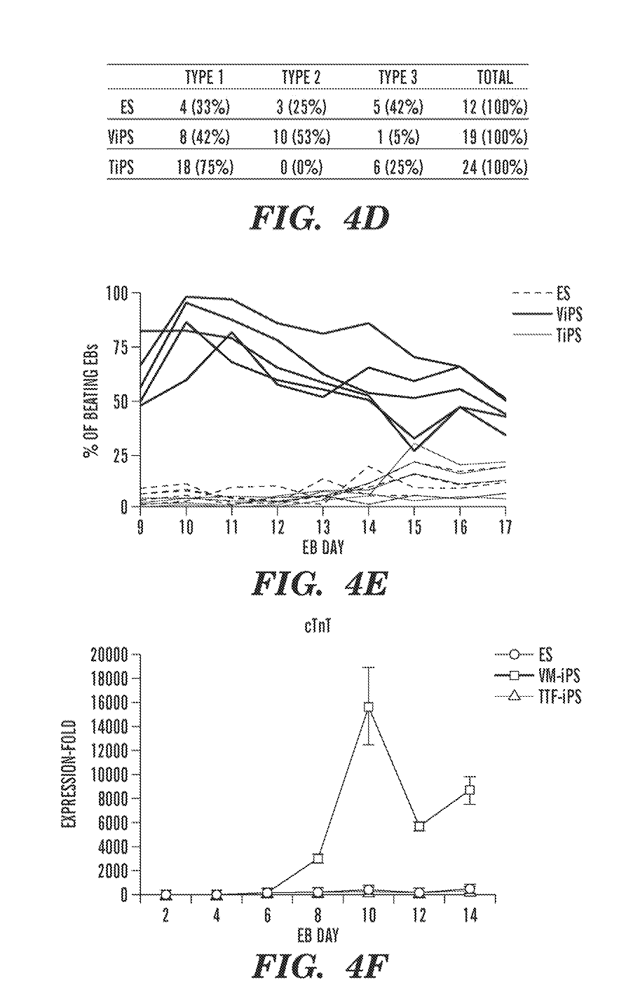

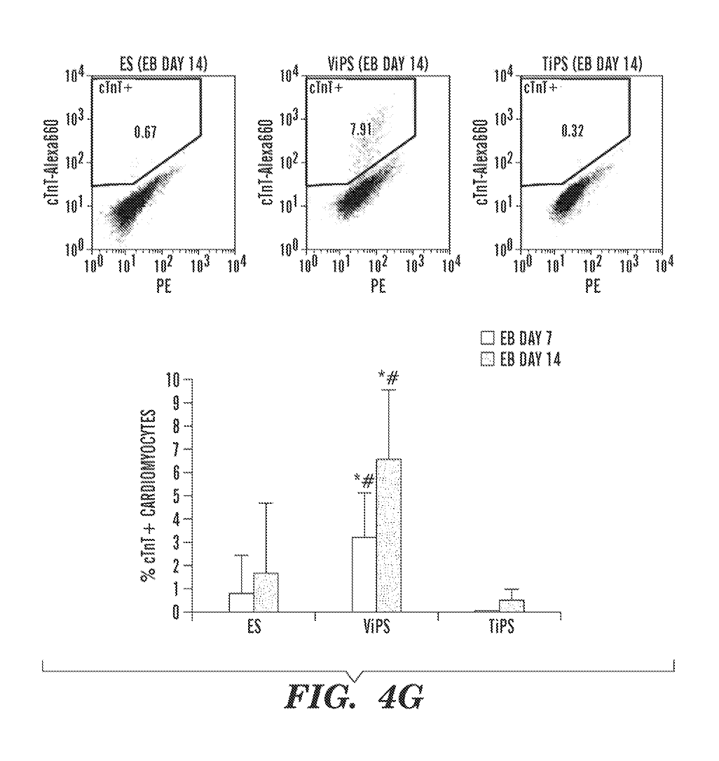

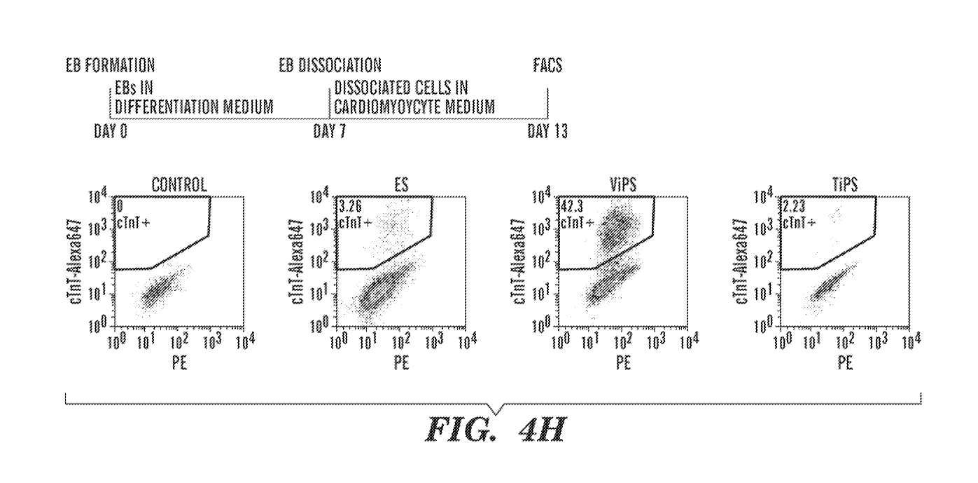

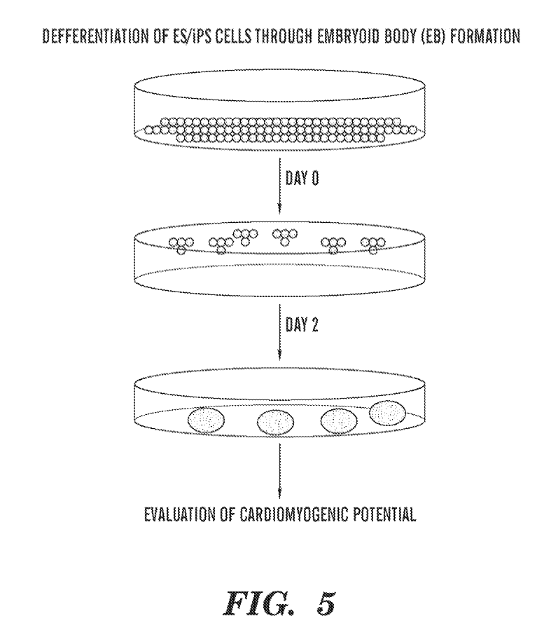

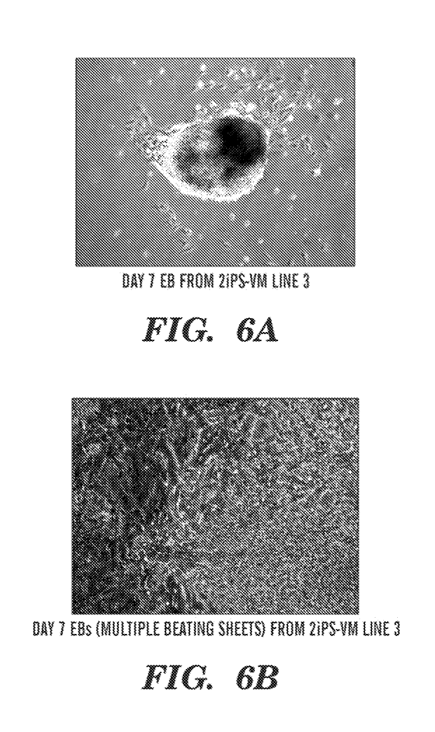

To test their pluripotency, iPS cells were injected into 3.5-day wild-type blastocysts, which were subsequently transplanted to pseudo-pregnant foster mothers. The inventors examined the developmental potential of ViPS and TiPS cells in the in vivo context by injecting them into 2n blastocysts for chimera formation (21, 22). In order to facilitate the detection of cells that had contributed to the chimera, the conditional reporter Rosa26^{floxYFP} in ES cells and TiPS cells was activated by transient expression of Cre recombinase prior to injection. Embryos were examined at embryonic day 13.5 (E13.5). Chimeric embryos at E13.5 could be obtained from all tested ViPS cell lines (n=10) and TiPS cell lines (n=6), consistent with our conclusion that we had generated bona fide secondary iPS cell lines. Interestingly, the inventors noticed that the pattern of iPS cell contribution to the chimeras could be roughly categorized into three types (FIGS. 4A, 4B): type 1 chimeras demonstrated a ≧50% contribution to the chimera overall and a similar contribution to the heart; type 2 chimeras demonstrated a <50% contribution overall but a much higher contribution to the heart; and type 3 embryos that demonstrated a <50% contribution overall with an equal or lesser contribution to the heart. In contrast to those from ES and TiPS cell lines, over half chimeras (10 out of 19) from ViPS cell lines belong to type 2 in which the contributions of the iPS cells were concentrated in the heart, suggesting a predisposition to differentiate into cardiac lineages in the context of the developing embryo (FIG. 4C).

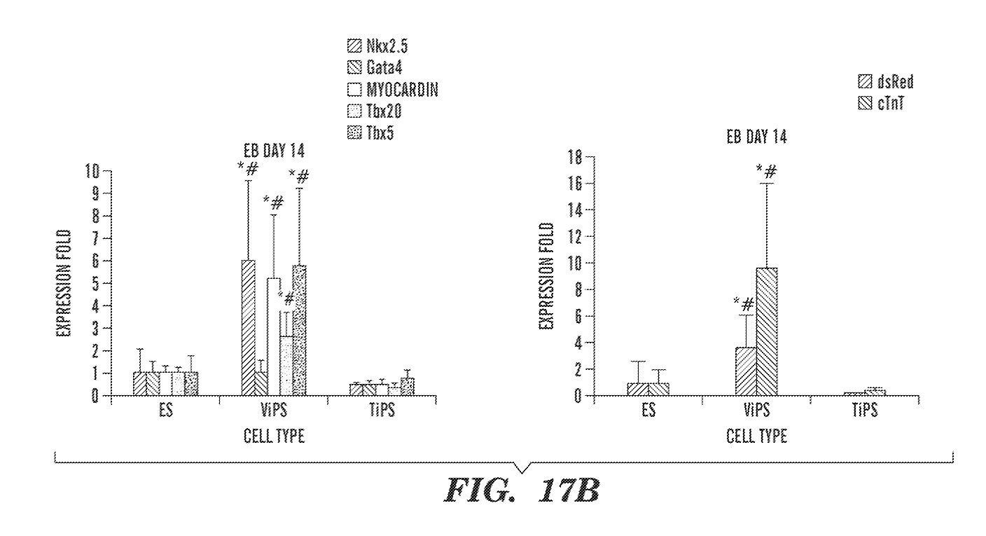

In order to assess their differentiation potential in vitro, the inventors differentiated ViPS and TiPS cell lines by embryoid body (EB) formation and compared them with mouse ES cells established from the starting transgenic mouse. ViPS cells, TiPS cells, and ES cells differentiated into EBs of comparable size (data not shown). Strikingly, while the EBs derived from ES and TiPS cells did not start contracting until day 8, about 17% of the EBs from ViPS cells started beating as early as day 6, and on day 8, about 72% EBs were contracting (data not shown). The extent of contraction also increased along the time course of differentiation (data not shown). After plating onto gelatin-coated dishes on day 7, the number of spontaneously beating EBs were monitored daily. At all examined time points, the overall percent of contracting EBs from ViPS cells were higher than ES and TiPS cells (FIG. 4E). This was consistent with the expression levels of the cardiomyocyte marker, cardiac Troponin T (cTnT) along the time course (FIG. 4F). In order to quantify the numbers of cardiomyocytes from ViPS cells, TiPS cells, and mouse ES cells, the inventors performed intracellular fluorescence activated cell sorting (FACS) using an antibody specific for cTnT (23). On day 7 and day 14, a significantly higher percent of cTnT+ cardiomyocytes were measured in EBs derived from ViPS cell lines than those derived from mouse ES or TiPS cell lines (FIG. 4G). Consistently, the expression levels of most examined cardiogenic transcription factors, including Nkx2.5, Myocardin, Tbx20 and Tbx5, as well as cTnT were significantly higher in the EBs of ViPS cell lines than ES and TiPS cell lines on both day 7 and day 14 (FIGS. 17A, 17B). To improve the cardiomyogenesis in the differentiating stem cells, we dissociated day 7 EBs and cultured the dissociated cells in a medium specialized for cardiomyocytes (FIG. 4H). The modified protocol dramatically increased the yield of cardiomyoctes from ViPS cells. When examined six days after plating the cells dissociated from day 7 EBs, contracting cells could be detected throughout the cultures of most ViPS cell lines (data not shown). Intracellular FACS analysis indicated that up to 42% of the cells were cTnT+ cardiomyocytes, which was >10-fold of the maximum reached by ES or TiPS cells (FIG. 4H).

### Example 2

### Evaluation of the Cardiomyogenic Potential of ViPS Cells in Comparison to TiPS and ES Cells

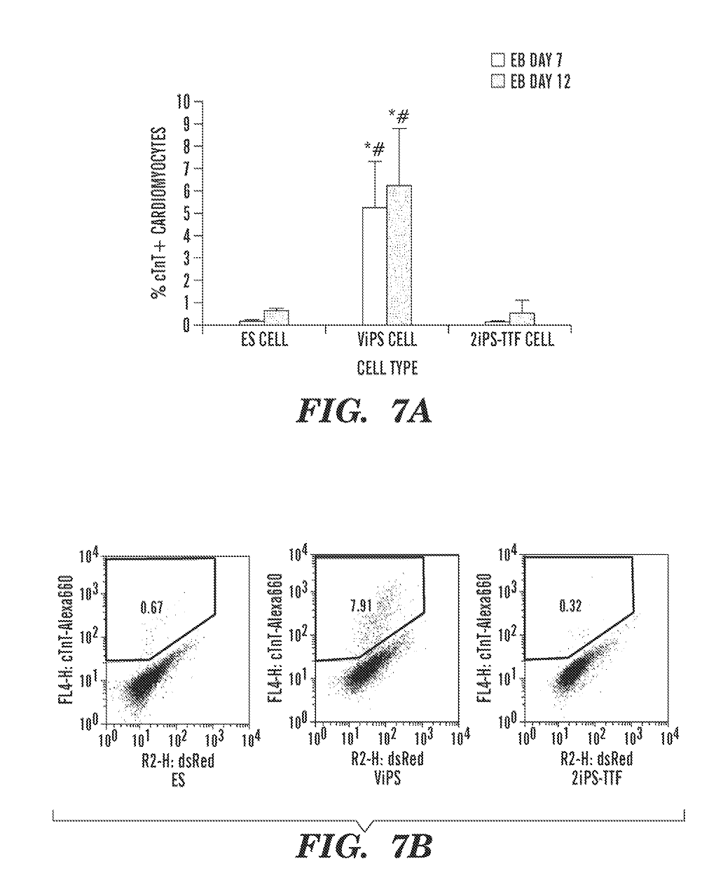

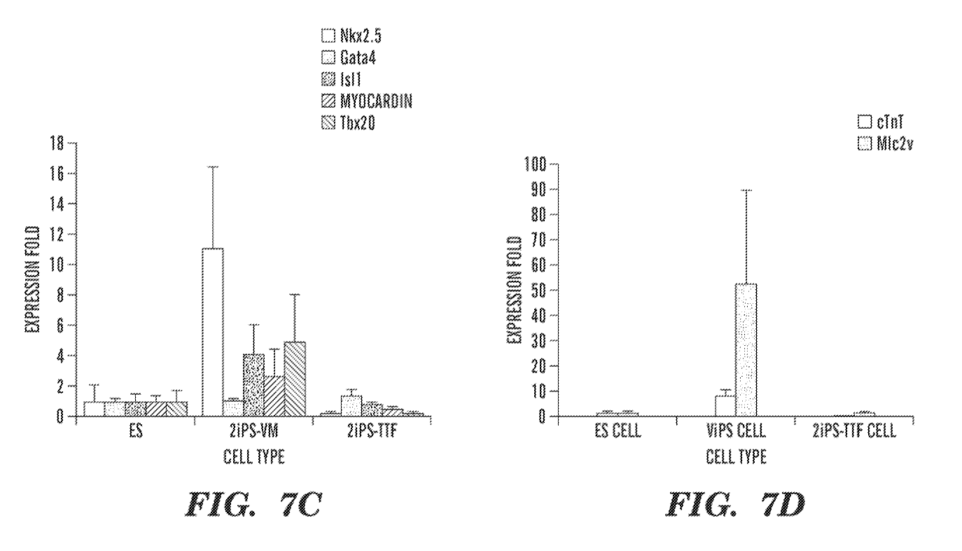

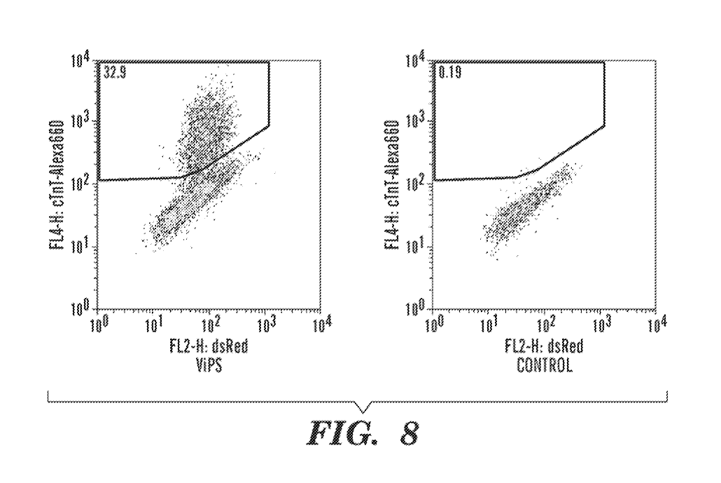

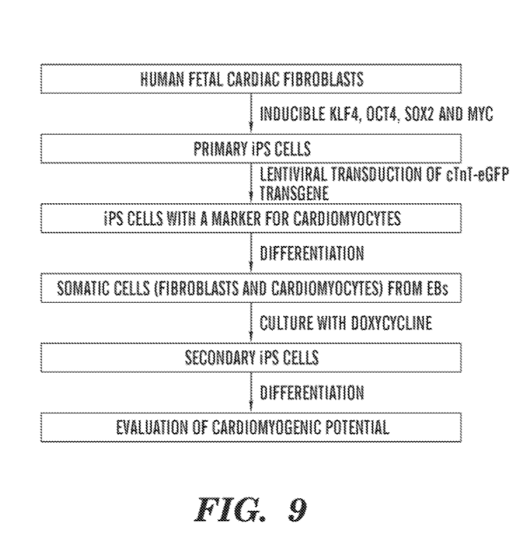

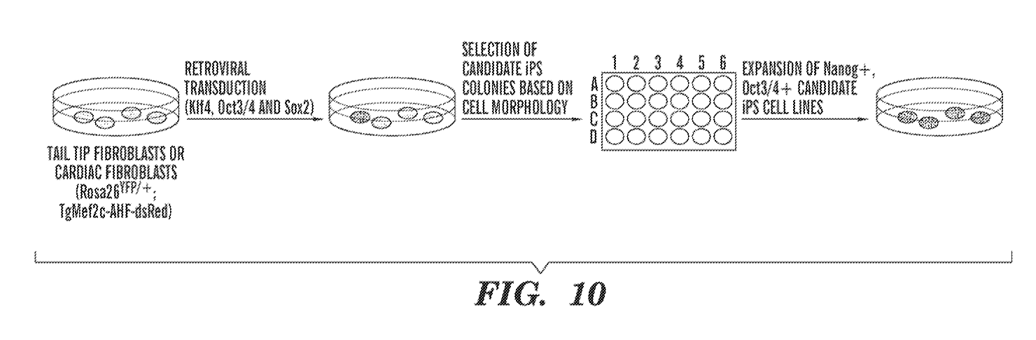

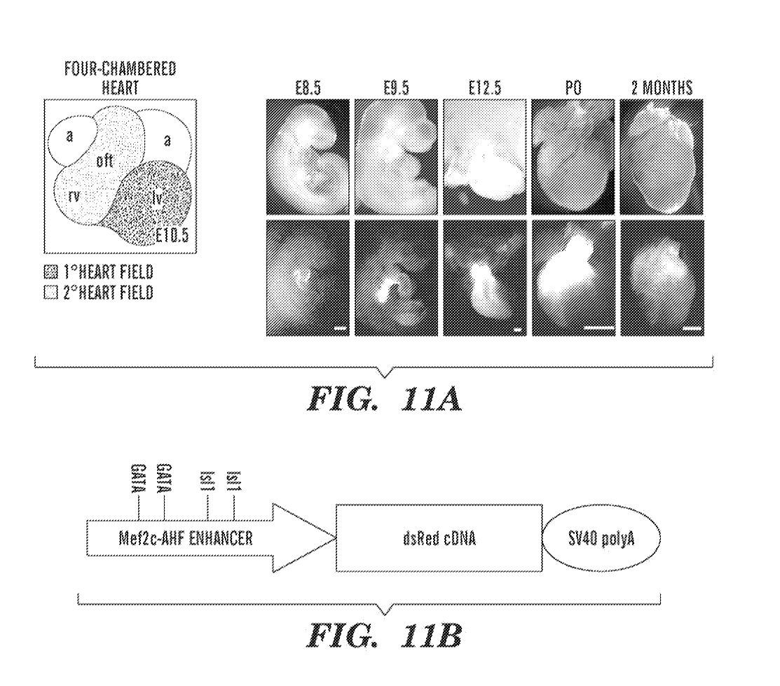

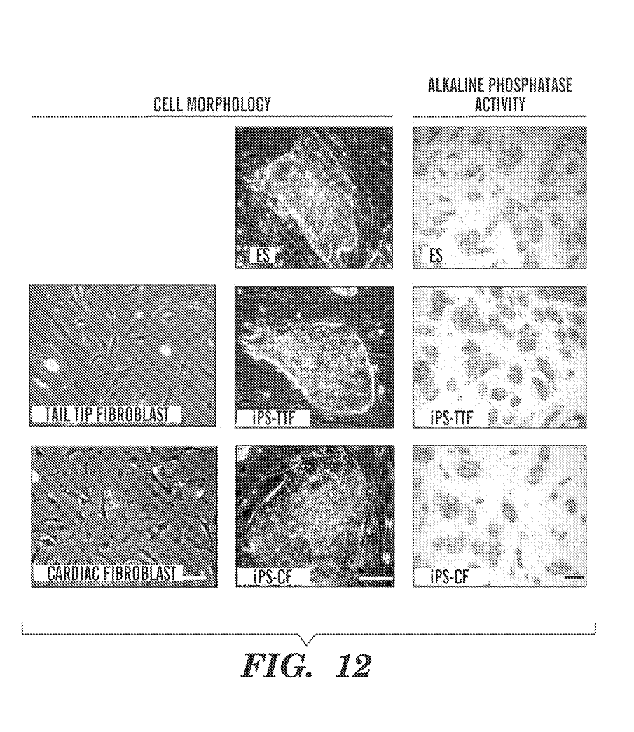

Three ViPS and TiPS cell lines, in parallel with three ES cell lines, were differentiated through embryoid body (EB) formation. All the three ViPS cell lines, and two of the three TiPS cell lines can contribute to chimeras after blastocyst injection. While the EBs from ES and TiPS cells did not start beating until day 8, about 20% of the EBs from ViPS cells were beating as early as day 6, and on day 7, about 80% EBs from two of the three ViPS cell lines were beating. To score cardiomyogenesis quantitatively, the inventors used intracellular fluorescence activated cell sorting (FACS) with the antibody against cardiac Troponin T (cTnT). On day 7, there were very few cTnT+ cardiomyocytes in the EBs from ES or TiPS cells (0.13% and 0.096%, respectively). In contrast, 5.22% of the cells in the EBs from ViPS cells were cTnT+ at this stage (FIG. 7A). On day 12, when the number of cTnT+ cardiomyocytes usually peaks in EBs, the percentage of cTnT+ cardiomyocytes from ViPS cells were over 10-fold of that from ES cells and TiPS cells (0.6% and 0.52% for ES and TiPS cells, respectively, and 6.2% for ViPS cells, FIG. 7A). Immunostaining with a cTnT antibody on the cells from the ES, ViPS and TiPS EBs confirmed the intracellular FACS analysis result (FIG. 7B). In agreement with more cardiomyocytes from ViPS cells than ES and TiPS cells, higher expression of most examined cardiogenic genes, including Nkx2.5, Islet1, Myocardin and Tbx20 was also detected in the EBs from ViPS cells (FIG. 7C). Interestingly, when the day 7 EBs from one ViPS cell line were dissociated and cultured on gelatin for another 7 days with a medium specialized for cardiomyocytes, almost 33% of the cells turned out to be cTnT+ cardiomyocytes (FIG. 8).

### Example 3

### The Majority of Cardiomyocytes from ViPS are of a Ventricular Identity

The inventors also performed real-time PCR to examine the expression of cTnT and Mlc2v, which are pan-cardiac and ventricular specific markers respectively, in the whole EBs on day 7. While the expression of cTnT in ViPS EBs was about 8-fold of that in ES cells, the expression of Mlc2v in ViPS EBs is about 52-fold (FIG. 7D). The preferentially higher expression of the ventricular specific marker over the pan-cardiac marker in ViPS cell EBs than ES cell EBs is consistent with a higher portion of ventricular myocytes among the cardiomyocytes from ViPS cells.

The inventors also demonstrated by immunocytofluorescence with antibodies against Mlc2v [ventricular marker] and Sarcolipin or ANF [atrial markers] together with that against cTnT [pan-cardiac marker]; that the ViPS cells are of a ventricular identity (data not shown).

The inventors have demonstrated that there is a higher expression of some cardiogenic genes at baseline in ViPS cells and an earlier initiation of a more robust cardiogenic program. Additionally, the inventors demonstrated that ViPS cells spontaneously differentiate into ventricular cardiomyocytes and express cardiac troponin T (cTnT) at day 12 EBs (data not shown).

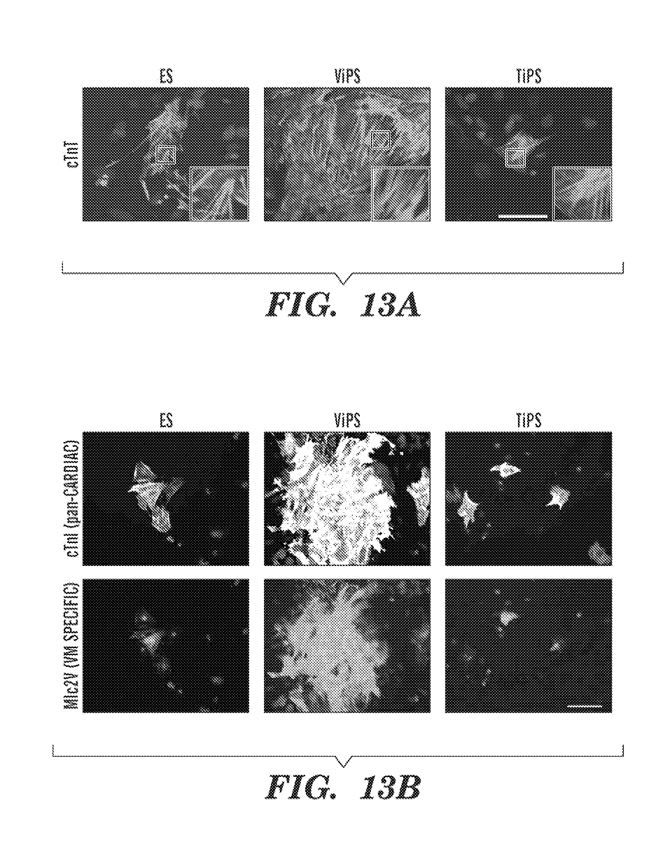

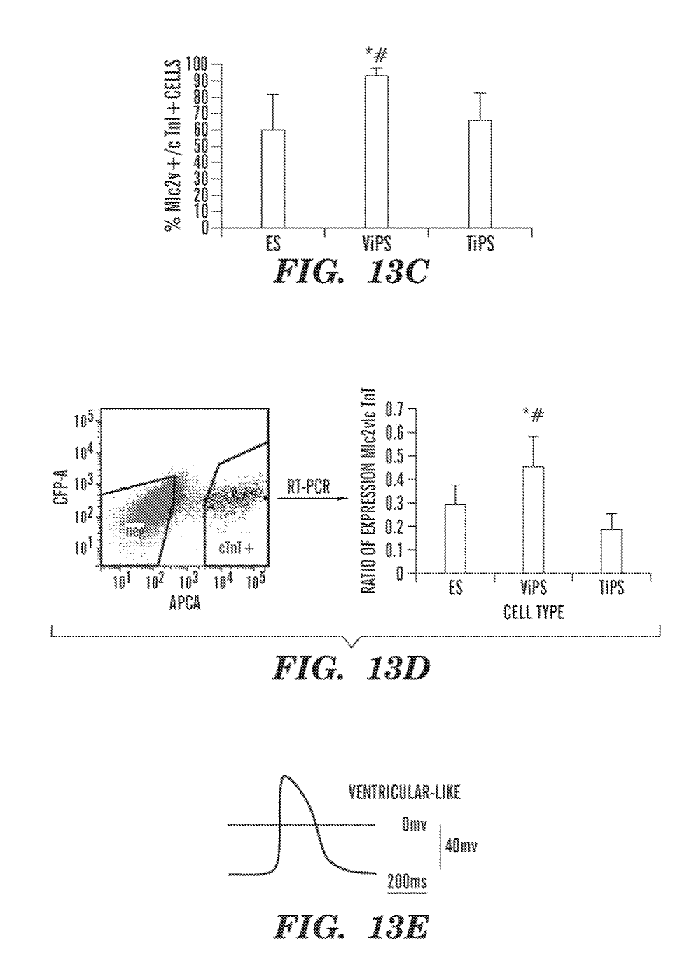

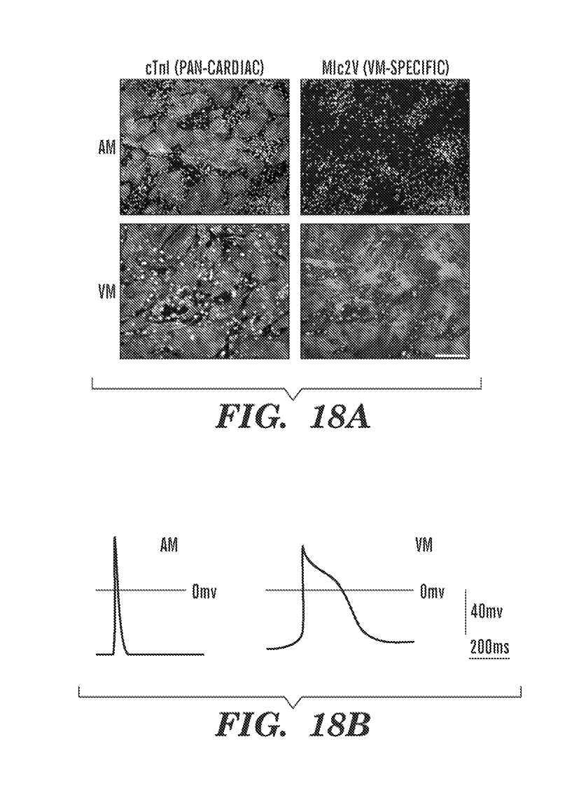

The inventors also characterized the cardiomyocytes which were derived from ViPS. Immunofluorescence assays detected cells positive for the pan-cardiomyocyte-specific marker cTnT and cardiac troponin I (cTnI) from the EBs of all ViPS cells, TiPS cells, and ES cells (FIGS. 13A, 13B), and the well-organized pattern of cTnT (FIG. 13A, insets) and cTnI (data not shown) characteristic of cardiomyocytes could be found. Consistent with the higher cardiomyogenic potential of ViPS cells, there were a greater number of cells positive for cardiac troponin T and I in the cells derived from ViPS cells than TiPS cells or ES cells (FIG. 3, A and B). To determine the type of cardiomyocytes, the inventors performed double immunostaining with a cTnI antibody that recognizes both atrial and ventricular myocytes and an Mlc2v antibody that specifically recognizes ventricular myocytes (FIG. 18A). 92% (average of six lines, with four lines >90% and the maximum 99.5%) of the cTnI⁺ cardiomyocytes derived from ViPS cells also stained with the ventricular myocyte marker Mlc2v (FIGS. 13B and 13C), while the percent of cardiomyocytes that stained with Mlc2v was significantly lower for cardiomyocytes derived from ES (59%, average of six lines) cells or TiPS cells (62%, average of six lines), suggesting that iPS cells derived from ventricular myocytes have a tendency to re-differentiate specifically into ventricular cardiomyocytes. Consistently, gene expression analysis of the FACS-purified cTnT⁺ fraction derived from ViPS cells demonstrated a higher Mlc2v/cTnT ratio of expression levels than the cTnT⁺ fraction from TiPS cells or ES cells (FIG. 13D). Indeed, when we performed patch-clamp recordings to delineate the electrophysiological properties of ViPS cell-derived cardiomyocytes, they predominantly displayed ventricular-like action potentials with a rapid upstroke, distinct plateau phase, and stable resting membrane potential (FIG. 13E and FIG. 18B) (4, 24, 25).

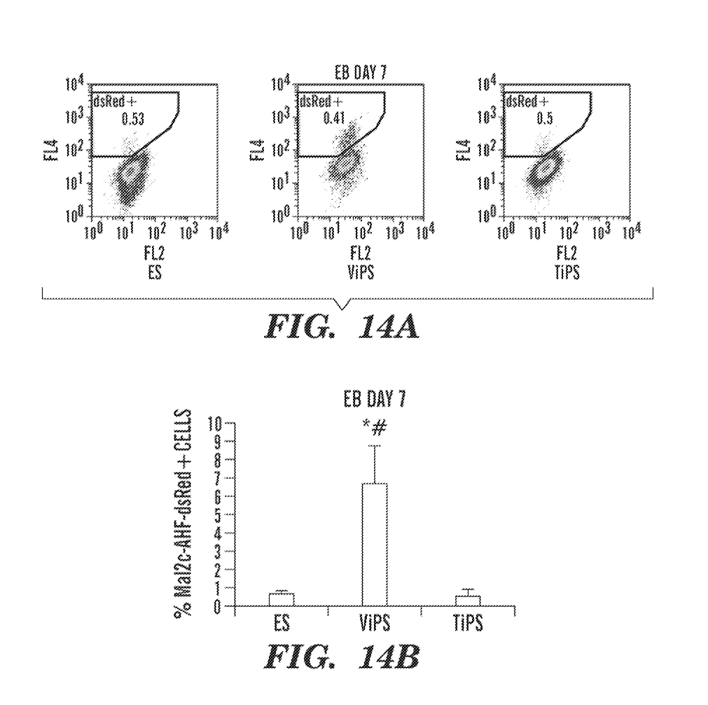

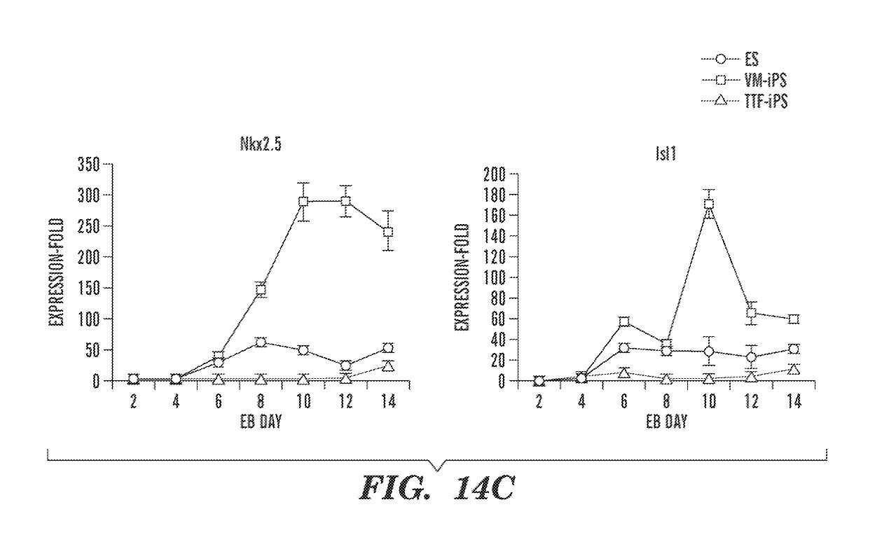

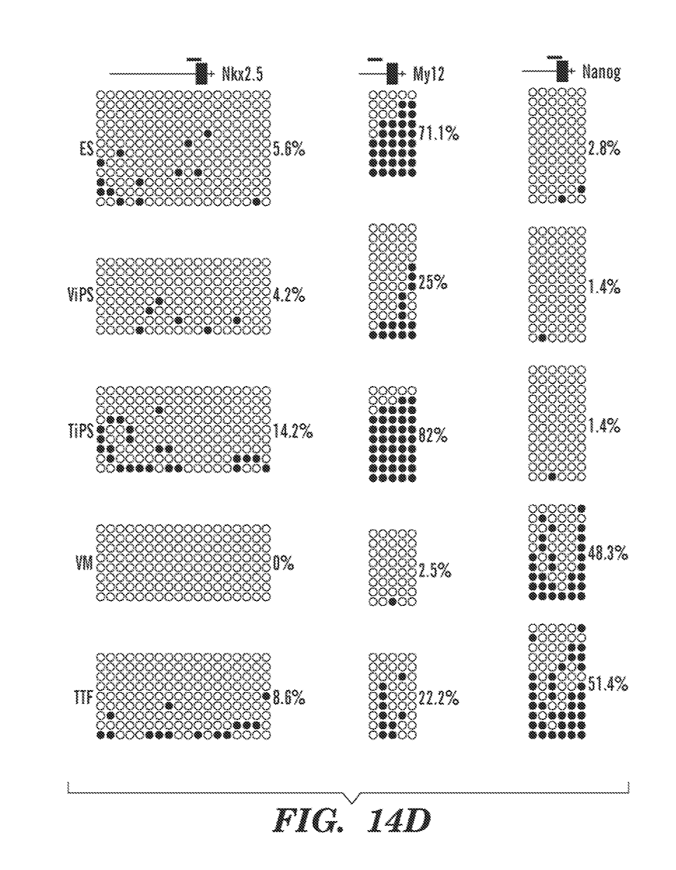

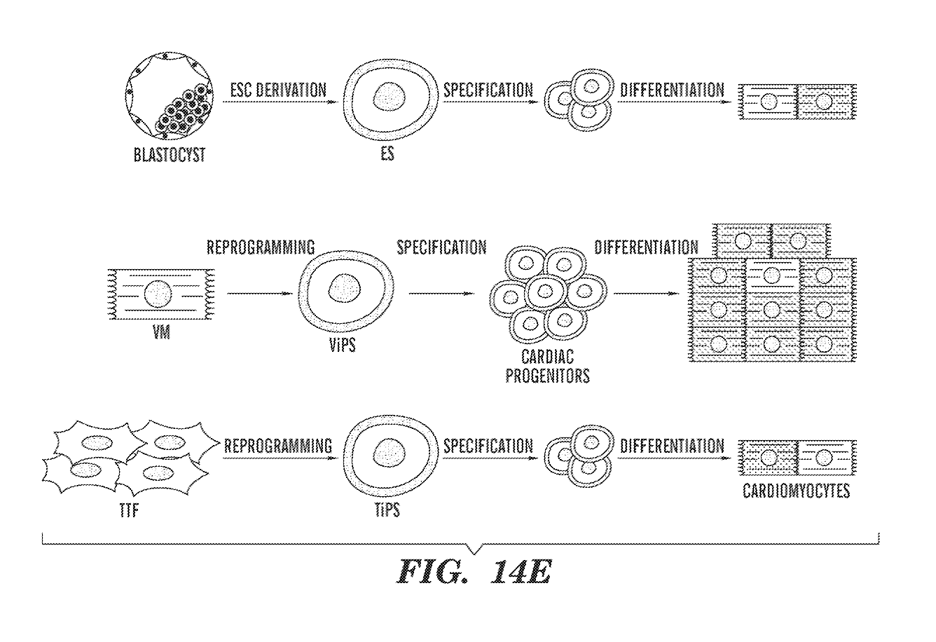

The lineage from anterior heart field progenitor to ventricular cardiomyocyte proceeds through a distinct committed ventricular progenitor (CVP) cell intermediate marked by the expression of Islet 1 and Nkx2.5 (3). To gain insight into the pathway from pluripotent stem cell to ventricular myocyte in ViPS cells, the inventors analyzed early stage EBs for Mef2c-AHF-dsRed positive cells by FACS analysis. Analysis of day 7 EBs demonstrated a higher percent (>10 fold) of dsRed⁺ cells in the EBs from ViPS cells than genetically matched TiPS cells (FIGS. 14A, 14B). Furthermore, gene expression analysis of early stage EBs revealed a higher expression of Nkx2.5 and Islet in the EBs of ViPS cells from day 6 onward (FIG. 14C) suggesting that the increased ventricular myogenesis in ViPS cells occurs via an increased number of CVPs (FIGS. 14A, 14B). Overall, these results highlight the importance of the obtaining a higher yield of committed ventricular progenitors in order to produce abundant numbers of ventricular myocytes from pluripotent stem cells.

The inventors next investigated whether the enhanced ventriculogenic potential of ViPS cell lines could be explained by differences in the DNA methylation patterns of known ventriculogenic genes (26-28). Reminiscent of their parental cells, there is considerably less DNA methylation in the promoter regions of Nkx2.5 and Myl2 (the gene encoding the ventricular type of myosin light chain) in ViPS cells than ES cells and TiPS cells, although the promoter of Nanog has similar DNA methylation in the different cell types (FIG. 14D). These results are consistent with the reports that iPS cells retain epigenetic memory of the starting cell type and suggest that the increased ventriculogenic potential of ViPS cells is mediated in part by distinct epigenetic signatures leftover from the parental ventricular myocyte.

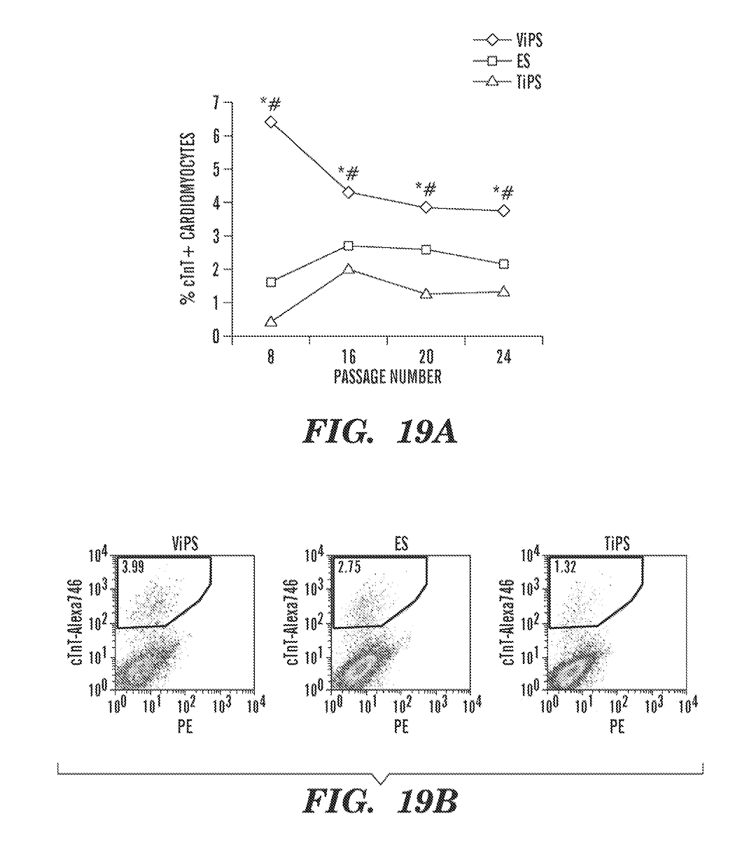

The inventors investigated what the effects of continuous passaging of ViPS cells would have on their “cardiomyocyte memory”. The molecular and functional differences in B-cell-, T-cell-, and granulocyte-derived iPS cells were found to be entirely abrogated by passage number 16 (13, 14). The inventors subjected ViPS cells, TiPS cells, and ES cells to additional rounds of passaging under identical culture conditions and assessed the stability of the enhanced cardiomyogenic potential of ViPS cells with repeated passage. At passage 16, the percent of cTnT⁺ cells from ViPS cells declined while the percent of cTnT⁺ from TiPS cells increased slightly to reduce the magnitude of difference in differentiation potential (FIGS. 19A, 19B); the percent of cTnT⁺ cells from ES cells remained relatively constant. At passage 24, however, ViPS cells retained a discernibly stronger cardiomyogenic potential that TiPS cells suggesting that, for some somatic cell types, “stem cell memory” can be stable out to higher passage numbers.

The inventors discoveries as disclosed herein is the first report of the direct reprogramming of cardiomyocytes into iPS cells. The use of an irreversible genetic labelling system (Mlc2v^{Cre}-Rosa26^{floxYFP}) allowed us to definitively identify the starting subtype of cardiomyocytes, the ventricular myocyte. The inventors also find that stem cell memory for ventricular cardiomyocytes can be stable and specific to a particular subtype of cardiomyocyte. Highly ventriculogenic pluripotent stem cells represent a remarkably efficient and renewable source of abundant numbers of CVPs and a relatively homogenous population of ventricular myocytes; whereas ES cells and iPS cells derived from fibroblasts tend to produce a heterogeneous mixture of cardiomyocytes and relatively low yields. The inventors discoveries findings provide the first illustration that epigenetic memory in iPS cells may be advantageously exploited to generate a desired somatic cell type and suggest that the derivation of iPS cells from ventricular myocytes may be a robust method by which abundant numbers of chamber-specific myocytes can be derived for disease-modelling, drug discovery and future applications in cell-based therapeutics.

### Example 4

The inventors have demonstrated herein that iPS cells produced by reprogramming somatic cells from the heart have a higher cardiomyogenic potential as compared to iPS cells produced by reprogramming somatic cells that are from other sources, e.g., non-heart somatic cells.

### REFERENCES

All references cited herein and throughout the specification are incorporated in their entirety by reference.
Aoi, T., K. Yae, et al. (2008). “Generation of pluripotent stem cells from adult mouse liver and stomach cells.” Science 321(5889): 699-702.
Chen, J., S. W. Kubalak, et al. (1998). “Selective requirement of myosin light chain 2v in embryonic heart function.” J Biol Chem 273(2): 1252-6.
Domian, I. J., M. Chiravuri, et al. (2009). “Generation of functional ventricular heart muscle from mouse ventricular progenitor cells.” Science 326(5951): 426-9.
Hochedlinger, K., Y. Yamada, et al. (2005). “Ectopic expression of Oct-4 blocks progenitor-cell differentiation and causes dysplasia in epithelial tissues.” Cell 121(3): 465-77.
Laugwitz, K. L., A. Moretti, et al. (2005). “Postnatal isl1+ cardioblasts enter fully differentiated cardiomyocyte lineages.” Nature 433(7026): 647-53.
Maherali, N., Sridharan, R, Xie, W, Utikal, J, Eminli, S, Arnold, K, Stadtfeld, M, Yachechko, R, Tchieu, J, Yaenisch, R, Plath, K and Hochedlinger, K (2007). “Directly Reprogrammed Fibroblasts Show Global Epigenetic Remodeling and Widespread Tissue contribution.” Cell Stem Cell 1(1): 55-70.
Srinivas, S., T. Watanabe, et al. (2001). “Cre reporter strains produced by targeted insertion of EYFP and ECFP into the ROSA26 locus.” BMC Dev Biol 1: 4.
Stadtfeld, M., N. Maherali, et al. (2008). “Defining molecular cornerstones during fibroblast to iPS cell reprogramming in mouse.” Cell Stem Cell 2(3): 230-40.
Takahashi, K. and S. Yamanaka (2006). “Induction of pluripotent stem cells from mouse embryonic and adult fibroblast cultures by defined factors.” Cell 126(4): 663-76.
Wernig, M., A. Meissner, et al. (2007). “In vitro reprogramming of fibroblasts into a pluripotent ES-cell-like state.” Nature 448(7151): 318-24.
1. J. A. Epstein, N Engl J Med 363, 1638 (Oct. 21, 2010).
2. M. A. Laflamme, C. E. Murry, Nat Biotechnol 23, 845 (July, 2005).
3. I. J. Domian et al., Science 326, 426 (Oct. 16, 2009).
4. A. C. Fijnvandraat, R. H. Lekanne Deprez, A. F. Moorman, Cardiovasc Res 58, 303 (May 1, 2003).
5. W. Roell et al., Nature 450, 819 (Dec. 6, 2007).
6. E. M. Hansson, M. E. Lindsay, K. R. Chien, Cell Stem Cell 5, 364 (Oct. 2, 2009).
7. T. Aoi et al., Science 321, 699 (Aug. 1, 2008).
8. S. Yamanaka, Cell 137, 13 (Apr. 3, 2009).
9. C. Mauritz et al., Circulation 118, 507 (Jul. 29, 2008).
10. G. Narazaki et al., Circulation 118, 498 (Jul. 29, 2008).
11. J. Q. He, Y. Ma, Y. Lee, J. A. Thomson, T. J. Kamp, Circ Res 93, 32 (Jul. 11, 2003).
12. J. C. Moore et al., Biochem Biophys Res Commun 372, 553 (Aug. 8, 2008).
13. K. Kim et al., Nature advance online publication (2010).
14. J. M. Polo et al., Nat Biotechnol (Jul. 19, 2010).
15. K. Musunuru, I. J. Domian, K. R. Chien, Annu Rev Cell Dev Biol 26, 667 (Nov. 10, 2010).
16. J. Chen et al., J Biol Chem 273, 1252 (Jan. 9, 1998).
17. S. Srinivas et al., BMC Dev Biol 1, 4 (2001).
18. K. Hochedlinger, Y. Yamada, C. Beard, R. Jaenisch, Cell 121, 465 (May 6, 2005).
19. E. Dodou, M. P. Verzi, J. P. Anderson, S. M. Xu, B. L. Black, Development 131, 3931 (August, 2004).
20. N. Maherali, K. Hochedlinger, Cell Stem Cell 3, 595 (Dec. 4, 2008).
21. N. Maherali et al., Cell Stem Cell 1, 55 (Jun. 7, 2007).
22. M. Wernig et al., Nature 448, 318 (Jul. 19, 2007).
23. L. Yang et al., Nature 453, 524 (May 22, 2008).
24. J. D. Fu et al., Stem Cells Dev 19, 773 (June, 2010).
25. C. Mummery et al., Circulation 107, 2733 (Jun. 3, 2003).
26. M. Ieda et al., Cell 142, 375 (Aug. 6, 2010).
27. A. Meissner et al., Nature 454, 766 (Aug. 7, 2008).
28. T. S. Mikkelsen et al., Nature 454, 49 (Jul. 3, 2008).

1. A method of generating an induced pluripotent stem (iPS) cell which can differentiate into a ventricular cardiomyocyte comprising:
i. reprogramming a first somatic cell by inducing the expression of at least one reprogramming factor to produce a first induced pluripotent stem cell;
ii. differentiating the first induced pluripotent stem cell along a cardiogenic lineage to generate a second cardiac cell;
iii. reprogramming the second cardiac cell by inducing the expression of at least one reprogramming factor to produce a second induced pluripotent stem cell,
wherein the second induced pluripotent stem cell can differentiate into a ventricular cardiomyocyte.

2. The method of claim 1, wherein the first somatic cell is selected from a fibroblast, a skin fibroblast or cardiac fibroblast, or a cardiomyocyte.

3. The method of claim 1, wherein the first somatic cell is a human somatic cell.

4. The method of claim 1, wherein the second cardiac cell is a cardiomyocyte.

5. The method of claim 4, wherein the cardiomyocyte is a ventricular cardiomyocyte.

6. The method of claim 1, wherein inducing the expression of reprogramming factor in the first cardiac cell comprises introducing genes encoding at least one reprogramming factor operatively linked to an inducible promoter.

7. The method of claim 1, wherein inducing the expression of reprogramming factor in the second cardiac cell comprises contacting the second cardiac cell with an agent to induce expression of the genes encoding at least one reprogramming factor operatively linked to an inducible promoter.

8. The method of claim 1, wherein the reprogramming factor comprises one or more gene products selected from the group consisting of: Oct3/4, Klf4, Sox2, c-Myc, Nanog, Lin28.

9. The method of claim 1, wherein the reprogramming factor further comprises one or more gene products of a Sal4 gene.

10. The method of claim 1, wherein differentiating the first induced pluripotent stem cell along a cardiogenic lineage comprises contacting the first induced pluripotent stem cell with a cardiomyocyte differentiating agent, or agent which induces the expression of at least one gene selected from the group consisting of: cardiotrophic agents, creatine, carnitine, taurine, activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), VEGF and TGFα.

11. The method of claim 1, wherein the second cardiac cell expresses at least one of any of the following cardiac markers; Mlc2v, cTnT.

12. The method of claim 1, wherein the second iPS cell expresses at least one of any of the following markers at a significantly higher level than ES cells or a primary iPS cell; Nkx2.5, Gata4, Isl1, myocardin and Tbx20.

13. The method of claim 1, wherein the first somatic cell which is reprogrammed to a first iPS cell or the second cardiac cell which is reprogrammed to a second iPS cell is a postnatal somatic cell, or an adult somatic cell.

14. The method of claim 1, wherein the second cardiac cell which is reprogrammed to a second iPS cell is not an embryonic cell.

15. A ventricular cardiomyocyte obtained by differentiating a secondary induced pluripotent stem (iPS) cell by the method of claim 1.

16. The ventricular cardiomyocyte of claim 15, wherein the ventricular cardiomyocyte is a mammalian ventricular cardiomyocyte.

17. The ventricular cardiomyocyte of claim 15, wherein the ventricular cardiomyocyte is a human ventricular cardiomyocyte.

18. A method of treating a subject with a cardiovascular disease or disorder comprising administering to the subject a population of secondary induced pluripotent (iPS) cells or their differentiated progeny.

19. A method for assessing the toxicity of an agent on a population of ventricular cardiomyocytes, the method comprising contacting a population of secondary induced pluripotent (iPS) cells derived from cardiac cells, or their differentiated progeny with an agent, and measuring the change on the contractile effect of the agent on the population of second induced pluripotent (iPS) cells derived from cardiac cells, or their differentiated progeny in the presence of the agent.

20. The method of claim 19, wherein the differentiated progeny is a population of ventricular cardiomyocyte.

21. The method of claim 19, wherein a change in the contractile activity is an increase or decrease in at lease one contractile activity, and wherein a contractile activity is selected from the group consisting of: contractile force, contractile frequency, contractile duration and contractile stamina.
